SEQUENCE LISTING (5 sequences)

SEQ ID NO: 1 - DNA, 142090 bp, Artificial Sequence
  Description of Artificial Sequence Synthetic polynucleotide
gaggagcggctagaccccggaaacgggccccccccaaaacacaccccccgggggcgcgcgcggccctttaaaggcgggcggcgggcagcccgggccccccgcggccgcgactagcgagttagacaggcaagcactactcgcctctgcacgcacatgcttgcctgtcaaactctaccaccccggcacgctctctgtctccatggcccgccgccgccatcgcggcccccgccgcccccggccgcccgggcccacgggcgcggtcccaaccgcacagtcccaggtaacctccacgcccaactcggaacccgtggtcaggagcgcgcccgcggccgccccgccgccgccccccgccccccattagcatgcccctcccgccgacgcaacaggggcttggcctgcgtcggtgccccggggcttcccgccttcccgaagaaactcattaccatacccggaaccccaggggaccaatgcgggttcattgagcgacccgcgggccaatgcgcgaggggccgtgtgttccgccaaaaaagcaattaacataacccggaaccccaggggagtggttacgcgcggcgcgggaggcggggaataccggggttgcccattaagggccgcgggaattgccggaagcgggaagggcggccggggccgcccattaatgagtttctaattaccatccgggaagcggaacaaggcctctgcaattttttaattcccagcccgggaagggggcggcccggcccactgggcgggggttaccgcccagtgggccgggccccgacgactcggcggacgctggttggccgggccccgccgcgctggcggccgccgattggccagtcccgccctccgagggcgggcccgcctcgggggcgggccggctccaagcgtatatatgcgcggctcctgccatcgtctctccggagagcggcttggtgcggagctcccgggagctccgcggaagacccaggccgcctcgggtgtaacgttagaccgagttcgccgggccggctccgcgggccagggcccgggcacgggcctcgggccccaggcacggcccgatgaccgcctcggcctccgccacccggcgccggaaccgagcccggtcggcccgctcgcgggcccacgagccgcggcgcgccaggcgggcggccgaggcccagaccaccaggtggcgcacccggacgtggggcgagaagcgcacccgcgtgggggtcgcgggggtcgcgggggtcgcggggggcttcggcgccccctccccgcccgcgcgtcgcaggcgcaggcgcgccaggtgctctgcggtgacgcgcaggcggagggcgaggcgcggcggaaggcggaaggggggagggggggtgggaggggttagccccgccccccgggcccgcgccgggcggtggggaccgggggcggggggcggcggcggtgggccgggcctctggcgccggctcgggcggggggctgtccggccagtcgtcgtcgtcgtcgtcggacgcggactcgggaacgtggagccactggcgcagcagcagcgaacaagaaggcgggggcccctggcggggggcggcggcggggcggccgcgggcgcgctcctgaccacgggttccgagttgggcgtggaggttacctgggactggcggttgggaccgcgcccgtgggcccgggcggccgggggcggcgggggccgcgatggcggcggcgggccatggagacagagagcgtgccggggtggtagagtttgacaggcaagcatgtgcgtgcagaggcgagtagtgcttgcctgtctaactcgctagtctcggccggggggggcccgggctgcccgccgcccgcctttaaagggccgcgcgccccccgccagtgggcccccgccttcttgttcgctgctgctgcgccagtggctccacgttcccgagtccgcgtccgacgacgacgacgacgactggccggacagccccccgcccgagccggcgccagaggcccggcccaccgccgccgccccccgcccccggtccccaccgcccggcgcgggcccggggggcggggctaacccctcccaccccccctccgccccttccgccttccgccgcgcctcgccctccgcctgcgcgtcaccgccgagcacctggcgcgcctgcgcctgcgacgcgcgggcgggggggggcgccgaagcccccccgacccccgcgacccccgcgacccccacgcgggtgcgcttctcgccccacgtccgggtgcgccacctggtggtctgggcctcggccgcccgcctggcgcgccgcggctcgtgggcccgcgagcgggccgaccgggctcggttccggcgccgggtggcggaggccgaggcggtcatcgggccggcctggggcccgaggcccgtgcccgggcccggcccgcggagccggcccggcgaactcggtctaacgttacacccgaggcggcctgggtcttccgcggagctcccgggagctccgcaccaagccgctctccggagagacgatggcaggagccgcgcatatatacgcttggagccggcccgcccccgaggcgggcccgccctcggagggcgggactggccaatcggcggccgccagcgcggcggggcccggccaaccagcgtccgccgagtcgtcggggcccggcccactgggcggtaactcccgcccagtgggccgggccgcccacttcccggtatggtaattaaaaacttgcagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgttaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttggtcccccaaatcgggggccgggccgtgcatgctaatgatattctttgggggcgccgggttggtccccggggacggggccgctccgcggtgggcctgcctcccctgggacgcgcggccattgggggaatcgtcactgccgcccctttggggaggggaaaggcgtggggtataagttagccctggcccgacggtctggtcgcatttgcacctcggcactcggagcgagacgcagcagccaggcagactcgggccgccccctctccgcatcaccacagaagccccgcctacgttgcgacccccagggaccctccgtcagcgaccctccagccgcatacgaccccccggggatcctctagggcctctgagctattccagaagtagtgaagaggcttttttggaggcctaggcttttgcaaaaagctccggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaacgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattgatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgtgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgacccgggacgagggaaaacaataagggacgcccccgtgtttgtggggaggggggggtcgggcgctgggtggtctctggccgcgcccactacaccagccaatccgtgtcggggaggtggaaagtgaaagacacgggcaccacacaccagcgggtcttttgtgttggccctaataaaaaaaactcaggggatttttgctgtctgttgggaaataaaggtttacttttgtatcttttccctgtctgtgttggatgtatcgcgggggtgcgtgggagtgggggtgcgtgggagtgggggtgcgtgggagtgggggtgcgtgggagtgggggcccacgcacccccactcccacgcacccccacacccacgcacccccgcgatacatccaacacagacagggaaaagatacaaaagtaaacctttatttcccaacagacagcaaaaatcccctgagttttttttattagggccaacacaaaagacccgctggtgtgtggtgcccgtgtctttcactttccacctccccgacacggattggctggtgtagtgggcgcggccagagaccacccagcgcccgacccccccctccccacaaacggggggcccggagagccgcggcacccggacgcgcccggaaagtctttcgcaccaccggcgatcggcacggccgcgcccccgcttttataaaggctcagatgacgcagcaaaaacaggccacagcaccacatgggtaggtgatgtaattttattttcctcgtctgcggcctaatggatttccgggcgcggtgcccctgtctgcagagcacttaacggattgatatctcgcgggcacgcgcgcccttaatggaccggcgcggggcggggggccggatacccacacgggcgggggggggtgtcgcgggccgtctgctggcccgcggccacataaacaatgactcggggcctttctgcctctgccgcttgtgtgtgcgcgcgccggctctgcggtgtcggcggcggcggcggcggtggccgccgtgttcggtctcggtagccggccggcgggtggactcgcggggggccggagggggggaggcagggggggggagggtggggatcaggacttccacttcccgtccttccatcccccgttcccctcggttgttcctcgcctcccccaacaccccgccgctttccgttggggttgttattgttgtcgggatcgtgcgggccgggggtcgccggggcaggggcgggggcgtgggcgggggtgctcgtcgatcgaccgggctcagtgggggcgtggggtgggtgggagaaggcgaggagactggggtgggggtgtcggtgggtggttgttttttcccccctgccttccaccctccggccccccgcgagtccacccgccggccggctaccgagaccgaacacggcggccaccgccgccgccgccgccgacaccgcagagccggcgcgcgcacacacaagcggcagaggcagaaaggccccgagtcattgtttatgtggccgcgggccagcagacggcccgcgacacccccccccccccgtgtggggatccggccccccgcccccgccgcccattaagggcgcgcgtgcccgcggatatcatccgttaagtgctctgcagacaggggcaccgcgcccggaaatccattaggccgcagacgaggaaaataaaattacatccctacccatgtggtgctgtggcctgtttttgctgcgtcatctgagcctttataaaagcgggggcgcggccgtgccgatcgccggtggtgcgaaagactttccgggcgcgtccgggccccccgccgctaaaccccatcccgcccccgggaccccacatataagcccccagccacacgcaagaacagacacgcagaacggctgtgtttattttaaataaaccgatgtcggaataaacaaacacaaacacccgcgacggggggacggcggggacggagggaggggggggacgggggacggaaacagacacaaaaaacaaccacaaaaaaaaaaaacaaccacccaccgacaccccccccccagtctcctcgccttctccccccaccccacgcccccactgagcccggtcgatcgacgagcacccccgcccccgcccccgcccctgccccggcgacccccggcccgcacgatcccgacaacaataatccgtcccccgtccccccctccctccgtcccctccgtcccccctcgcgggggtttgtgtttgtttattccgacatcggtttatttaaaataaacacagccgttctgcgtgtctgttcttgcgtgtggctgggggcttatatgtggggtcccgggggcgggatggggtttagcggcggggggcggcgcgccggacggggcgctggagatagcggcccccggggaccgggggaccggggctgggtatcccgaggtggggatgtgggcgggggtgcgcgggaggggtcggtggtgggggtggtggtggtgggggtagtaggaatggtggggggggggagggcgctggttggtcaaaaaagggagggacgggggccggcagaccgacggcgacaacgctccccggcggccgggtcgcggctcttacgagcggcccggcccgcgctcccaccccccgggccgtgtccttgctttccccccgtctccccccccgccttctcctcctcctcctcgtttttccaaaccccgcccacccggcccggcccggcccggcccggccaccgccgcccacccacccacctcgggatacccagccccggtcccccgttccccgggggccgttatctccagcgccccgtccggcgcgccgccccccgccgctaaaccccatcccgcccccgggaccccacatataagcccccagccacacgcaagaacagacacgcagaacggctacgaggaggaggaggagaaggcggggggggagacggggggaaagcaaggacacggcccggggggtgggagcgcgggccgggccgctcgtaagagccgcgacccggccgccggggagcgttgtcgccgtcggtctgccggcccccgtccctcccttttttgaccaaccagcgccctcccccccgcgcgggccgggccgctcgtaagagccgcgacccggccgccggggagcgttgtcgccgtcggtctgccggcccccgtccctcccttttttgaccaaccagcgccctcccccccaccaccattcctactaccaccaccaccaccacccccaccaccgacacctcccgcgcacccccgcccacatccccccaccccgcaccacgagcacggggtgggggtagcaggggatcaaaggggggcaaagccggcggggcggttcgggggggcgggagaccgagtaggcccgcccatacgcggcccctcccggcagccacgccccccagcgtcgggtgtcacggggaaagagcaggggagagggggggagaggggagagggggggagagggggtatataaaccaacgaaaagcgcgggaacggggatacggggcttgtgtggcacgacgtcgtggttgtgttactgggcaaacacttggggactgtaggtttctgtgggtgccgaccctaggcgctatggggattttgggttgggtcgggcttattgccgttggggttttgtgtgtgcgggggggcttgtcttcaaccgaatatgttattcggagtcgggtggctcgagaggtgggggatatattaaaggtgccttgtgtgccgctcccgtctgacgatcttgattggcgttacgagaccccctcggctataaactatgctttgatagacggtatatttttgcgttatcactgtcccggattggacacggtcttgtgggataggcatgcccagaaggcatattgggttaacccctttttatttgtggcgggttttctggaggacttgagtcaccccgcgtttcctgccaacacccaggaaacagaaacgcgcttggccctttataaagagatacgccaggcgctggacagtcgcaagcaggccgccagccacacacctgtgaaggctgggtgtgtgaactttgactattcgcgcacccgccgctgtgtagggcgacaggatttgggacctaccaacggaacgtctggacggaccccggttctgccgccggacgatgaagcgggcctgcaaccgaagcccctcaccacgccgccgcccatcatcgccacgtcggaccccaccccgcgacgggacgccgccacaaaaagcagacgccgacgaccccactcccggcgcctctaacgatgcctcgacggaaacccgtccgggttcggggggcgaaccggccgcctgtcgctcgtcagggccggcgggcgctcctcgccgccctagaggctgtcccgctggtgtgacgttttcctcgtccgcgccccccgaccctcccatggatttaacaaacgggggggtgtcgcctgcggcgacctcggcgcctctggactggaccacgtttcggcgtgtgtttctgatcgacgacgcgtggcggcccctgatggagcctgagctggcgaaccccttaaccgcccacctcctggccgaatataatcgtcggtgccagaccgaagaggtgctgccgccgcgggaggatgtgttttcgtggactcgttattgcacccccgacgaggtgcgcgtggttatcatcggccaggacccatatcaccaccccggccaggcgcacggacttgcgtttagcgtgcgcgcgaacgtgccgcctcccccgagtcttcggaatgtcttggtggccgtcaagaactgttatcccgaggcacggatgagcggccacggttgcctggaaaagtgggcgcgggacggcgtcctgttactaaacacgaccctgaccgtcaagcgcggggcggcggcgtcccactctagaatcggttgggaccgcttcgtgggcggagttatccgccggttggccgcgcgccgccccggcctggtgtttatgctctggggcgcacacgcccagaatgccatcaggccggaccctcgggtccattgcgtcctcaagttttcgcacccgtcgcccctctccaaggttccgttcggaacctgccagcatttcctcgtggcgaaccgatacctcgagacccggtcgatttcacccatcgactggtcggtttgaaaggcatcgacgtccggggtttttgtcggtgggggcttttgggtatttccgatgaataaagacggttaatggttaaacctctggtctcatacgggtcggtgatgtcgggcgtcgggggagagggagttccctctgcgcttgcgattctagcctcgtggggctggacgttcgacacgccaaaccacgagtcggggatatcgccagatacgactcccgcagattccattcggggggccgctgtggcctcacctaaccaacctttacacgggggcccggaacgggaggccacagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagcctccagatgtttgtgttgtgcaaacgcgcccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgccccccccccccccccgccccccccccccccccccccccccccctccccccccccccccccctcttcccccgtgacacccgacgctggggggcgtggctgccgggaggggccgcgtatgggcgggcctactcggtctcccgcccccccgaaccgccccgccggctttgccccccgcgatcttcggacgggcggctctgccccgccgtccccgtgttcgtccacgagttcgtctcgtccgagccaatgcgcctccaccgagataacgtcatgctggcctcgggggccgagtaaccgccccccccccatgccaccctcactgcccgtcgcgcgtgtttgatgttaataaataacacataaatttggctggttgtttgttgtctttaatggaccgcccgcaagggggggggggcgtttcagtgtcgggtgacgagcgcgatccggccgggatcctaggaccccaaaagtttgtctgcgtattccagggtggggctcagttgaatctcccgcagcacctctaccagcaggtccgcggtgggctggagaaactcggccgtcccggggcaggcggttgtcgggggtggaggcgcggcgcccaccccgtgtgccgcgcctggcgtctcctctgggggcgacccgtaaatggttgcagtgatgtaaatggtgtccgcggtccagaccacggtcaaaatgccggccgtggcgctccgggcgctttcgccgcgcgaggagctgacccaggagtcgaacggatacgcgtacatatgggcgtcccacccgcgttcgagcttctggttgctgtcccggcctataaagcggtaggcacaaaattcggcgcgacagtcgataatcaccaacagcccaatgggggtgtgctggataacaacgcctccgcgcggcaggcggtcctggcgctcccggccccgtaccatgatcgcgcgggtgccgtactcaaaaacatgcaccacctgcgcggcgtcgggcagtgcgctggtcagcgaggccctggcgtggcataggctatacgcgatggtcgtctgtggattggacatctcgcggtgggtagtgagtcccccgggccgggttcggtggaactgtaaggggacggcgggttaatagacaatgaccacgttcggatcgcgcagagccgatagtatgtgctcactaatgacgtcatcgcgctcgtggcgctcccggagcggatttaagttcatgcgaaggaattcggaggaggtggtgcgggacatggccacgtacgcgctgttgaggcgcaggttgccgggcgtaaagcagatggcgaccttgtccaggctaaggccctgggagcgcgtgatggtcatggcaagcttggagctgatgccgtagtcggcgtttatggccatggccagctccgtagagtcaatggactcgacaaactcgctgatgttggtgttgacgacggacatgaagccgtgttggtcccgcaagaccacgtaaggcaggggggcctcttccagtaactcggccacgttggccgtcgcgtgccgcctccgcagctcgtccgcaaaggcaaacacccgtgtgtacgtgtatcccatgagcgtataattgtccgtctgcagggcgacggacatcagccccccgcgcggcgagccggtcagcatctcgcagccccggaagataacgttgtccacgtacgtgctaaagggggcgacttcaaatgcctccccgaagagctcttggaggattcggaatctcccgaggaaggcccgcttcagcagcgcaaactgggtgtgaacggcggcggtggtctccggttccccgggggtgtagtggcagtaaaacacgtcgagctgttgttcgtccagccccgcgaaaataacgtcgaggtcgtcgtcgggaaaatcgtccgggcccccgtcccgcggccccagttgcttaaaatcaaacgcacgctcgccgggggcgcctgcgtcggccattaccgacgcctgcgtcggcacccccgaagatttggggcgcagagacagaatctccgccgttagttctcccatgcgggcgtacgcgagggtcctctgggtcgcatccaggcccgggcgctgcagaaagttgtaaaaggagataagcccgctaaatatgagccgcgacaggaacctgtaggcaaactccaccgaagtctccccctgagtctttacaaagctgtcgtcacgcaacactgcctcgaaggcccggaacgtcccactaaacccaaaaaccagttttcgcaggcgcgcggtcaccgcgatctggctgttgaggacgtaagtgacgtcgttgcgggccacgaccagctgctgtttgctgtgcacctcgcagcgcatgtgccccgcgtcctggtcctggctctgcgagtagttggtgatgcggctggtgttggccgtgagccacttttcaatagtcaggccgggctggtgtgtcagccgtcggtattcgtcaaactccttgaccgacacgaacgtaagcacggggagggttaacacgacgaactccccctcacgggtcaccttcaggtaggcgtggagcttggccatgtacgcgctcacctctttgtgggaggagaacagccgcgtccagccggggaggttggcggggttggtgatgtagttttccgggacgacgaagcgatccacgaactgcatgtgctcctcggtgatgggcaggccgtactccagcaccttcatgaggttaccgaactcgtgctcgacgcaccgtttgttgttaataaaaatggcccagctatacgagaggcgggcgtactcgcgcagcgtgcgggtgcagatgaggtacgtgagcacgttctcgctctggcggacggaacaccgcagtttctggtgctcgaaggcgactccagggacgccgtctgcgtcggcgagcccacacacaccaacacgggccgcaggcgggccgcgtactggggggtgtggtacagggcgttaatcatccaccagcaatacaccacggccgtgaggaggtgacgcccaaggagcccggcctcgtcgatgacgatcacgttgctgcgggtaaaggccggcagcgccccgtgggtggccggggccaaccgcgtcagggcgccctcggccaaccccagggtccgttccagggcggccagggcgcgaaactcgttccgcaactcctcgcccccggaggcggccagggcgcgcttcgtgaggtccaaaatcacctcccagtagtacgtcagatctcgtcgctgcaggtcctccagcgaggcggggttgctggtcagggtgtacgggtactgtcccagttgggcctggacgtgattcccgcgaaacccaaattcatgaaagatggtgttgatgggtcggctgagaaaggcgcccgagagtttggcgtacatgttttgggccgcaatgcgcgtggcgcccgtcaccacacagtccaagacctcgttgattgtctgcacgcacgtgctctttccggagccagcgttgccggtgataagatacaccgcgaacggaaactccctgaggggcaggcctgcgggggactctaaggccgccacgtcccggaaccactgcagacggggcacttgcgctccgtcgagctgttgttgcgagagctctcggatgcgcttaaggattggctgcaccccgtgcatagacgtaaaatttaaaaaggcctcggccctccctggaacggctggtcggtccccgggttgctgaaggtgcggcgggccgggtttctgtccgtctagctggcgctccccgccggccgccgccatgaccgcaccacgctcgtgggcccccactacgcgtgcgcggggggacacggaagcgctgtgctcccccgaggacggctgggtaaaggttcaccccacccccggtacgatgctgttccgtgagattctccacgggcagctggggtataccgagggccagggggtgtacaacgtcgtccggtccagcgaggcgaccacccggcagctgcaggcggcgatctttcacgcgctcctcaacgccaccacttaccgggacctcgaggcggactggctcggccacgtggcggcccgcggtctgcagccccaacggctggttcgccggtacaggaacgcccgggaggcggatatcgccggggtggccgagcgggtgttcgacacgtggcggaacacgcttaggacgacgctgctggactttgcccacgggttggtcgcctgctttgcgccgggcggcccgagcggcccgtcaagcttccccaaatatatcgactggctgacgtgcctggggctggtccccatattacgcaagcgacaagaagggggtgtgacgcagggtctgagggcgtttctcaagcagcacccgctgacccgccagctggccacggtcgcggaggccgcggagcgcgccggccccgggttttttgagctggcgctggccttcgactccacgcgcgtggcggactacgaccgcgtgtatatctactacaaccaccgccggggcgactggctcgtgcgagaccccatcagcgggcagcgcggagaatgtctggtgctgtggccccccttgtggaccggggaccgtctggtcttcgattcgcccgtccagcggctgtttcccgagatcgtcgcgtgtcactccctccgggaacacgcgcacgtctgccggctgcgcaataccgcgtccgtcaaggtgctgctggggcgcaagagcgacagcgagcgcggggtggccggtgccgcgcgggtcgttaacaaggtgttgggggaggacgacgagaccaaggccgggtcggccgcctcgcgcctcgtgcggcttatcatcaacatgaagggcatgcgccacgtaggcgacattaacgacaccgtgcgtgcctacctcgacgaggccggggggcacctgatagacgccccggccgtcgacggtaccctccctggattcggcaagggcggaaacaaccgcgggtctgcgggccaggaccagggggggcgggcgccgcagcttcgccaggccttccgcacggccgtggttaacaacatcaacggcgtgttggagggctatataaataacctgtttggaaccatcgagcgcctgcgcgagaccaacgcgggcctggcgacccaattgcaggagcgcgaccgcgagctccggcgcgcaacagcgggggccctggagcgccagcagcgcgcggccgacctggcggccgagtccgtgaccggtggatgcggcagccgccctgcgggggcggacctgctccgggccgactatgacattatcgacgtcagcaagtccatggacgacgacacgtacgtcgccaacagctttcagcacccgtacatcccttcgtacgcccaggacctggagcgcctgtcgcgcctctgggagcacgagctggtgcgctgttttaaaattctgtgtcaccgcaacaaccagggccaagagacgtcgatctcgtactccagcggggcgatcgccgcattcgtcgccccctactttgagtcagtgcttcgggccccccgggtaggcgcgcccatcacgggctccgatgtcatcctgggggaggaggagttatgggatgcggttttaaaaaacccgcctgcaaacgtacctgacagacatcgcggccctgttcgtcgcggacgtccagcacgcagcgctgcccccgcccccctccccggtcggcgccgatttccggcccggcgcgtccccgcggggccggtccagatcgcggtcgcccggaagaactgcgccaggcgcgccggaccagggcgggggcatcgggcaccgggatggccgccgcgacggccgacgatgaggggtcggccgccaccatcctcaagcaggccatcgccggggaccgcagcctggtcgaggcggccgaggcgattagccagcagacgctgctccgcctggcctgcgaggtgcgccaggtcggcgaccgccagccgcggtttaccgccaccagcatcgcgcgcgtcgacgtcgcgcctgggtgccggttgcggttcgttctggacgggagtcccgaggacgcctatgtgacgtcggaggattactttaagcgctgctgcggccagtccagttatcgcggcttcgcggtggcggtcctgacggccaacgaggaccacgtgcacagcctggccgtgccccccctcgttctgctgcaccggttctccctgttcaaccccagggacctcctggactttgagcttgcctgtctgctgatgtacctggagaactgcccccgaagccacgccaccccgtcgacctttgccaaggttctggcgtggctcggggtcgcgggtcgccgcacgtccccattcgaacgcgttcgctgccttttcctccgcagttgccactgggtcctaaacacactcatgttcatggtgcacgtaaaaccgttcgacgacgagttcgtcctgccccactggtacatggcccggtacctgctggccaacaacccgccccccgttctctcggccctgttctgtgccaccccgacgagctcctcattccggctgccggggccgcccccccgctccgactgcgtggcctataaccccgccgggatcatggggagctgctgggcgtcggaggaggtgcgcgcgcctctggtctattggtggctttcggagaccccaaaacgacagacgtcgtcgctgttttatcagttttgttgaattttaggaaataaacccggttttgtttctgtggcctcccgacggatgcgcgtgtccttcctccgtcttggtgggtgggtgtctgtgtatcgcgtcccatctgtgcggagagggggggcatgtcggcacgtattcggacagactcaagcacacacgggggagcgctcttgtctcagggcaatgtttttattggtcaaactcaggcaaacagaaacgacatcttgtcgtcaaagggatacacaaacttccccccctctccccatactcccgccagcaccccggtaaacaccaactcaatctcgcgcaggatttcgcgcaggtgatgagcgcagtccacgggggggagcacaaggggccgcgggtatagatcgacggggacgccgaccgactccccgcctccgggacagacacgcacgacgcgccgccagtagtgctctgcgtccagcaaggcgccgccgcggaaggcagtggggggcaaggggtcgctagcctcaaagggggacacccgaacgctccagtactccgcgtccaaccgtttattaaacgcgtccacgataaggcggtcgcaggcgtcctccataaggccccgggccgtgagtgcgtcctcctccggcacgcctgccgttgtcaggcccaggacccgtcgcagcgtgtcgcgtacgaccccggccgccgtggtgtacgcgggcccgcggagaggaaatcccccaagatggtcagtgttgtcgcgggagttccagaaccacactcccgcctggttccaggcgactgcgtgggtgtagacgccctcgagggccaggcacagtgggtgccgcagccggaggccgttggccctaagcacggctcccacggccgtctcgatggcccgccgggcgtcctcgatcaccccggaagccgcatccgcgtcttgggggtccacgttaaagacaccccagaacgcacccccatcgcccccgcagaccgcgaacttcaccgagctggccgtctcctcgatctgcaggcagacggcggccattaccccacccaggagctgccgcagcgcagggcaggcgtcgcacgtgtccgggaccaggcgctccaagacggccccggcccagggctctgagggagcggccaccaccagcgcgtccagtcttgctaggcccgtccggccgtgggggtccgccagcccgctccccccgaggtcggccagggccaccaggagctgggcgcgaagtccggggaagcaaaaccgcgccgtccagacgggcccgacggccgcgggcgggtctaacagttggatgattttagtggcgggatgccaccgcgccaccgcctcccgcaccgcgggcaggaggcatccggctgccgccgaggccacgccgggccaggctcgcggggggaggacgaccctggcccccaccgcgggccaggcccccaggagcgcggcgtaagcggccgcggccccgcgcaccaggtcccgtgccgactcggccgtggccggcacggtgaacgtgggccaacccggaaaccccaggacggcaaagtacgggacgggtcccccccggacctcaaactcgggccccagaaaggcaaagacgggggccagggccccgggggcggcgtggaccgtggtatgccactgccggaaaagggcgacgagcgccggcgcggagaacttctcgccggcgcttacaaagtagtcgtaatcgcggggcagcagcacccgtgccgtgactcgttgcgggtgcccgcgtggccgcaggcccacctcgcacacctcgaccaggtccccgaacgcgccctccttcttgatcggcggaaacgcaagagtctggtattcgcgcgcaaatagcgcggttccggtggtgatgttaacggtcagcgaagcggcggacgcgcactggggggtgtcgcgaatggccgccaggcgcgcccacgccagccgcgcgtcgggatgctcggcaacgcgcgccgccagggccatagggtcgatgtcaatgttggcctccgcgaccaggagagcggcgcgaggggcggcgggcgggccccacgacgctctctcaactttcaccaccagtcccgtgcgtgggtccgagccgatacgcagcggggcgaacagggccaccggcccggtctggcgctccagggccgccaggacgcacgcgtacagcgcccgccacagagtcgggttctccaggggctccagcggggaggcggccggcgtcgtcgcggcgcgggcggccgccacgacggcctggacggagacgtccgcggagccgtagaaatcccgcagctccgtcgcggtgacggagacctccgcaaagcgcgcgcgaccctcccctgcggcgttgcgacatacaaaatacaccagggcgtggaagtactcgcgagcgcgggggggcagccataccgcgtaaagggtaatggcgctgacgctctcctccacccacacgatatctgcggtgtccatcgcacggcccctaaggatcacgggcggtctgtgggtcccatgctgccgtgcctggccgggcccggtgggtcgcggaaaccggtgacggggggggggcggtttttggggttggggtgggggtgggaaacggcccgggtccgggggccaacttggcccctcggtgcgttccggcaacagcgccgccggtccgcggacgaccacgtaccgaacgagtgcggtcccgagacttatagggtgctaaagttcaccgccccctgcatcatgggccaggcctcggtggggagctccgacagcgccgcctccaggatgatgtcagcgttggggttggcgctggatgagtgcgtgcgcaaacagcgcccccacgcaggcacgcgtagcttgaagcgcgcgcccgcaaactcccgcttgtgggccataagcagggcgtacagctgcctgtgggtccggcaggcgctgtggtcgatgtggtgggcgtccaacaaccccacgattgtctgtttggtgaggtttttaacgcgccccgccccgggaaacgtctgcgtgcttttggccatctgcacgccaaacagttcgccccagattatcttgaacagcgccaccgcgtggtccgtctcgctaacggacccgcgcgggggacagccgcttagggcgtcggcgacgcgcttgacggcttcctccgagagcagaagtccgtcggttacgttacagtggcccagttcgaacaccagctgcatgtagcggtcgtagtggggggtcagtaggtccagcacgtcatcggggccgaaggtcctcccagatcccccggccgccgagtcccaatgcaggcgcgcggccatggtgctgcacaggcacaacagctcccagacgggggttacgttcagggtggggggcagggccacgagctccagctctccggtgacgttgatcgtggggatgacgcccgtggcgtagtggtcatagatccgccgaaatatggcgctgctgcgggtggccatgggaacgcggagacaggcctccagcaacgccaggtaaataaaccgcgtgcgtcccatcaggctgttgaggttgcgcatgagcgcgacaatttccgccggcgcgacatcggaccggaggtatttttcgacgaaaagacccacctcctccgtctcggcggcctgggccggcagcgacgcctcgggatcccggcaccgcagctcccgtagatcgcgctgggccctgagggcgtcgaaatgtacgccccgcaaaaacagacagaagtcctttggggtcagggtatcgtcgtgtccccagaagcgcacgcgtatgcagtttagggtcagcagcatgtgaaggatgttaaggctgtccgagagacacgccagcgtgcatctctcaaagtagtgtttgtaacggaatttgttgtagatgcgcgacccccgccccagcgacgtgtcgcatgccgacgcgtcacagcgccccttgaaccggcgacacagcaggtttgtgacctgggagaactgcgcgggccactggccgcaggaactgaccacgtgattaaggagcatgggcgtaaagacgggctccgagcgcgccccggagccgtccatgtaaatcagtagctcccccttgcggagggtgcgcacccgtcccagggactggtacacggacaccatgtccggtccgtagttcatgggttttacgtaggcgaacatgccatcaaagtgcaggggatcgaagctgaggcccacggttacgaccgtcgtgtatataaccacgcggtattggccccacgtggtcacgtccccgaggggggtgagcgagtgaagcaacagcacgcggtccgtaaactgacggcagaaccgggccacgatctccgcgaaggagaccgtcgacgaaaaaatgcagatgttatcgcccccgccaaggcgcgcttccagctccccaaagaacgtggccccccgggcgtccggagaggcgtccggagacgggccgctcggcggcccgggcgggcgcagggcagcctgcaggagctcggtccccagacgcgggagaaacaggcaccggcgcgccgaaaacccgggcatggcgtactcgccgaccaccacatgcacgtttttttcgccccggagaccgcacaggaagtccaccaactgcgcgttggcggttgcgtccatggcgatgatccgaggacaggtgcgcagcaggcgtagcattaacgcatccacgcggcccagttgctgcatcgttggcgaatagagctggcccagcgtcgacataacctcgtccagaacgaggacgtcgtagttgttcagaaggttggggcccacgcgatgaaggctttccacctggacgataagtcggtggaaggggcggtcgttcataatgtaattggtggatgagaagtaggtgacaaagtcgaccaggcctgactcagcgaaccgcgtcgccagggtctgggtaaaactccgacgacaggagacgacgagcacactcgtgtccggagagtggatcgcttcccgcagccagcggatcagcgcggtagtttttcccgaccccattggcgcgcggaccacagtcacgcacctggccgtcggggcgctcgcgttggggaaggtgacgggtccgtgctgctgccgctcgatcgttgttttcgggtgaacccggggcacccattcggccaaatcccccccgtacaacatccgcgctagcgatacgctcgacgtgtactgttcgcactcgtcgtccccaatgggacgcccggcccccagaggatctcccgactccgcgccccccacgaaaggcatgaccggggcgcggacggcgtggtgggtctggtgtgtgcaggtggcgacgtttgtggtctctgcggtctgcgtcacggggctcctcgtcctggcctctgtgttccgggcacggtttccctgcttttacgccacggcgagctcttatgccggggtgaactccacggccgaggtgcgcgggggtgtagccgtgcccctcaggttggacacgcagagccttgtgggcacttatgtaatcacggccgtgttgttgttggccgcggccgtgtatgccgtggtcggcgccgtgacctcccgctacgaccgcgccctggacgcgggccgccgtctggctgcggcccgcatggccatgccgcacgccacgctgatcgccggaaacgtctgctcttggttgctgcagatcaccgtcctgttgctggcccatcgcaccagccagctggcccacctggtttacgtcctgcactttgcgtgtctggtgtattttgcggcccatttttgcaccaggggggtcctgagcgggacgtatctgcgtcaggtgcacggcctgatggagccggccccgactcatcatcgcgtcgttggcccggctcgagccgtgctgacaaacgccttgctgttgggcgtcttcctgtgcacggccgacgccgcggtatccctgaataccatcgccgcgttcaactttaatttttcggccccgggcatgctcatatgcctgaccgtgctgttcgcccttctcgtcgtatcgctgttgttggtggtcgagggggtgttgtgtcactacgtgcgcgtgttggtgggcccccacctgggggccgtggccgccacgggcatcgtcggcctggcatgcgagcactattacaccaacggctactacgttgtggagacgcagtggccgggggcccagacgggagtccgcgtcgccctcgccctggtcgccgcctttgccctcggcatggccgtgctccgctgcacccgcgcctatctgtatcacaggcggcaccacaccaaattttttatgcgcatgcgcgacacgcgacaccgcgcacattccgccctcaagcgcgtacgcagttccatgcgcggatcgcgagacggccgccacaggcccgcacccggcagcccgcccgggattcccgaatatgcggaagacccctacgcgatctcatacggcggccagctcgaccggtacggagattccgacggggagccgatttacgacgaggtggcggacgaccaaaccgacgtattgtacgccaagatacaacacccgcggcacctgcccgacgacgagcccatctatgacaccgttggggggtacgaccccgagcccgccgaggaccccgtgtacagcaccgtccgccgttggtagctgtttggttccgttttaataaaccgtttgtgtttaacccgaccgtggtgtatgtctggtgtgtggcgtccgatcccgttactatcaccgttccccccaaaccccggcgattgtgggtttttttaaaaacgacacgcgtgcgaccgtatacagaacattgttgttttttattcgctatcggacatggggggtggaaactgggtggcggggcaggcgcctccgggggttcgccggtgagtgtggcgcgaggggggatccgacgaacgcaggcgctgtctccccggggcccgcgtaaccccgcgcatatccgggggcacgtagaaattaccttcctcttcggactcgatatccacgacgtcaaagtcgtgggcggtcagcgagacgacctccccgtcgtcggtgatgaggacgttgtttcggcagcagcagggccgggtcccggagaacgagaggcccatagctcggcgagcgtgtcgtcgaacgccaggcggctgcttcgctgtatggccttatagatctccggatcgatgcggacgggggtaatgatcagggcgatcggaacggcctggttcgggagaatggacgccttgctgggtcctgcggccccgagagccccggcgccgtcctccaggcggaacgttacgccctcctccgcgctagtgcggtgcctgccgataaacgtcaccagatgcgggtggggggggcagtcggggaagtggctgtcgagcacgtagccctgcaccaagatctgcttaaagttcgggtgacgggggttcgcgaagacgggctcgcggcgtaccagatccccggagctccaggacacgggggagatggtgtggcgtccgaggtcgggggtgccaaacagaagcacctccgagacaacgccgctatttaactccaccaaggcccgatccgcggcggagcaccgccttttttcgcccgaggcgtgggcctctgaccaggcctggtcttgcgtgacgagagcctcctccgggccggggacgcgcccgggcgcgaagtatcgcacgctgggcttcgggatcgaccggataaatgcccggaacgcctccggggaccggtgtgccatcaagtcctcgtacgcggaggccgtggggtcgctggggtccatggggtcgaaagcgtacttggcccggcatttgacctcgtaaaaggccaggggggtcttggggactggggccaagtagccgtgaatgtcccgaggacagacgagaatatccagggacgccccgaccatccccgtgtgaccgtccatgaggaccccacacgtatgcacgttctcttcggcgaggtcgccgggttcgtggaagataaagcgccgcgtgtcggcgccggcctcgccgccgtcgtccgcgcggcccacgcagtagcgaaacagcaggcttcgggccgtcggctcgttcacccgcccgaacatcaccgccgaagactgtacatccggccgcaggctggcgttgtgcttcagccactggggcgagaaacacggaccctgggggccccagcggagggtggatgcggtcgtgaggccccgccggagcagggcccatagctggcagtcggcctggttttgcgtggccgcctcgtaaaaccccatgaggggccggggcgccacggcgtccgcggcggccgggggcccgcggcgcgtcaggcgccataggtgccggccgagtccgcggtccaccatacccgcctcctcgaggaccacggccagggaacacagataatccaggcgggcccagaggggaccgatggccagaggggcgcggacgccgcgcagcaacccgcgcaggggcgctcgaacgtctcggctagtatatgggagggcagcgcgttggggatcaccgacgccgaccacatagagtcaaggtccggggagtcgggatcggcgtccgggtcgcgggcgtgggtgcccccaggagatagcggaatgtctggggtcggaggccctgaggcgtcagaaagtgccggcgacgcggcccggggcttttcgtctgcggtgtcggtggcgtgctgatcacgtggggggttaacgggcgaatgggagctcgggtccacaactgacgtcgtctggggtggggggggcaggggacggaaggtggttgttagcggaagactgttagggcgggggcgcttgggggggctgtcggggccacgaggggtgtcctcggccagggcccaggaacgcttagtcacggtgcgtcccggcggacatgctgggcctcccgtggactccatttccgagacgacgtggggggagcggtggttgagcgcgccgccgggtgaacgctgattctcacgacagcgcgtgccgcgcgcacgggttggtgtgacacaggcgggacaccagcaccaggagaggcttaagctcgggaggcagcgccaccgacgacagtatcgccttgtgtgtgtgctggtaatttatacaccgatccgtaaacgcgcgccgaatcttgggattgcggaggtggcgccggatgccctctgggacgtcatacgccaggccgtgggtgttggtctcggccgagttgacaaacagggctgggtgcagcacgcggcgataggcgagcagggccagggcgaagtccagcgacagctggttgttgaaatactggtaaccgggaaaccgggtcacgggtacgcccaggctcggggcgacgtacacgctaaccaccaactccagcagcgtctggccaagggcgtacaggtcaaccgctaacccgacgtcgtgcttcaggcggtggttggtaaattcggcccgttcgttgttaaggtatttcaccaacagctccgggggctggttatacccgtgacccaccagggtgtgaaagttggctgtggttagggcggtgggcatgccaaacatccggggggacttgaggtccggctcctggaggcaaaactgcccccgggcgatcgtggagttggagttgagggtgacgaggctaaagtcggcgaggacggcccgccggagcgagacggcgtccgaccgcagcatgacgaggatgttggcgcacttgatatccaggtggctgatcccgcaggtggtgtttaaaaacacaacggcgcgggccagctccgtgaagcactggtggagggccgtcgagaccgaggggtttgttgtgcgcagggacgccagttggccgatatacttaccgaggtccatgtcgtacgcggggaacactatctgtcgttgttgcagcgagaacccgaggggcgcgatgaagccgcggatgttgtgggtgcggccggcgcgtagagcgcactccccgaccaacagggtcgcgatgagctcaacggcaaaccactccttttcctttatggtcttaacggcaagcttatgttcgcgaatcagttggacgtcgccgtatcccccagaccccccgaagcttcgggccccggggatctcgagggtcgtgtagtgtagggcggggttgatggcgaacacggggctgcatagcttgcggatgcgcgtgagggtaaggatgtgcgagggggacgaggggggtgcggttaacgccgcctgggatctgcgcaggggcgggcggttcagtttggccgccgtaccgggcgtctcgggggacgcgcggcgatgagacgagcggctcattcgccatcgggatagtcccgcgcgaagccgctcgcggaggccggatcggtggcgggacccgtgggaggagcgggagccggcggcgtcctggagagaggggccgctggggcgcccggaggccccgtgtgggttggagtgtatgtaggatgcgagccaatccttgaaggactgttggcgtgcaccttgggggctgaggttagctgccacatgaccagcaggtcgctgtctgcgggactcatccatccttcggccaggtcgccgtcttcccacagagaagcgttggtcgctgcttcctcgagttgctcctcctggtccgcaagacgatcgtccacggcgtccaggcgctcaccaagcgccggatcgaggtaccgtcggtgtgcggttagaaagtcacgacgcgccgcttgctcctccacgcgaattttaacacaggtcgcgcgctgtcgcatcatctctaagcgcgcgcgggactttagccgcgcctccaattccaagtgggccgcctttgcagccataaaggcgccaacaaaccgaggatcttgggtgctgacgccctcccggtgcagctgcagggtctggtccttgtaaatctcggctcggaggtgcgtctcggccaggcgtcggcgcagggccgcgtgggcggcatctcggtccattccgccaccctgcgggcgacccggggggtgctctgatagtctcgcgtgcccaaggcccgtgatcggggtacttcgccgccgcgacccgccacccggtgtgcgcgatgtttggtcagcagctggcgtccgacgtccagcagtacctggagcgcctcgagaaacagaggcaacttaaggtgggcgcggacgaggcgtcggcgggcctcacaatgggcggcgatgccctacgagtgccctttttagatttcgcgaccgcgacccccaagcgccaccagaccgtggtcccgggcgtcgggacgctccacgactgctgcgagcactcgccgctcttctcggccgtggcgcggcggctgctgtttaatagcctggtgccggcgcaactaaaggggcgtgatttcgggggcgaccacacggccaagctggaattcctggcccccgagttggtacgggcggtggcgcgactgcggtttaaggagtgcgcgccggcggacgtggtgcctcagcgtaacgcctactatagcgttctgaacacgtttcaggccctccaccgctccgaagcctttcgccagctggtgcactttgtgcgggactttgcccagctgcttaaaacctccttccgggcctccagcctcacggagaccacgggccccccaaaaaaacgggccaaggtggacgtggccacccacggccggacgtacggcacgctggagctgttccaaaaaatgatccttatgcacgccacctactttctggccgccgtgctcctcggggaccacgcggagcaggtcaacacgttcctgcgtctcgtgtttgagatccccctgtttagcgacgcggccgtgcgccacttccgccagcgcgccaccgtgtttctcgtcccccggcgccacggcaagacctggtttctagtgcccctcatcgcgctgtcgctggcctcctttcgggggatcaagatcggctacacggcgcacatccgcaaggcgaccgagccggtgtttgaggagatcgacgcctgcctgcggggctggttcggttcggcccgagtggaccacgttaaaggggaaaccatctccttctcgtttccggacgggtcgcgcagtaccatcgtgtttgcctccagccacaacacaaacgtaagtcctcttttctttcgcatggctctcccaaggggccccgggtcgacccgacccacacccacccacccacccacatacacacacaaccagacgcgggaggaaagtctgccccgtgggcactgatttttattcgggatcgcttgaggaggcccgggcaacggcccgggcaacggtggggcaactcgtagcaaataggcgactgatgtacgaagagaagacacacaggcgccacccggcgctggtcggggggatgttgtccgcgccgcaccgtcccccgacgacctcttgcagacggtccgtgatgcaaggacggcggggggcctgcagcagggtgaccgtatccacgggatggccaaagagaagcggacacaggctagcatccccctggaccgccagggtacactgggccatcttggcccacagacacggggcgacgcagggacaggactccgttacgacggaggagagccacagtgcgttggcggaatcgatgtggggcggcggggcgcaggactcgcagccccccgggtggttggtgatcctggccaggagccatcccagatggcgggccctgcttcccggtggacagagcgaccccaggtcgctgtccatggcccagcagtagatctggccgctggggaggtgccaccaggcccccgggcccaaggcgcaacacgcgcccggctccgggggggtcttcgcggggaccagatacgcgccatccagctcgccgaccactggctcctccgcgagctgttcggtggttgggtcgggggtttcctccgggggggtggccgcccgtatgcgggcgaacgtgagggtgcacaggagcggggtcagggggtgcgtcacgctccggaggtggacgatcgcgcagtagcggcgctcgcggttaaagaaaaagagggcaaagaaggtgttcgggggcaaccgcagcgccttggggcgcgtcagatacagaaaaatctcgcagaagagggcgcgcccggggtctgggttaggaagggccacctgacacagaggctcggtgaggaccgttagacaccgaaagatcttgagccgctcgtccgcccgaacgacgcgccacacaaagacggagttgacaatgcgcgcgatagagtcgacgtccgtccccaggtcgtcgactctgtcgcgcgtgccgcgagctccggcccgggaatccggccggggcaaggtccccgggggaccaggcggcgccaggggccgccggggtcccagctgcgccatgccgggggcggggggagggcaaaccccagaggcgggggccaacggcgcggggaggagtgggtgggcgaggtggccgggggaaggcgcccgctagcgagaacggccgttcccggacgacaccttgcgacaaaacctaaggacagcggcccgcgcgacggggtccgagaggctaaggtaggccgcgatgttaatggtgaacgcaaagccgccgggaaagacaactatgccacagaggcggcgattaaaccccaggcagaggtaggcgtagctttccccgggcaggtattgctcgcagaccctgcgtggggctgtggaggggacggcctccatgaagcgacatttactctgctcgcgtttactgacgtcaccatccatcgccacggcgattggacgattgttaagccgcagcgtgtctccgcttgtgctgtagtagtcaaaaacgtaatggccgtcggagtcggcaaagcgggccgggaggtcgtcgccgagcgggacgacccgccgcccccgaccgccccgtccccccaggtgtgccaggacggccagggcatacgcggtgtgaaaaaaggcgtcgggggcggtcccctcgacggcgcgcatcaggttctcgaggagaatggggaagcgcctggtcacctcccccagccacgcgcgttggtcggggccaaagtcatagcgcaggcgctgtgagattcgagggccgccctgaagcgcggcccggatggcctggcccagggcccggaggcacgccagatgtatgcgcgcagtaaaggcgacctcggcggcgatgtcaaagggcggcaggacggggcgcgggtggcgcaggggcacctcgagcgcgggaaagcggagcagcagctccgcctgcccagcgggagacagctggtgggggcgcacgacgcgttctgcggcgcaggcctcggtcagggccgtggccagcgccgaggacagcagcgggggcgggcgcgtcgcccgccccacgccacggagttctcgtaggagacgacgacgaagcgctgcttggttccgtagtggtggcgcaggaccacggagatagaacgacggctccacagccagtccggccggtcgccgccggccagggcttcccatccgcgatccaaccactcgaccagcgaccgcggctttgcggtaccaggggtcagggttagaacgtcgttcaggatgtcctcgcccccgggcccgtggggcactggggccacaaagcggcccccgcctgggggctccagacccgccaacaccgcatctgcgtcagccgcccccatggcgcccccgctgacggcctggtgaaccagggcgccctggcggagccccgatgcaacgccacaggccgcacgcccggtccgagcgcggaccgggtggcggcgggtgacgtcctgcactgcccgctgaaccaacgcgaggatctcctcgttctcctgcgcgatggacacgtcctgggccgcggtcgtgtcgccgccgggggccgtcagctgctcctccggggagatgggggggtcggacgccccgacgatgggcgggtctgcgggcgcccccgcgtggggccgggccaagggctgcggacgcggggacgcgctttcccccagacccatggacaggtgggccgcagcctccttcgcggccggcggggcggcggcgccaagcagagcgacgtagcggcacaaatgccgacagacgcgcatgatgcgcgtgctgtcggccgcgtagcgcgtgttgggggggacgagctcgtcgtaactaaacagaatcacgcgggcacagctcgcccccgagccccacgcaaggcgcagcgccgccacggcgtacgggtcatagacgccctgtgcgtcacacaccacgggcaaggagacgaacaaccccccggcgctggacgcacgcggaaggaggccagggtgtgccggcacgacgggggccagaagctcccccaccgcatccgcgggcacgtaggcggcaaacgccgtgcaccacggggtacagtcgccggtggcatgagcccgagtctggatttcgacctggaagtttgcggccgtcccgagtccggggcggccgcgcatcagggcggccagagggattcccgcggccgccaggcactcgctggatatgatgacgtgaaccaaagacgagggccgacccgggacgtggccgagatcgtactggacctcgttggccaagtgcgcgttcatggttcgggggtgggtgtgggtgtgtaggcgatgcgggtcccccgagtccgcgggaagggcgcgggtttggcgcgcgtatgcgtattcgccaacggaggcgtgcgtgcttatgcgcggcgcgtttcttctgtctccagggaatccgaggccaggactttaacctgctctttgtcgacgaggccaactttattcgcccggatgcggtccagacgattatgggctttctcaaccaggccaactgcaagattatcttcgtgtcgtccaccaacaccgggaaggccagtacgagctttttgtacaacctccgcggggccgccgacgagcttctcaacgtggtgacctatatatgcgatgatcacatgccgcgggtggtgacgcacacaaacgccacggcctgttcttgttatatcctcaacaagcccgttttcatcacgatggacggggcggttcgccggaccgccgatttgtttctggccgattccttcatgcaggagatcatcgggggccaggccagggagaccggcgacgaccggcccgttctgaccaagtctgcgggggagcggtttctgttgtaccgcccctcgaccaccaccaacagcggcctcatggcccccgatttgtacgtgtacgtggatcccgcgttcacggccaacacccgagcctccgggaccggcgtcgctgtcgtcgggcggtaccgcgacgattatatcatcttcgccctggagcacttttttctccgcgcgctcacgggctcggcccccgccgacatcgcccgctgcgtcgtccacagtctgacgcaggtcctggccctgcatcccggggcgtttcgcggcgtccgggtggcggtcgagggaaatagcagccaggactcggccgtcgccatcgccacgcacgtgcacacagagatgcaccgcctactggcctcggagggggccgacgcgggctcgggccccgagcttctcttctaccactgcgagcctcccgggagcgcggtgctgtacccctttttcctgctcaacaaacagaagacgcccgcctttgaacactttattaaaaagtttaactccgggggcgtcatggcctcccaggagatcgtttccgcgacggtgcgcctgcagaccgacccggtcgagtatctgctcgagcagctgaataacctcaccgaaaccgtctcccccaacacggacgtccgtacgtattccggaaaacggaacggcgcctcggatgaccttatggtcgccgtcattatggccatctaccttgcggcccaggccggacctccgcacacattcgctcccatcacacgcgtttcgtgagcgcccaataaacacacccaggtatgctacgcacgaccacggtgtcgcctgttaagggggggggaagggggtgttggcgggaagcgtgggaacacgggggattctctcacgaccggcaccagtaccacccccctgtgaacacagaaaccccaacccaaatcccataaacatacgacacacaggcatattttggaatttcttaggtttttatttatttaggtatgctggggtttctccctggatgcccacccccacccccccccgtgggtctagccgggccttagggatagcgtataacgggggccatgtctccggaccgcacaacggccgcgccgtcaaaggtgcacacccgaaccacgggagccagggccaaggtgtctcctagttggcccgcgtgggtcagccaggcgacgagcgcctcgtagagcggcagccttcgctctccatcctgcatcagggccggggcttcggggtgaatgagctgggcggcctcccgcgtgacactctgcatctgcaggagagcgttcacgtacccgtcctgggcacttagcgcaaagagccgggggattagcgtaaggatgatggtggttccctccgtgatcgagtaaaccatgttaaggaccagcgatcgcagctcggcgtttacggggccgagttgttggacgtccgccagcagcgagaggcgactcccgttgtagtacagcacgttgaggtctggcagccctccggggtttctggggctggggttcaggtcccggatgcccctggccacgagccgcgccacgatttcgcgcgccaggggcgatggaagcggaacgggaaaccgcaacgtgaggtccagcgaatccaggcgcacgtccgtcgcttggccctcgaacacgggcgggacgaggctgatggggtccccgttacagagatctacgggggaggtgttgcgaaggttaacggtgccggcgtgggtgaggcccacgtccagggggcaggcgacgattcgcgtgggaagcacccgggtgatgaccgcggggaagcgccttcggtacgccagcaacagccccaacgtgtcgggactgacgcctccggagacgaaggattcgtgcgccacgtcggccagcgtcagttgccggcggatggtcggcaggaataccacccgcccttcgcagcgctgcagcgccgccgcatcggggcgcgagatgcccgagggtatcgcgatgtcagtttcaaagccgtccgccagcatggcgccgatccacgcggcagggagtgcagtggtggttcgggtggcgggaggagcgcggtgggggtcagcggcgtagcagagacgggcgaccaacctcgcataggacggggggtgggtcttagggggttgggaggcgacagggaccccagagcatgcgcggggaggtctgtcgggcccagacgcaccgagagcgaatccgtccatggagtcccggcctgggttttatggggcccggccctcggaatcgcggcttgtcggcggggacaaagggggcggggctagggggcttgcggaaacagaagacgtgtgggataaaagaatcgcactaccccaaggaagggcggggcggtttattacagagccagtcccttgagcggggatgcgtcatagacgagatactgcgcgaagtgggtctcccgcgcgtgggcttccccgttgcgggcgctgcggaggagggcggggtcgctggcgcaggtgagcgggtaggcctcctgaaacaggccacacgggtcctccacgagttcgcggcaccccggggggcgcttaaactgtacgtcgctggcggcggtggccgtggacaccgccgaacccgtctccacgatcaggcgctccaggcagcgatgtttggcggcgatgtcggccgacgtaaagaacttaaagcaggggctgagcaccggcgaggccccgttgaggtggtaggccccgttatagagcaggtccccgtacgaaaatcgctgcgacgcccacgggttggccgtggccgcgaaggcccgggacgggtcgctctggccgtggtcgtacatgagggcggtgacatccccctccttgtcccccgcgtaaacgcccccggcggcgcgtccccgggggttgcagggccggcggaagtagttgacgtcggtcgacacgggggtggcgataactcacacacggcgtcctggccgtggtccatccctgcgcgccgcggcacctgggcgcccccgaacacggggacgggctgggccggccccaggcggtttcccgccacgaccgcgttccgcaggtacacggctgccgcgttgtccagtagagggggagccccgcggcccaggtaaaagttttggggaaggttgcccatgtcggtgacggggttgcggacggttgccgtggccacgacggcggtgtagcccacgcccaggtccacgttcccgcgcggctgggtgagcgtgaagtttacccccccgccagtttcatgccgggccacctggagctggcccaggaagtacgcctccgacgcgcgctccgagaacagcacgttctcagtcacaaagcggtcctgtcggacgacggtgaacccaaacccgggatggaggcccgtcttgagctgatgatgcaaggccacgggactgatcttgaagtaccccgccatgagcgcgtaggtcagcgcgttctccccggccgcgctctcgcggacgtgctgcacgacgggctgtcggatcgacgaaaagtagttggcccccagagccggggggaccagggggacctgccgcgacaggtcgcgcagggccggggggaaattgggcgcgttcgccacgtggtcggccccggcgaacagcgcgtggacggggagggggtaaaaatagtcgccattttggatggtatggtccagatgctggggggccatcagcaggattccggcgtgcaacgccccgtcgaatatgcgcatgttggtggtggacgcggtgttggcgcccgcgtcgggcgccgccgagcagagcagcgccgttgtgcgttcggccatgttgtgggccagcacctgcagcgtgagcatggcgggcccgtccactaccacgcgcccgttgtgaaacatggcgttgaccgtgttggccaccagattggccggggcagggggtgcgcggggccgtcacggggcgctggggcaatcctcgccgggggtgatctccgggaccaccatgttctgcagggtggcgtatacgcggtcgaagcgaacccccgcggtgcagcagcggccccgcgagaaggcgggcaccatcacgtagtagtaaatcttgtggtgcacggtccagtccgccccccggtgcggccggtcgtccgcggcgtccgcggctcgggcctgggtgttgtgcagcagctggccgtcgttgcggttgaagtccgcggtcgccacgttacacgccgctgcgtacacggggtcgtggccccccgcgctaacccggcagtcgcgatggcggtccagggccgcgcgccgcatcagggcgtcgcagtcccacacgaggggtggcagcagcgccgggtctcgcattaggtgattcagttcggcttgcgcctgcccgcccagttccgggccggtcagggtaaagtcatcaaccagctgggccagggcctcgacgtgcgccaccaggtcccggtacacggccatgcactcctcgggaaggtctcccccgaggtaggtcacgacgtacgagaccagcgagtagtcgttcacgaacgccgcgcaccgcgtgttgttccagtagctggtgatgcactggaccacgagccgggccagggcgcagaagacgtgctcgctgccgtgtatggcggcctgcagcaggtaaaacaccgccgggtagttgcggtcttcgaacgccccgcgaacggcggcgatggtggcgggggctggcgtggcgtcccacccccagctccaggccccgggcgtcccggaacgccgccggacatagcgccaggggcaagttgccgttcaccacgcgccaggtggcctggatctcccccgggccggccgggggaacgtccccccccggcagctccacgtcggccacccccacgaagaagtcgaacgcggggtgcagctcaagagccaggttggcgttgtcgggctgcataaactgctccggggtcatctggccttccgcgacccatcggacccgcccgtgggccaggcgctgcccccaggcgttcaaaaacagctgctgcatgtctgcggcggggccggccggggccgccacgtacgccccgtacggattggcggcttcgacggggtcgcggttaaggcccccgaccgccgcgtcaacgttcatcagcgaagggtggcacacggtcccgatcgcgtgttccagagacaggcgcagcacctggcggtccttcccccaaaaaaacagctggcggggcgggaaggcgcggggatccgggtggccgggggcggggctaggtccccggcgtgcgcggcaaaccgttccatgaccggattgaacaggcccaggggcaggacgaacgtcaggtccatggcgcccaccagggggtagggaacgttggtggcggcgtagatgcgcttctccagggcctccaaaaagatcagcttctcgccgatggacaccagatccgcgcgcacgcgcgtcgtctggggggcgctctcgagctcgtccagcgtctgccggttcaggtcgagctgctcctcctgcatctccagcaggtggcggcccacgtcgtccagacttcgcacggccttgcccatcacgagcgccgtgaccaggttggccccgttcaggaccatctcgccgtacgtcaccggcacgtcggcttcggtgtcctccactttcaggaaggactgcaggaggcgctgtttgatcggggcggtggtgacgagcaccccgtcgaccggacgcccgcgcgtgtcggcatgcgtcagacggggcacggccacggagggctgcgtggccgtggtgaggtccacgagccaggcctcgacggcctcccggcggtggcccgccttgcccaggaaaaagctcgtctcgcagaagcttcgctttagctcggcgaccagggtcgcccgggccaccctggtggccaggcggccgttgtccaggtatcgttgcatcggcaacaacaaagccaggggcggcgccttttccagcagcacgtgcagcatctggtcggccgtgccgcgctcaaacgccccgaggacggcctggacgttgcgagcgagctgttggatggcgcgcaactggcgatgcgcgctgatacccgtcccgtccagggcctcccccgtgagcagggcgatggcctcggtggccaggctgaaggcggcgttcagggcccggcggtcgataatcttggtcatgtaattgtgtgtgggttgctcgatggggtgcgggccgtcgcgggcaatcagcggctggtggacctcgaactgtacgcgcccctcgttcatgtaggccagctccggaaacttggtacacacgcacgccaccgacaacccgagctccagaaagcgcacgagcgacagggtgttgcaatacgaccccaacagggcgtcgaactcgacgtcatacaggctgtttgcatcggagcgcacgcgggaaaaaaaatcgaacaggcgtcgatgcgacgccacctcgatcgtgctaaggagggacccggtcggcaccatggccgcggcataccggtatcccggagggtcgcggttgggagcggccatggggtcgcgtggagatcggctgtctctagcgatattggcccggggaggctaagatccaccccaacgcccggccacccgtgtacgtgcccgacggcccaaggtccaccgaaagacacgacgggcccggacccaaaaaggcgggggatgctgtgtgagaggccgggtgtcggtcgggggggaaaggcaccgggagaaggctgcggcctcgttccaggagaacccagtgtccccaacagacccggggacgtgggatccaagcttgatctctatcactgatagggagatctctatcactgatagggatcccaggccttatataccccccccgcccccccccgttagaacgcgacgggtgcattcaagatggccctggtccaaaagcgtgccaggaagaaattggcagaggcggcaaagctgtccgccgccgccacccacatcgaggccccggccgcgcaggctatccccagggcccgtgtgcgcaggggatcggtgggcggcagcatttggttggtggcgataaagtggaaaagcccgtccggactgaaggtctcgtgggcggcggcgaacaaggcacacagggccgtgcctcccaaaaacacggacatcccccaaaacacgggcgccgacaacggcagacgatccctcttgatgttaacgtacaggaggagcgcccgcaccgcccacgtaacgtagtagccgacgatggcggccaggatacaggccggcgccaccacccttccggtcagcccgtaatacatgcccgctgccaccatctccaacggcttcaggaccaaaaacgaccaaaggaacagaatcacgcgctttgaaaagaccggctgggtatggggcggaagacgcgagtatgccgaactgacaaaaaagtcagaggtgccgtacgaggacaatgaaaactgttcctccagtggcagttctccctcctcccccccaaaggcggcctcgtcgaccagatctcgatccaccagaggaaggtcatcccgcatggtcatggggtgtgcggtggaggtggggagaccgaaaccgcaaagggtcgcttacgtcagcaggatcccgagatcaaagacacccgggttcttgcacaaacaccacccgggttgcatccgcggaggcgagtgttttgataaggccgttccgcgccttgatataacctttgatgttgaccacaaaacccggaatttacgcctacgccccaatgcccacgcaagatgaggtaggtaaccccccccccgtgggtgtgacgttgcgtttagttcattggaggccaaggggaaaatggggtggggaggaaacggaaaacccagtaggccgtgttgggaacacgcccggggttgtcctcaaaaggcagggtccatactacggaagccgtcgttgtattcgagacctgcctgtgcgacgcacgtcggggttgcctgtgtccggttcggccccaccgcgtgcggcacgcacgaggacgagtccgcgtgctttattggcgttccaagcgttgccctccagtttctgttgtcggtgttcccccatacccacgcccacatccaccgtagggggcctctgggccgtgtcacgtcgccgcccgcgatggagcttagctacgccaccaccatgcactaccgggacgttgtgttttacgtcacaacggaccgaaaccgggcctactttgtgtgcggggggtgtgtttattccgtggggcggccgtgtgcctcgcagcccggggagattgccaagtttggtctggtcgttcgagggacaggcccagacgaccgcgtggtcgccaactatgtacgaagcgagctccgacaacgcggcctgcaggacgtgcgtcccattggggaggacgaggtgtttctggacagcgtgtgtcttctaaacccgaacgtgagctccgagctggatgtgattaacacgaacgacgtggaagtgctggacgaatgtctggccgagtactgcacctcgctgcgaaccagcccgggtgtgctaatatccgggctgcgcgtgcgggcgcaggacagaatcatcgagttgtttgaacacccaacgatagtcaacgtttcctcgcactttgtgtataccccgtccccatacgtgttcgccctggcccaggcgcacctcccccggctcccgagctcgctggaggccctggtgagcggcctgtttgacggcatccccgccccacgccagccacttgacgcccacaacccgcgcacggatgtggttatcacgggccgccgcgccccacgacccatcgccgggtcgggggcggggtcggggggcgcgggcgccaagcgggccaccgtcagcgagttcgtgcaagtcaaacacattgaccgcgtgggccccgctggcgtttcgccggcgcctccgccaaacaacaccgactcaagttccctggtgcccggggcccaggattccgccccgcccggccccacgctaagggagctgtggtgggtgttttatgccacagaccgggcgctggaggagccccgcgccgactctggcctcacccgcgaggaggtacgtgccgtacgtgggttccgggagcaggcgtggaaactgtttggctccgcgggggccccgcgggcgtttatcggggccgcgttgggcctgagccccctccaaaagctagccgtttactactatatcatccaccgagagaggcgcctgtcccccttccccgcgctagtccggctcgtaggccggtacacacagcgccacggcctgtacgtccctcggcccgacgacccagtcttggccgatgccatcaacgggctgtttcgcgacgcgctggcggccggaaccacagccgagcagctcctcatgttcgaccttctccccccaaaggacgtgccggtgggaagcgacgtgcaggccgacagcaccgctctgctgcgctttatagaatcgcaacgtctcgccgtccccgggggggtgatctcccccgagcacgtcgcgtaccttggtgcgttcctgagcgtgctgtacgctggccgcgggcgcatgtccgcagccacgcacaccgcgcggctgacaggggtgacctccctggtgctagcggtgggtgacgtggaccgtctttccgcgtttgaccgcggagcggcgggcgcggccagccgcacgcgggccgccgggtacctggatgtgcttcttaccgttcgtctcgctcgctcccaacacggacagtctgtgtaacagaccccaataaacgtatgtcgctaccacacccttgtgtgtcaatggacgcctctccgggggggaagggaaaacaaagaggggctgggggagcggcaccaccggggcctgaacaaacaaaccacagacacggttacagtttattcggtcgggcggagaaacggccgaagccacgccccctttattcgcgtctccaaaaaaacgggacacttgtccggagaacctttaggatgccagccagggcggcggtaatcataaccacgcccagcgcagaggcggccagaaacccgggcgcaattgcggccacgggctgcgtgtcaaaggctagcaaatgaatgacggttccgtttggaaatagcaacaaggccgtggacggcacgtcgctcgaaaacacgcttggggcgccctccgtcggcccggcggcgatttgctgctgtgtgttgtccgtatccaccagcaacacagacatgacctccccggccggggtgtagcgcataaacacggcccccacgagccccaggtcgcgctggttttgggtgcgcaccagccgcttggactcgatatcccgggtggagccttcgcatgtcgcggtgaggtaggttaggaacagtgggcgtcggacgtcgacgccggtgagcttgtagccgatcccccggggcagaggggagtgggtgacgacgtagctggcgttgtgggtgatgggtaccaggatccgtggctcgacgttggcagactgccccccgcaccgatgtgaggcctcagggacgaaggcgcggatcagggcgttgtagtgtgcccagcgcgtcagggtcgaggcgaggccgtgggtctgctgggccaggacttcgaccggggtctcggatcgggtggcttgagccagcgcgtccaggataaacacgctctcgtctagatcaaagcgcagggaggccgcgcatggcgaaaagtggtccggaagccaaaagagggttttctggtggtcggcccgggccagcgcggtccggaggtcggcgttggtcgctgcggcgacgtcggacgtacacagggccgaggctatcagaaggctccggcgggcgcgttcccgctgcaccgccgaggggacgcccgccaagaacggctgccggaggacagccgaggcgtaaaatagcgcccggtggacgaccggggtggtcagcacgcggccccctagaaactcggcatacagggcgtcgatgagatgggctgcgctgggcgccactgcgtcgtacgccgaggggctatccagcacgaaggccagctgatagcccagcgcgtgtaatgccaagctctgttcgcgctccagaatctcggccaccaggtgctggagccgagcctctagctgcaggcgggccgtgggatccaagactgacacattaaaaaacacagaatccgcggcacagcccgcggccccgcgggcggccaacccggcaagcgcgcgcgagtgggccaaaaagcctagcaggtcggagaggcagaccgcgccgtttgcgtgggcggcgttcacgaaagcaaaacccgacgtcgcgagcagccccgttaggcgccagaagagagggggacgcgggccctgctcggcgcccgcgtcccccgagaaaaactccgcgtatgcccgcgacaggaactgggcgtagttcgtgccctcctccgggtagccgcccacgcggcggagggcgtccagcgcggagccgttgtcggcccgcgtcagggaccctaggacaaagacccgataccgggggccgcccgggggcccgggaagagcccccggggggttttcgtccgcggggtccccgacccgatctagcgtctggcccgcggggaccaccatcacttccaccggagggctgtcgtgcatggatatcacgagccccatgaattcccgcccgtagcgcgcgcgcaccagcgcggcatcgcacccgagcaccagctcccccgtcgtccagatgcccacgggccacgtcgaggccgacggggagaaatacacgtacctacctggggatctcaacaggccccgggtggccaaccaggtcgtggacgcgttgtgcaggtgcgtgatgtccagctccgtcgtcgggtgccgccgggccccaaccggcggtcgggggggcggtgtatcacgcggcccgctcgggtggctcgccgtcgccacgttgtctccccgcgggaacgtcagggcctcggggtcagggacggccgaaaacgttacccaggcccgggaacgcagcaacacggaggcggttggattgtgcaagagacccttaaggggggcgaccgcggggggaggctgggcggtcggctcgaccgtgatgggggcgggcaggctcgcgttcgggggccggccgagcaggtaggtcttcgagatgtaaagcagctggccggggtcccgcggaaactcggccgtggtgaccaatacaaaacaaaagcgctcctcgtaccagcgaagaaggggcagagatgccgtagtcaggtttagttcgtccggcggcgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaagaataacccccacgaaccataaaccattccccatgggggaccccgtccctaacccacggggcccgtggctatggcagggcttgccgccccgacgttggctgcgagccctgggccttcacccgaacttgggggttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttatttccgtcatagcgcgggttccttccggtattgtctccttccgtgtttcagttagcctcccccatctcccgggcaaacgtgcgcgccaggtcgcagatcgtcggtatggagcctggggtggtgacgtgggtctggaccatcccggaggtaagttgcagcagggcgtcccggcagccggcgggcgattggtcgtaatccaggataaagacgtgcatgggacggaggcgtttggccaagacgtccaaggcccaggcaaacacgttatacaggtcgccgttgggggccagcaactcgggggcccgaaacagggtaaataacgtgtccccgatatggggtcgtgggcccgcgttgctctggggctcggcaccctggggcggcacggccgtccccgaaagctgtccccaatcctcccgccacgacccgccgccctgcagataccgcaccgtattggcaagcagcccgtaaacgcggcgaatcgcggccaacatagccaggtcaagccgctcgccggggcgctggcgtttggccaggcggtcgatgtgtctgtcctccggaagggcccccaacacgatgtttgtgccgggcaaggtcggcgggatgagggccacgaacaccagcacggcctggggggtcatgctgcccataaggtatcgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccagcacctgccagtaagtcatcggctcgggtacgtagacgatatcgtcgcgcgaacccagggccaccagcagttgcgtggtggtggttttccccatcccgtgaggaccgtctatataaacccgcagtagcgtgggcattttctgctccaggcggacttccgtggcttcttgctgccggcgagggcgcaacgccgtacgtcggttgctatggccgcgagaacgcgcagcctggtcgaacgcagacgcgtgttgatggcaggggtacgaagccatacgcgcttctacaaggcgcttgccaaagaggtgcgggagtttcacgccaccaagatctgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagctcctgcagtccctcgcgcctccgggtgacaagatagtgtacctgtgccccgtcctggtgtttgtcgcccaacggacgctccgcgtcagccgcgtgacccggctcgtcccgcagaaggtctccggtaatatcaccgcagtcgtgcggatgctccagagcctgtccacgtatacggtccccatggagcctaggacccagcgagcccgtcgccgccgcggcggcgctgcccgggggtctgcgagcagaccgaaaaggtcaccctctggggcacgcgacccgcccgggccagcggcccgccaggtaccacccgccgaccaaacccccgcctccacggagggcgggggggtgcttaagaggatcgcggcgctcttctgcgtgcccgtggccaccaagaccaaaccccgagctgcctccgaatgagagtgtttcgttccttccccctccccccgcgtcagacaaaccctaaccaccgcttaagcggcccccgcgaggtccgaagactcatttggatccggcgggagccacctgacaacagcccccgggtttccccacgccagacgccggtccgctgtgccatcgctccccttcatcccacccccatcttgtccccaaataaaacaaggtctggtagttaggacaacgaccgcagttctcgtgtgttattgtcgctctccgcctctcgcagatggacccgtattgcccatttgacgctctggacgtctgggaacacaggcgcttcatagtcgccgattcccgaaacttcatcacccccgagttcccccgggacttttggatgtcgcccgtctttaacctcccccgggagacggcggcggagcaggtggtcgtcctgcaggcccagcgcacagcggctgccgctgccctggagaacgccgccatgcaggcggccgagctccccgtcgatatcgagcgccggttacgcccgatcgaacggaacgtgcacgagatcgcaggcgccctggaggcgctggagacggcggcggccgccgccgaagaggcggatgccgcgcgcggggatgagccggcgggtgggggcgacgggggggcgcccccgggtctggccgtcgcggagatggaggtccagatcgtgcgcaacgacccgccgctacgatacgacaccaacctccccgtggatctgctacatatggtgtacgcgggccgcggggcgaccggctcgtcgggggtggtgttcgggacctggtaccgcactatccaggaccgcaccatcacggactttcccctgaccacccgcagtgccgactttcgggacggccggatgtccaagaccttcatgacggcgctggtcctgtccctgcagtcgtgcggccggctgtatgtgggccagcgccactattccgccttcgagtgcgccgtgttgtgtctctacctgctgtaccgaaacacgcacggggccgccgacgatagcgaccgcgctccggtcacgttcggggatctgctgggccggctgccccgctacctggcgtgcctggccgcggtgatcgggaccgagggcggccggccacagtaccgctaccgcgacgacaagctccccaagacgcagttcgcggccggcgggggccgctacgaacacggagcgctggcgtcgcacatcgtgatcgccacgctgatgcaccacggggtgctcccggcggccccgggggacgtcccccgggacgcgagcacccacgttaaccccgacggcgtggcgcaccacgacgacataaaccgcgccgccgccgcgttcctcagccggggccacaacctattcctgtgggaggaccagactctgctgcgggcaaccgcgaacaccataacggccctgggcgttatccagcggctcctcgcgaacggcaacgtgtacgcggaccgcctcaacaaccgcctgcagctgggcatgctgatccccggagccgtcccttcggaggccatcgcccgtggggcctccgggtccgactcgggggccatcaagagcggagacaacaatctggaggcgctatgtgccaattacgtgcttccgctgtaccgggccgacccggcggtcgagctgacccagctgtttcccggcctggccgccctgtgtcttgacgcccaggcggggcggccggtcgggtcgacgcggcgggtggtggtatgtcatcgggggcccgccaggcggcgctgggcgcctcaccgccctggaactcatcaaccgcacccgcacaaaccccacccccgtgggggaggttatccacgcccacgacgccctggcgatccaatacgaacaggggcttggcctgctggcgcagcaggcacgcattggcttgggctccaacaccaagcgtttctccgcgttcaacgttagcagcgactacgacatgttgtactttttatgtctggggttcattccacagtacctgtcggcggtttagtgggtggtgggcgaggggggagggggcattagggagaaagaacaagagcctccgttgggttttctttgtgcctgtactcaaaaggtcataccccgtaaacggcgggctccagtcccggcccggcggttggcgtgaacgcaacggcgggagctgggttagcgtttagtttagcattcgctctcgcctttccgcccgcccccgaccgttgagccttttttttttttttttttttttcgtccaccaaagtctctgtgggtgcgcgcatggcagccgatgccccgggagaccggatggaggagcccctgccagacagggccgtgcccatttacgtggctgggtttttggccctgtatgacagcggggactcgggcgagttggcattggatccggatacggtgcgggcggccctgcctccggataacccactcccgattaacgtggaccaccgcgctggctgcgaggtggggcgggtgctggccgtggtcgacgacccccgcgggccgttttttgtggggctgatcgcctgcggcaactggagcgcgtcctcgagacggccgccagcgctgcgattttcgagcgccgcgggccgccgctctcccgggaggagcgcctgttgtacctgatcaccaactacctgccctcggtctccctggccacaaaacgcctggggggcgaggcgcaccccgatcgcacgctgttcgcgcacgtagcgctgtgcgcgatcgggcggcgccttggcactatcgtcacctacgacaccggtctcgacgccgccatcgcgccctttcgccacctgtcgccggcgtctcgcgagggggcgcggcgactggccgccgaggccgagctcgcgctatccggacgcacctgggcgcccggcgtggaggcgctgcccacccgctgctttccaccgccgttaacaacatgatgctgcgggaccgctggagcctggtggccgagcggcggcggcaggccgggatcgccggacacacctacctccaggcgagcgaaaaattcaaaatgtggggggcggagcctgtttccgcgccggcgcgcgggtataagaacggggccccggagtccacggacataccgcccggctcgatcgctgccgcgccgcagggtgaccggtgcccaatcgtccgtcagcgcggggtcgcctcgcccccggtactgccccccatgaaccccgttccaacatcgggcaccccggcccccgcgccgcccggcgacgggagctacctgtggatcccggcctcccattacaaccagctcgtcgccggccacgccgcgccccaaccccagccgcattccgcgtttggtttcccggctgcggcgggggccgtggcctatgggcctcacggcgcgggtctttcccagcattaccctccccacgtcgcccatcagtatcccggggtgctgttctcgggacccagcccactcgaggcgcagatagccgcgttggtgggggccatagccgcggaccgccaggcgggcggtcagccggccgcgggagaccctggggtccgggggtcgggaaagcgtcgccggtacgaggcggggccgtcggagtcctactgcgaccaggacgaaccggacgcggactacccgtactaccccggggaggctcgaggcgggccgcgcggggtcgactctcggcgcgcggcccgccagtctcccgggaccaacgagaccatcacggcgctgatgggggcggtgacgtctctgcagcaggaactggcgcacatgcgggctcggaccagcgccccctatggaatgtacacgccggtggcgcactatcgccctcaggtgggggagccggaaccaacaacgacccacccggccctttgtcccccggaggccgtgtatcgccccccaccacacagcgccccctacggtcctccccagggtccggcgtcccatgcccccactcccccgtatgccccagctgcctgcccgccaggcccgccaccgcccccatgtccttccacccagacgcgcgcccctctaccgacggagcccgcgttcccccccgccgccaccggatcccaaccggaggcatccaacgcggaggccggggcccttgtcaacgccagcagcgcagcacacgtggacgttgacacggcccgcgccgccgatttgttcgtctctcagatgatgggggcccgctgattcgccccggtctttggtaccatgggatgtcttactgtatatctttttaaataaaccaggtaataccaaataagacccattggtgtatgttcttttttttttattgggaggggcgggtaggcgggtagctttacaatgcaaaagcctttgacgtggaggaaggcgtgggggggaggaaatcggcactgaccaagggggtccgttttgtcacgggaaaggaagaggaaaaggcgcggcacccgggggagttatgtgttcctttttctttcttcccacacacacacaaaaggcgtaccaaacaaaaaaaccaaaagatgcgcatgcggtttaacacccgtggtttttatttacaacaaaccccccgtcacaggtcgtcctcgtcggcgtcaccgtctttgttgggaacttgggtgtagttggtgttgcggcgcttgcgcatgaccatgtcggtgaccttggcgctgagcagcgcgctcgtgcccttcttcttggccttgtgttccgtgcgctccatggccgacaccagggccatgtaccgtatcatctccctggcctcggctagcttggcctcgtcaaagtcgccgccctcctcgccctccccggacgcgtccgggttggtggggttcttgagctccttggtggttagagggtacagggccttcatggggttgctctgcagccgcatgacgtaacgaaaggcgaagaaggccgccgccaggccggccaggaccaacagacccacggccagcgccccaaaggggttggacatgaaggaggacacgcccgacacggccgataccacgccgcccacgatgcccatcaccaccttgccgaccgcgcgccccaggtcgcccatcccctcgaagaacgcgcccaggcccgcgaacatggcggcgttggcgtcggcgtggatgaccgtgtcgatgtcggcgaagcgcaggtcgtgcagctggttgcggcgctggacctccgtgtagtccagcaggccgctgtccttgatctcgtggcgggtgtacacctccagggggacaaactcgtgatcctccagcatggtgatgttgaggtcgatgaaggtgctgacggtggtgatgtcggcgcggctcagctggtgggagtacgcgtactcctcgaagtacacgtagcccccaccgaaggtgaagtagcgccggtgtcccacggtgcacggctcgatcgcatcgcgcgtcagccgcagctcgttgttctcccccagctgcccctcgaccaacgggccctggtcttcgtaccgaaagctgaccagggggcggctgtagcaggccccgggccgcgagctgatgcgcatcgagttttggacgatcacgttgtccgcggcgaccggcacgcacgtggagacggccatcacgtcgccgagcatccgcgcgctcacccgccggcccacggtgaccgaggcgatggcgttggggttcagcttgcgggcctcgttccacagggtcagctcgtgattctgtagctcgcaccacgcgatggcaacgcggcccaacatatcgttgacatggcgctgtatgtggttgtacgtaaactgcagccgggcgaactcgatggaggaggtggtcttgatgcgctccacggacgcgttggcgctggccccgggcggcgggggcgtggggtttgggggcttgcggctctgctctcggaggtgttcccgcacgtacagctccgcgagcgtgttgctgagaaggggctggtacgcgatcagaaagcccccattggccaggtagtactgcggctggcccaccttgatgtgcgtcgcgttgtacctgcgggcgaagatgcggtccatggcgtcgcgggcgtccttgccgatgcagtcccccaggtccacgcgcgagagcgggtactcggtcaggttggtggtgaaggtggtggatatggcgtcggaggagaatcggaaggagccgccgtactcggagcgcagcatctcgtccacttcctgccacttggtcatggtgcagaccgacgggcgctttggcacccagtcccaggccacggtgaacttgggggtcgtgagcaggttccgggtggtcggcgccgtggcccgggccttggtggtgaggtcgcgcgcgtagaagccgtcgacctgcttgaagcggtcggcggcgtagctggtgtgttcggtgtgcgacccctcccggtagccgtaaaacggggacatgtacacaaagtcgccagtcgccagcacaaactcgtcgtacgggtacaccgagcgcgcgtccacctcctcgacgatgcagtttaccgtcgtcccgtaccggtggaacgcctccacccgcgaggggttgtacttgaggtcggtggtgtgccagccccggctcgtgcgggtcgcggcgttggccggtttcagctccatgtcggtctcgtggtcgtcccggtgaaacgcggtggtctccaggttgttgcgcacgtacttggccgtggaccgacagacccccttggcgttgatcttgtcgatcacctcctcgaaggggacgggggcgcggtcctcaaagatccccataaactgggagtagcggtggccgaaccacacctgcgaaacggtgacgtctttgtagtacatggtggccttgaacttgtacggggcgatgttctccttgaagaccaccgcgatgccctccgtgtagttctgaccctcgggccgggtcgggcagcggcgcggctgctcgaactgcaccaccgtggcgcccgtggggggtgggcacacgtaaaagtttgcatcggtgttctccgccttgatgtcccgcaggtgctcgcgcagggtggcgtggcccgcggcgacggtcgcgcccccttggcgttgatcttgtcgatcacctcctcgaaggggacgggggcgcggtcctcaaagatccccataaactgggagtagcggtggccgaaccacacctgcgaaacggtgacgtctttgtagtacatggtggccttgaacttgtacggggcgatgttctccttgaagaccaccgcgatgccctccgtgtagttctgaccctcgggccgggtcgggcagcggcgcggctgctcgaactgcaccaccgtggcgcccgtggggggtgggcacacgtaaaagtttgcatcggtgttctccgccttgatgtcccgcaggtgctcgcgcagggtggcgtggcccgcggcgacggtcgcgttgtcgccggcggggcgggggcgttgggttttcggtttttgttcttcttcggtttcgggtcccccgttggggcggcgccaagggcgggcggcgccggagtggcagggcccccgttcgccgcctgggtcgcggccgcgaccccaggcgtgccgggggaactcggagccgccgacgccaccaggacccccagcgtcaaccccaagagcgcccatacgacgaaccaccggcgcccccacgagggggcgccctggtgcatggcgggactacgggggcccgtcgtgccccccgtcaggtagcctgggggcgaggtgctggaggaccgagtagaggatcgagaaaacgtcgcggtcgtagaccacgaccgaccgggggccgatacagccgtcgggggcgctctcgacgatggccaccagcggacagtcggagtcgtacgtgagatatacgccgggcgggtaacggtaacgaccttcggaggtcgggcggctgcagtccgggcggcgcaactcgagctccccgcaccggtagaccgaggcaaagagtgtggtggcgataatcagctcgcgaatatatcgccaggcggcgcgctgagtgggcgttattccggaaatgccgtcaaaacagtaaaacctctgaaattcgctgacggcccaatcagcacccgagccccccgcccccatgatgaaccgggcgagctcctccttcaggtgcggcaggagccccacgttctcgacgctgtaatacagcgcggtgttggggggctgggcgaagctgtgggtggagtgatcaaagaggggcccgttgacgagctcgaagaagcgatgggtgatgctggggagcagggccgggtccacctggtgtcgcaggagagacgctcgcatgaaccggtgcgcgtcgaacacgcccggcgccgagcggttgtcgatgaccgtgcccgcgcccgccgtcagggcgcagaagcgcgcgcgcgccgcaaagccgttggcgaccgcggcgaacgtcgcgggcagcacctcgccgtggacgctgacccgcagcatcttctcgagctccccgcgctgctcgcggacgcagcgccccaggctggccaacgaccgcttcgtcaggcggtccgcgtacagccgccgtcgctcccgtacgtccgcggccgcttgcgtggcgatgtccccccacgtctcgggcccctgccccccgggcccgcggcgacggtcttcgtcctcgcccccgcccccgggagctcccaacccccgtgccccttcctctacggcgacacggtccccgtcgtcgtcggggcccgcgccgcccttgggcgcgtccgccgcgccccccgcccccatgcgcgccagcacgcgacgcagcgcctcctcgtcgcactgttcggggctgacgaggcgccgcaagagcggcgtcgtcaggtggtggtcgtagcacgcgcggatgagcgcctcgatctgatcgtcgggtgacgtggcctgaccgccgattattagggcgtccaccatatccagcgccgccaggtggctcccgaacgcgcgatcgaaatgctccgcccgccgcccgaacagcgccagttccacggccaccgcggcggtctcctgctgcaactcgcgccgcgccagcgcggtcaggttgctggcaaacgcgtccatggtggtctggccggcgcggtcgccggacgcgagccagaatcgcaattcgctgatggcgtacaggccgggcgtggtggcctgaaacacgtcgtgcgcctccagcagggcgtcggcctccttgcggaccgagtcgttctcgggcgacgggtggggctgcccgtcgccccccgcggtccgggccagcgcatggtccaacacggagagcgcccgcgcgcggtcggcgtccgacagcccggcggcgtggggcaggtaccgccgcagctcgttggcgtccagccgcacctgcgcctgctgggtgacgtggttacagatacggtccgccaggcggcgggcgatcgtcgccccctggttcgccgtcacacacagttcctcgaaacagaccgcgcaggggtgggacgggtcgctaagctccggggggacgataaggcccgaccccaccgcccccaccataaactcccgaacgcgctccagcgcggcggtggcgccgcgcgagggggtgatgaggtggcagtagtttagctgctttagaaagttctcgacgtcgtgcaggaaacacagctccatatggacggtcccgccatacgtatccagcctgacccgttggtgatacggacagggtcgggccaggcccatggtctcggtgaaaaacgccgcgacgtctcccgcggtcgcgaacgtctccaggctgcccaggagccgctcgccctcgcgccacgcgtactctagcagcaactccagggtgaccgacagcggggtgagaaaggccccggcctgggcctccaggcccggcctcagacgacgccgcagcgcccgcacctgaagcgcgttcagcttcagttgggggagcttcccccgtccgatgtgggggtcgcaccgccggagcagctctatctgaaacacataggtctgcacctgcccgagcagggctaacaacttttgacgggccacggtgggctcggacaccggggcggccatctcgcggcgccgatctgtaccgcggccggagtatgcggtggaccgaggcggtccgtacgctacccggtgtctggctgagccccggggtccccctcttcggggcggcctcccgcgggcccgccgaccggcaagccgggagtcggcggcgcgtgcgtttctgttctattcccagacaccgcggagaggaatcgcggcccgcccagagatatagacacggaacacaaacaagcacggatgtcgtagcaataatttattttacacacatccccgccccgccctaggttcccccaccccccaacccctcacagcatatccaacgtcaggtctccctttttgtcggggggcccctccccaaacgggtcatccccgtggaacgcccgtttgcggccggcaaatgccggtcccggggcccccgggccgccgaacggcgtcgcgttgtcgtcctcgcagccaaaatccccaaagttaaacacctccccggcgttgccgagttggctgactagggcctcggcctcgtgcgccacctccagggccgcgtccgtcgaccactcgccgttgccgcgctccagggcacgcgcggtcagctccatcatctcctcgcttaggtactcgtcctccaggagcgccagccagtcctcgatctgcagctgctgggtgcggggccccaggcttttcacggtcgccacgaacacgctactggcgacggccgccccgccctcggagataatgccccggagctgctcgcacagcgagctttcgtgcgctccgccgccgaggttcgaggccgcgcacacaaacccggcccggggacaggccaggacgaacttgcgggtgcggtcaaaaataaggagcgggcacgcgtttttgccgcccatcaggctggcccagttcccggcctgaaacacacggtcgttgccggccatgccgtagtatttgctgatgctcaaccccaacacgaccatggggcgcgccgccatgacgggccgcagcaggttgcagctggcgaacatggacgtccacgcgcccggatgcgcgtccacggcgtccatcagcgcgcgggccccggcctccaggcccgccccgccctgcgcggaccacgcggccgccgcctgcacgctggggggacggcgggaccccgcgatgatggccgtgagggtgttgatgaagtatgtcgagtgatcgcagtaccgcagaatctggtttgccatgtagtacatcgccagctcgctcacgttgttgggggccaggttaataaagtttatcgcgccgtagtccagggaaaactttttaatgaacgcgatggtctcgatgtcctcgcgcgacaggagccgggcgggaagctggttgcgttggagggccgtccagaaccactgcgggttcggctggttggaccccgggggcttgccgttggggaagatggccgcgtggaactgcttcagcagaaagcccagcggtccgaggaggatgtccacgcgcttgtcgggcttctggtaggcgctctggaggctggcgacccgcgccttggcggcctcggacgcgttggcgctcgcgcccgcgaacaacacgcggctcttgacgcgcagctccttgggaaaccccagggtcacgcgggcaacgtcgccctcgaagctgctctcggcgggggccgtctggccggccgttaggctgggggcgcagatagccgccccctccgagagcgcgaccgtcagcgttttggccgacagaaacccgttgttaaacatgtccatcacgcgccgccgcagcaccggttggaattgattgcgaaagttgcgcccctcgaccgactgcccggcgaacaccccgtggcactggctcagggccaggtcctgatacacggcgaggttggatcgccgcccgagaagctgaagcagggggcatggcccgcacgcgtacgggtccagcgtcagggacatggcgtggttggcctcgcccagaccgtcgcgaaacttgaagttcctcccctccaccaggttgcgcatcagctgctccacctcgcggtccacgacctgcctgacgttgttcaccaccgtatgcagggcctcgcggttggtgatgatggtctccagccgccccatggccgtggggaccgcctggtccacgtactgcagggtctcgagttcggccatgacgcgctcggtcgccgcgcggtacgtctcctgcatgatggtccgggcggtctcggatccgtccgcgcgcttcagggccgagaaggcggcgtagtttcccagcacgtcgcagtcgctgtacatgctgttcatggtcccgaagacgccgatggctccgcgggcggcgctggcgaacttgggatggcgcgcccggaggcgcatgagcgtcgtgtgtacgcaggcgtggcgcgtgtcgaaggtgcacaggttgcagggcacgtcggtctggttggagtccgcgacgtatcgaaacacgtccatctcctggcgcccgacgatcacgccgccgtcgcagcgctccaggtaaaacagcatcttggccagcagcgccggggaaaacccacacagcatggccaggtgctcgccggcaaattcctgggttccgccgacgaggggcgcggtgggccgaccctcgaacccgggcaccacgtgtccctcgcggtccacctgtgggttggccgccacgtgggtcccgggcacgaggaagaagcggtaaaaggagggtttgctgtggtcctttgggtccgccggaccggcgtcgtccacctcggtgagatggagggccgagttggtgctaaataccatggcccccacgagtcccgcggcgcgcgccaggtacgccccgacggcgttggcgcgggccgcggccgtgtcctggccctcgcacagcggccacgcggagatgtcggtgggcggctcgtcgaagacggccatcgacacgatagactcgagggccagggcggcgtctccggccatgacggaggccaggcgctgttcgaacccgcccgccgggcccttgccgccgccgtcgcgcccaccccgcggggtcttaccctggctggcttcgaaggccgtgaacgtaatgtcggcggggagggcggcgccctcgtggttttcgtcaaacgccaggtgggcggccgcgcgggccacggcgtccacgtttcggcatcgcagtgccacggcggcgggtcccacgaccgcctcgaacaggaggcggttgagggggcggttaaaaaacggaagcgggtaggtaaaattctccccgatcgatcggtggttggcgttgaacggctcggcgatgacccggctaaaatccggcatgaacagctgcaacggatacacgggtatgcggtgcacctccgccccgcctatggttaccttgtccgagcctcccaggtgcagaaaggtgttgttgatgcacacggcctccttgaagccctcggtaacgaccagatacaggagggcgcggtccgggtccaggccgaggcgctcacacagcgcctcccccgtcgtctcgtgtttgaggtcgccgggccggggggtgtagtccgaaaagccaaaatggcggcgtgcccgctcgcagagtcgcgtcaggtttggggcctgggtgctggggtccaggtgccggccgccgtgaaagacgtacacggacgagctgtagtgcgatggcgtcagtttcagggacaccgcggtacccccgagccccgtcgtgcgagaacccacgaccacggctacgttggcctcaaagccgctctccacggtcaggcccacgaccaggggcgccacggcgacgtcggcatcgccgctgcgcgccgacagtaacgccagaagctcgatgccttcggacggacacgcgcgagcgtacacgtatcccaggggcccgggggggaccttgatggtggttgccgtcttgggctttgtctccatgtcctcctggcaatcggtccgcaaacggaggtaatcccggcacgacgacggacgcccgacgaggtatgtctcccgagcgtcaaaatccggggggggggggggggggggggcggcgacggtcaaggggagggtgggagaccggggttggggaatgaatccctacccttcacagacaacccccgggtaaccacggggtgccgatgaaccccggcggctggcaacgcggggtccctgcgagaggcacagatgcttacggtcaggtgctccgggccgggtgcgtctgatatgcggttggtatatgtacactttacctgggggcgtgccggaccgccccagcccctcccacaccccgcgcgtcatcagccggtgggcgtggccgctattataaaaaaagtgagaacgcgaagcgttcgcactttgtcctaataatatatatattattaggacaaagtgcgaacgcttcgcgttctcactttttttataatagcggccacgcccaccggctacgtcacgctcctgtcggccgccggcggtccataagcccggccggccgggccgacgcgaataaaccgggccgccggccggggcgccgcgcagcagctcgccgcccggatccgccagacaaacaaggcccttgcacatgccggcccgggcgagcctgggggtccggtaattttgccatcccacccaagcggcttttggggtttttcctcttcccccctccccacatcccccctctttaggggttcgggtgggaacaaccgcgatgttttccggtggcggcggcccgctgtcccccggaggaaagtcggcggccagggcggcgtccgggttttttgcgcccgccggccctcgcggagccggccggggacccccgccttgtttgaggcaaaacttttacaacccctacctcgccccagtcgggacgcaacagaagccgaccgggccaacccagcgccatacgtactatagcgaatgcgatgaatttcgattcatcgccccgcgggtgctggacgaggatgcccccccggagaagcgcgccggggtgcacgacggtcacctcaagcgcgcccccaaggtgtactgcgggggggacgagcgcgacgtcctccgcgtcgggtcgggcggcttctggccgcggcgctcgcgcctgtggggcggcgtggaccacgccccggcggggttcaaccccaccgtcaccgtctttcacgtgtacgacatcctggagaacgtggagcacgcgtacggcatgcgcgcggcccagttccacgcgcggtttatggacgccatcacaccgacggggaccgtcatcacgctcctgggcctgactccggaaggccaccgggtggccgttcacgtttacggcacgcggcagtacttttacatgaacaaggaggaggttgacaggcacctacaatgccgcgccccacgagatctctgcgagcgcatggccgcggccctgcgcgagtccccgggcgcgtcgttccgcggcatctccgcggaccacttcgaggcggaggtggtggagcgcaccgacgtgtactactacgagacgcgccccgctctgttttaccgcgtctacgtccgaagcgggcgcgtgctgtcgtacctgtgcgacaacttctgcccggccatcaagaagtacgagggtggggtcgacgccaccacccggttcatcctggacaaccccgggttcgtcaccttcggctggtaccgtctcaaaccgggccggaacaacacgctagcccagccgcgggccccgatggccttcgggacatccagcgacgtcgagtttaactgtacggcggacaacctggccatcgaggggggcatgagcgacctaccggcatacaagctcatgtgcttcgatatcgaatgcaaggcggggggggaggacgagctggcctttccggtggccgggcacccggaggacctggttattcagatatcctgtctgctctacgacctgtccaccaccgccctggagcacgtcctcctgttttcgctcggttcctgcgacctccccgaatcccacctgaacgagctggcggccaggggcctgcccacgcccgtggttctggaattcgacagcgaattcgagatgctgttggccttcatgacccttgtgaaacagtacggccccgagttcgtgaccgggtacaacatcatcaacttcgactggcccttcttgctggccaagctgacggacatttacaaggtccccctggacgggtacggccgcatgaacggccggggcatgtttcgcgtgtgggacataggccagagccacttccagaagcgcagcaagataaaggtgaacggcatggtgaacatcgacatgtacgggatcataaccgacaagatcaagctctcgagctacaagctcaacgccgtggccgaagccgtcctgaaggacaagaagaaggacctgagctatcgcgacatccccgcctactacgccaccgggcccgcgcaacgcggggtgatcggcgagtactgcatacaggattccctgctggtgggccagctgttttttaagtttttgccccatctggagctctcggccgtcgcgcgcttggcgggtattaacatcacccgcaccatctacgacggccagcagatccgcgtctttacgtgcctgctgcgcctggccgaccagaagggctttattctgccggacacccaggggcgatttaggggcgccgggggggaggcgcccaagcgtccggccgcagcccgggaggacgaggagcggccagaggaggagggggaggacgaggacgaacgcgaggagggcgggggcgagcgggagccggagggcgcgcgggagaccgccggccggcacgtggggtaccagggggccagggtccttgaccccacttccgggtttcacgtgaaccccgtggtggtgttcgactttgccagcctgtaccccagcatcatccaggcccacaacctgtgcttcagcacgctctccctgagggccgacgcagtggcgcacctggaggcgggcaaggactacctggagatcgaggtgggggggcgacggctgttcttcgtcaaggctcacgtgcgagagagcctcctcagcatcctcctgcgggactggctcgccatgcgaaagcagatccgctcgcggattccccagagcagccccgaggaggccgtgctcctggacaagcagcaggccgccatcaaggtcgtgtgtaactcggtgtacgggttcacgggagtgcagcacggactcctgccgtgcctgcacgttgccgcgacggtgacgaccatcggccgcgagatgctgctcgcgacccgcgagtacgtccacgcgcgctgggcggccttcgaacagctcctggccgatttcccggaggcggccgacatgcgcgcccccgggccctattccatgcgcatcatctacggggacacggactccatatttgtgctgtgccgcggcctcacggccgccgggctgacggccatgggcgacaagatggcgagccacatctcgcgcgcgctgtttctgccccccatcaaactcgagtgcgaaaagacgttcaccaagctgctgctgatcgccaagaaaaagtacatcggcgtcatctacgggggtaagatgctcatcaagggcgtggatctggtgcgcaaaaacaactgcgcgtttatcaaccgcacctccagggccctggtcgacctgctgttttacgacgataccgtatccggagcggccgccgcgttagccgagcgccccgcagaggagtggctggcgcgacccctgcccgagggactgcaggcgttcggggccgtcctcgtagacgcccatcggcgcatcaccgacccggagagggacatccaggactttgtcctcaccgccgaactgagcagacacccgcgcgcgtacaccaacaagcgcctggcccacctgacggtgtattacaagctcatggcccgccgcgcgcaggtcccgtccatcaaggaccggatcccgtacgtgatcgtggcccagacccgcgaggtagaggagacggtcgcgcggctggccgccctccgcgagctagacgccgccgccccaggggacgagcccgccccccccgcggccctgccctccccgaccaagcgcccccgggagacgccgtcgcatgccgaccccccgggaggcgcgtccaagccccgcaagctgctggtgtccgagctggccgaggatcccgcatacgccattgcccacggcgtcgccctgaacacggactattacttctcccacctgttgggggcggcgtgcgtgacattcaaggccctgtttgggaataacgccaagatcaccgagagtctgttaaaaaggtttattcccgaagtgtggcaccccccggacgacgtggccgcgcggctccgggccgcagggttcggggcggtgggtgccggcgctacggcggaggaaactcgtcgaatgttgcatagagcctttgatactctagcatgagccccccgtcgaagctgatgtccctcattttacaataaatgtctgcggccgacacggtcggaatctccgcgtccgtgggtttctctgcgttgcgccggaccacgagcacaaacgtgctctgccacacgtgggcgacgaaccggtaccccgggcacgcggtgagcatccggtctatgagccggtagtgcaggtgggcggacgtgccgggaaagatgacgtacagcatgtggcccccgtaagtggggtccgggtaaaacaacagccgcgggtcgcacgccccgcctccgcgcaggatcgtgtggacgaaaaaaagctcgggttggccaagaatcccggccaagaggtcctggaggggggcgttgtggcggtcggccaacacgaccaaggaggccaggaaggcgcgatgctcgaatatcgtgttgatctgctgcacgaaggccaggattagggcctcgcggctggtggcggcgaaccgcccgtctcccgcgttgcacgcgggacagcaacccccgatgcctaggtagtagcccatcccggagagggtcaggcagttgtcggccacggtctggtccagacagaagggcagcgacacgggagtggtcttcaccaggggcaccgagaacgagcgcacgatggcgatctcctcggagggcgtctgggcgagggcggcgaaaaggccccgatagcgctggcgctcgtgtaaacacagctcctgtttgcgggcgtgaggcggcaggctcttccgggaggcccgacgcaccacgcccagagtcccgccggccgcagaggagcacgaccgccggcgctccttgccgtgatagggcccgggccgggagccgcggcgatgggggtcggtatcatacataggtacacagggtgtgctccagggacaggagcgagatcgagtggcgtctaagcagcgcgcccgcctcacggacaaatgtggcgagcgcggtgggctttggtacaaatacctgatacgtcttgaaggtgtagatgagggcacgcaacgctatgcagacacgcccctcgaactcgttcccgcaggccagcttggccttgtggagcagcagctcgtcgggatgggtggcggggggatggccgaacagaacccaggggtcaacctccatctccgtgatggcgcacatggggtcacagaacatgtgcttaaagatggcctcgggccccgcggcccgcagcaggctcacaaaccggcccccgtccccgggctgcgtctcggggtccgcctcgagctggtcgacgacgggtacgatacagtcgaagaggctcgtgttgttttccgagtagcggaccacggaggcccggagtctgcgcagggccagccagtaagcccgcaccagtaacaggttacacagcaggcattctccgccggtgcgcccgcgcccccggccgtgtttcagcacggtggccatcagagggcccaggtcgaggtcgggctgggcatcgggttcggtaaactgcgcaaagcgcggagccacgtcgcgcgtgcgtgccccgcgatgcgcttcccaggactggcggaccgtggcgcgacgggcctccgcggcagcgcgcagctggggccccgactcccagacggcgggggtgccggcgaggagcagcaggaccagatccgcgtacgcccacgtatccggcgactcctccggctcgcggtccccggcgaccgtctcgaattccccgttgcgagcggcggcgcgcgtacagcagctgtccccgcccccgcgccgaccctccgtgcagtccaggagacgggcgcaatccttccagttcatcagcgcggtggtgagcgacggctgcgtgccggatcccgccgccgaccccgccccctcctcgcccccggaggccaaggttccgatgagggcccgggtggcagactgcgccaggaacgagtagttggagtactgcaccttggcggctcccggggagggcgagggcttgggttgcttctgggcatgccgcccgggcaccccgccgtcggtacggaagcagcagtggagaaaaaagtgccggtggatgtcgtttatggtgagggcaaagcgtgcgaaggagccgaccagggtcgccttcttggtgcgcagaaagtggcggtccatgacgtacacaaactcgaacgcggccacgaagatgctagcggcgcagtggggcgcccccaggcatttggcacagagaaacgcgtaatcggccacccactggggcgagaggcggtaggtttgcttgtacagctcgatggtgcggcagaccagacagggccggtccagcgcgaaggtgtcgatggccgccgcggaaaagggcccggtgtccaaaagcccctccccacagggatccgggggcgggttgcggggtcctccgcgcccgcccgaaccccctccgtcgcccgcccccccgcgggcccttgagggggcggtgaccacgtcggcggcgacgtcctcgtcgagcgtaccgacgggcggcacacctatcacgtgactggccgccaggagctcggcgcagagagcctcgttaagagccaggaggctgggatcgaaggccacatacgcgcgctcgaacgcccccgccttccagctgctgccgggggactcttcgcacaccgcgacgctcgccaggaccccggggggcgaagttgccatggctgggcgggaggggcgcacgcgccagcgaactttacgggacacaatccccgactgcgcgctgcggtcccagaccctggagagtctagacgcgcgctacgtctcgcgagacggcgcgcatgacgcggccgtctggttcgaggatatgacccccgccgagctggaggttgtcttcccgactacggacgccaagctgaactacctgtcgcggacgcagcggctggcctccctcctgacgtacgccgggcctataaaagcgcccgacgacgccgccgccccgcagaccccggacaccgcgtgtgtgcacggcgagctgctcgcccgcaagcgggaaagattcgcggcggtcattaaccggttcctggacctgcaccagattctgcggggctgacgcgcgcgctgttgggcgggacggttcgcgaaccctttggtgggtttacgcgggcacgcacgctcccatcgcgggcgccatggcgggactgggcaagccctaccccggccacccaggtgacgccttcgagggtctcgttcagcgaattcggcttatcgtcccatctacgttgcggggcggggacggggaggcgggcccctactctccctccagcctcccctccaggtgcgcctttcagtttcatggccatgacgggtccgacgagtcgtttcccatcgagtatgtactgcggcttatgaacgactgggccgaggtcccgtgcaacccttacctgcgcatacagaacaccggcgtgtcggtgctgtttcaggggttttttcatcgcccacacaacgcccccgggggcgcgattacgccagagcggaccaatgtgatcctgggctccaccgagacgacggggctgtccctcggcgacctggacaccatcaaggggcggctcggcctggatgcccggccgatgatggccagcatgtggatcagctgctttgtgcgcatgccccgcgtgcagctcgcgtttcggttcatgggccccgaagatgccggacggacgagacggatcctgtgccgcgccgccgagcaggctattacccgtcgccgccgaacccggcggtcccgggaggcgtacggggccgaggccgggctgggggtggctggaacgggtttccgggccaggggggacggttttggcccgctccccttgttaacccaagggccctcccgcccgtggcaccaggccctgcggggtcttaagcacctacggattggcccccccgcgctcgttttggcggcgggactcgtcctgggggccgctatttggtgggtggttggtgctggcgcgcgcctataaaaaaggacgcaccgccgccctaatcgccagtgcgttccggacgccttcgccccacacagccctcccgtccgacacccccatatcgcttcccgacctccggtcccgatggccgtcccgcaatttcaccgccccagcaccgttaccaccgatagcgtccgggcgcttggcatgcgcgggctcgtcttggccaccaataactctcagtttatcatggataacaaccacccgcacccccagggcacccaaggggccgtgcgggagtttctccgcggtcaggcggcggcgctgacggaccttggtctggcccacgcaaacaacacgtttaccccgcagcctatgttcgcgggcgacgccccggccgcctggttgcggcccgcgtttggcctgcggcgcacctattcaccgtttgtcgttcgagaaccttcgacgcccgggaccccgtgaggcccggggagttccttctggggtgttttaatcaataaaagaccacaccaacgcacgagccttgcgtttaatgtcgtgtttattcaagggagtgggatagggttcgacggttcgaaacttaacacacaaaataatcgagcgcgtctagcccagtaacatgcgcacgtgatgtaggctggtcagcacggcgtcgctgtgatgaagcagcgcccggcgggtccgctgtaactgctgttgtaggcggtaacaggcgcggatcagcaccgccagggcgctacgaccggtgcgttgcacgtagcgtcgcgacagaactgcgtttgccgatacgggcggggggccgaattgtaagcgcgtcacctcttgggagtcatcggcggataacgcactgaatggttcgttggttatgggggagtgtggttcccgagggagtgggtcgagcgcctcggcctcggaatccgagaggaacaacgaggtggtgtcggagtcttcgtcgtcagagacatacagggtctgaagcagcgacacgggcgggggggtagcgtcaatgtgtagcgcgagggaggatgcccacgaagacaccccagacaaggagctgcccgtgcgtggatttgtggacgacgcggaagccgggacggatgggcggttttgcggtgcccggaaccgaaccgccggatactccccgggtgctacatgcccgttttggggctggggttggggctggggctggggcggggggtggggggggttggggtggggctggggtggggctggggttggggctggggctggggctggggctggggtggggttggggttggggctggggctggggtggggtggggctggggtggggttggggctggggttggggctggggttggggctggggtggggctggggttggggttggggttggggctggggttggggctggggcgcggacaggcggttgacggtcaaatgcccccgggggcgcgcagatgtggtgggcgtggccaccggctgccgggtaggggggcggcggggaaccgggcctccgggcgtaacaccgccctccagcgtcaaggatgtggggggcgggcctgacgtcgggggcggggtgacgggttggaccgcgggaggcgggggagagggacctgcgggagaggatgaggtcggctcggccgggttgcggcctaaaacaggggccgtggggtcggcggggtcccagggtgaagggagggattcccgcgattcggacagcgacgcgacagcggggcgcgtaaggcgccgctgcggcccgcctacgggaaccctggggggggttggcgcgggacccgaggttagcggggggcggcggttttcgcccccgggcaaaaccgtgccggttgcgaccgggggcggaacgggatcgatagggagagcgggagaagcctggccggcgaactggggaccgagcgggaggggcacaccagacaccaaagcgtggagcgctggctctgggggtttgggaggggccggggggcgcgcgaaatcggtaaccggggcgaccgtgtcggggagggcaggcggccgccaaccctgggtggtcgcggaagcctgggtggcgcgcgccagggagcgtgcccggcggtgtcggcgcgcgcgcgacccggacgaagaagcggcagaagcgcgggaggaggcgggggggcggggggcggtggcatcggggggcgccggggaactttggggggacggcaagcgccggaagtcgtcgcgggggcccacgggcgccggccgcgtgctttcggccgggacgcccggtcgtgcttcgcgagccgtgactgccggcccagggggccgcggtgcacactgggacgtggggacggactgatcggcggtgggcgaaagggggtccggggcaaggaggggcgcggggccgccggagtcgtcagacgcgagctcctccaggccgtgaatccatgcccacatgcgaggggggacgggctcgccgggggtggcgtcggtgaatagcgtgggggccaggcttccgggccccaacgagccctccgtcccaacaaggtccgccgggccgggggtcgggttcgggaccgaggggctctggtcgtcgggggcgcgctggtacaccggatgccccgggaatagctcccccgacaggagggaggcgtcgaacggccgcccgaggatagctcgcgcgaggaaggggtcctcgtcggtggcgctggcggcgaggacgtcctcgccgcccgccacaaacgggagctcctcggtggcctcgctgccaacaaaccgcacgtcgggggggccgggggggtccgggttttcccacaacaccgcgaccggggtcatggagatgtccacgagcaccagacacggcgggccccgggcgaggggccgctcggcgatgagcgcggacaggcgcgggagctgtgccgccagacacgcgttttcaatcgggttcaggtcggcgtgcaggaggcggacggcccacgtctcgatgtcggacgacacggcatcgcgcaaggcggcgtccggcccgcgagcgcgtgagtcaaacagcgtgagacacagctccagctccgactcgcgggaaaaggccgtggtgttgcggagcgccacgacgacgggcgcgcccaggagcactgccgccagcaccaggtccatggccgtaacgcgcgccgcgggggtgcggtgggtggcggcggccggcacggcgacgtgctggcccgtgggccggtagagggcgttggggggagcggggggtgacgcctcgcgcccccccgaggggctcagcgtctgcccagattccagacgcgcggtcagaagggcgtcgaaactgtcatactctgtgtagtcgtccggaaacatgcaggtccaaagagcggccagagcggtgcttgggagacacatgcgcccgaggacgctcaccgccgccagcgcctgggcgggactcagctttcccagcgcggcgccgcgctcggttcccagctcggggaccgagcgccagggcgccagggggtcggtttcggacaacttgccgcggcgccagtctgccagccgcgtgccgaacatgaggccccgggtcggagggcctccggtcgaaaacactggcagcacgcggatgcgggcgtctggatgcggggtcaggcgctgcacgaatagcatggaatctgctgcgttctgaaacgcacgggggagggtgagatgcatgtactcgtgttggcggaccagatccaggcgccaaaaggtgtaaatgtgttccggggagctggccaccagcgccaccagcacgtcgttctcgttaaaggaaacgcggtgcctagtggagctgtggggcccgagcggcggtcccggggccgccgcgtcacccccccattccagctgggcccagcgacacccaaactcgcgcgtgagagtggtcgcgacgagggcgacgtagagctcggccgccgcatccatcgaggccccccatctcgcctggcggtggcgcacaaagcgtccgaagagctgaaagttggcggcctgggcgtcgctgagggccagctgaagccggttgatgacggtgatgacgtacatggccgtgacggtcgaggccgactccagggtgtccgtcggaagcggggggcgaatgcatgccgcctcgggacacatcagcagcgcgccgagcttgtcggtcacggccgggaagcagagcgcgtactgcagtggcgttccatccgggaccaaaaagctgggggcgaacggccgatccagcgtactggtggcctcgcgcagcaccaggggccccgggcctccgctcactcgcaggtacgcctcgccccggcggcgcagcatctgcgggtcggcctcttggccgggtggggcggacgcccgggcgcgggcgtctagggcgcgaagatccacgagcaggggcgcgggcgcggcggccgcgcccgcgcccgtctggcctgtggccttggcgtacgcgctatataagcccatgcggcgttggatgagctcccgcgcgccccggaactcctccaccgcccatggggccaggtccccggccaccgcgtcgaattccgccaacaggccccccagggtgtcaaagttcatctcccaggccacccttggcaccacctcgtcccgcagccgggcgctcaggtcggcgtgttgggccacgcgccccccgagctcctccacggccccggcccgctcggcgctcttggcgcccaggacgccctggtacttggcgggaaggcgctcgtagtcccgctgggctcgcagccccgacacagtgttggtggtgtcctgcagggcgcgaagctgctcgcatgccgcgcgaaatccctcgggcgatttccaggcccccccgcgaacgcggccgaagcgaccccatacctcgtcccactccgcctcggcctcctcgagagacctccgcagggcctcgacgcggcgacgggtgtcgaagagcgcctgcaggcgcgcgccctgtcgcgtcaggaggcccgggccgtcgccgctggccgcgtttagcgggtgcgtctcaaaggtacgctgggcatgttccaaccaggcgaccgcctgcacgtcgagctcgcgcgccttctccgtctggtccaacagaatttcgacctgatccgcgatctcctccgccgagcgcgcctggtccagcgtcttggccacggtcgccgggacggcgaccaccttcagcagggtcttcagattggccagaccctcggcctcgagctgggcccggcgctcgcgcgcggccagcacctcccgcagccccgccgtgacccgctcggtggcttcggcgcgctgctgtttggcgcgcaccacggcgtccttggtatcggccaggtcctgtcgggtcacgaatgcgacgtagtcggcgtacgccgtgtccttcacggggctctggtccacgcgctccagcgccgccacgcacgccaccagcgcgtcctcgctcgggcagggcagggtgacccctgcccggacaagctcggcggccgccgccgggtcgttgcgcaccgcggatatctcctccgcggcggcggccaggtccagcgccacgcttccgatcgcgcgccgcgcgtcggcccggagggcgtccaggcgatcgcggatatccacgtactcggcgtagcccttttgaaaaaacggcacgtactggcgcagggccggcacgccccccaagtcttccgacaggtgtaggacggcctcgtggtagtcgataaacccgtcgttcgcctgggcccgctccagcagcccccccgccagccgcagaagccgcgccaggggctcggtgtccacccgaaacatgtcggcgtacgtgtcggccgcggccccgaaggccgcgctccagtcgatgcggtgaatggctgcgagcggggggagcatggggtggcgctggttctcgggggtgtatgggttaaacgcaagggccgtctccagggcaagggtcaccgccttggcgttggttcccagcgcctgttcggcccgctttcggaagtcccgggggttgtagccgtgcgtgcccgccagcgcctgcaggcgacggagctcgaccacgtcaaactcggcaccgctttccacgcggtccagcacggcctccacgtcggcggcccagcgctcgtggctactgcgggcgcgctgggccgccatcttctctctcaggtcggcgatggcggcctcaagttcgtcggcgcggcgtcgcgtggcgccgatgacctttcccagctcctgcagggcgcgcccgctgggggagtggtccccggccgtcccttcggcgtgcaacaggcccccgaacctgccctcgtggcccgcgaggctttcccgcgcgccggtggtcgcgcgcgtcgcggcctggatcagggaggcatgctctccctccggttggttggcggcccggcgcacctggacgacaaggtcggctgccgccgaccctaaggtcgtgagctgggcgatggccccccgcgcgtccagggccaaccgagtcgccttgacgtatcccgcggcgctgtcggccatggccgctaggaaggccaggggggaggccgggtcgctggcggccgcgcccagggccgtcaccgcgtcgaccaggacgcggtgcgcccgcacggccgcatccaccgtcgacgcggggtctgccgtcgcgacggcggcgctgccggcgttgatggcgttcgagacggcgtgggctatgatcggggcgtgatcggcgaagaactgcaagagaaacggagtctctggggcgtcggcgaacaggttcttcagcaccaccacgaagctgggatgcaagccagacagagccgtcgccgtgtccggagtcgggtgctccagggcatctcggtactgccccagcagcccccacatgtccgcccgcagtgccgccgtaacctcagggggcgccccccgaacggcctcggggaggtccgaccagcccgccggcagggaggcccgcagggtcgccaggacggccggacaggcctttagccccacaaagtcagggagggggcgcaggaccccctggagtttgtgcaagaacttctcccgggcgtcgcgggccaccttcgcccgctcccgcgctccctcgagcattgcctccagggagcgcgcgcgctcccgcaaacgggcacgcgcatcgggggcgagctctgccgtcagcttggcggcatccatggcccgcgcctgccgcagcgcttcctcggccatgcgcgtggcctctggcgacagcccgccgtcgtcggggtagggcgacgcgccgggcgcaggaacaaaggccgcgtcgctgtccagctgctggcccagggccgcatctagggcgtcgaagcgccgcagctcggccagacccgagctgcggcgcgcctgctggtcgttaatgtcgcggatgctgcgcgccagctcgtccagcggcttgcgttctatcagcccttggttggcggcgtccgtcaggacggagagccaggccgccaggtcctcgggggcgtccagcgtctggccccgctggatcagatcccgcaacaggatggccgtggggctggtcgcgatcgggggcggggcgggaatggcggcgcgctgcgcgatgtcccgcgtgtgctggtcgaagacaggcagggactcgagcagctggaccacgggcacgacggcggccgaagccacgtgaaaccggcggtcgttgttgtcgctggcctgtagagccttggcgctgtatacggccccccggtaaaagtactccttaaccgcgccctcgatcgcccgacgggcctgggtccgcacctcctccagccgaacctgaacggcctcggggcccagggggggtgggcgcggagccccctgcggggccgccccggccggggcgggcattacgccgaggggcccggcgtgctgtgagaccgcgtcgaccccgcgagcgagggcgtcgagggcctcgcgcatctggcgatcctccgcctccaccctaatctcttcgccacgggcaaatttggccagagcctggactctatacagaagcggttctgggtgcgtcggggtggcgggggcaaaaagggtgtccgggtgggcctgcgagcgctccagaagccactcgccgaggcgtgtatacagattggccggcggggccgcgcgaagctgcagctccaggtccgcgagttccccgtaaaaggcgtccgtctcccgaatgacatccctagccacaaggatcagcttcgccagcgccaggcgaccgatcagagagttttcgtccagcacgtgctggacgaggggcagatgggcggccacgtcggccaggctcaggcgcgtggaggccagaaagtcccccacggccgttttccggggcagcatgctcagggtaaactccagcagggcggcggccgggccggccaccccggcctgggtgtgcgtccgggccccgttctcgatgagaaaggcgaggacgcgttcaaagaaaaaaataacacagagctccagcagccccggagaagccggatacggcgaccgtaaggcgctgatggtgagccgcgaacacgcggcgccctcgcgggccagggtggcggagcacgcggtgaacttaaccgccgtggcggccacgtttgggtgggcctcgaacagctgggcgaggtctgcgcccgggggctcgggtgagcggcgagtcttcagcgcctcgagggcctgtgaggacgccggaaccatgggcccgtcgtcctcgcccgcctcggcgaccggcggcccggccgggtcggggggtgccgaggcgaggacaggctccggaacggaggcggggaccgcggccccgacgggggtttgcctttgggggtgggtttcttcttggttttggcagggggggccgagcgtttcgttttctcccccgaagtcaggtcttcgacgctggaaggcggagtccaggtgggtcggcggcgcttgggaaggccggccgagtagcgtgcccggtgccgaccaaccgggacgacgcccatctccaggacccgcatgtcgtcgtcatcttcttcggccgcctctgcggcgggggtcttgggggcggagggaggcggtggtgggatcgcggagggtgggtcggcggaggggggatccgtgggtggggtaccctttagggccaccgcccatacatcgtcgggcgcccgattcgggcgcttggcctctggttttgccgacggaccggccgtcccccgggatgtctcggaggccctgtcgtcgcgacgggcccgggtcggtggcggcgactgggcggctgtgggcgggtgtggccccggcccccctcccccctcccgggggcccacgccgacgcagggctcccccaggcccgcgatctcgccccgcagggggtgcgtgatggccacgcgccgttcgctgaacgcttcgtcctgcatgtaagtctcgctggccccgtaaagatgcagagccgcggccgtcaagtccgcaggagccgcgggttccgggcccgacggcacgaaaaacaccatggctcccgcccaccgtacgtccgggcgatcgcgggtgtaatacgtcaggtatggatacatgtcccccgcccgcactttggcgatgaacgcgggggtgccctccggaaggccatgcgggtcaaaaaggtatgcggtgtcgccgtccctgaacagccccatccctagggggccaatggttaggagcgtgtacgacagggggcgcagggcccacgggccggcgaagaacgtgtgtgcggggcattgtgtctccagcaggcctgccgcgggctccccgaagaagcccacctcgccgtatacgcgcgagaagacacagcgcagtccgccgcgcgcccctgggtactcgaggaagttggggagctcgacgatcgaacacatgcgcggcggcccagggcccgcagtcgcgcgcgtccactcgcccccctcgaccaaacatccctcgatggcctccgcggacagaacgtcgcgagggcccacatcaaatatgaggctgagaaaggacagcgacgagcgcatgcacgataccgacccccccggctccaggtcgggcgcgaactggttccgagcaccggtgaccacgatgtcgcgatcccccccgcgttccatcgtggagtgcggtggggtgcccgcgatcatatgtgccctgcgggccagagacccggcctgtttatggaccggacccccggggttagtgttgtttccgccacccacgcccccgtaccatggccccggttcccctgattaggctacgagtcgcggtgatcgcttcccaaaaaccgagctgcgtttgtctgtcttggtcttccccccccccagcccgcacaccataacaccgagaacaacacacgggggtgggcggaacataataaagctttattggtaactagttaacggcaagtccgtgggtggcgcgacggtgtcctccgggatcatctcgtcgtcctcgacgggggtgttggaatgaggcgcctcctcgcggtccacctggcgtgggccgtgcccataggcctccggcttctgtgcgtccatgggcgtaggcgcggggagactgtttccggcgtcgcggacctccaggtccctgggagcctccggtccggctaacggacgaaacgcggaagcgcgaaacacgccgtcggtgacccgcaggagctcgttcatcagtaaccaatccatactcagcgtaacggccagcccctggcgagacagatccacggagtccggaaccgcggtcgtctggcccagggggccgaggctgtagtccccccaggcccctaggtcgcgacggctcgtaagcacgacgcggtcggccgcggggctttgcgggggggcgtcctcgggcgcatgcgccattacctctcggatggccgcggcgcgctggtcggccgagctgaccaagggcgccacgaccacggcgcgctccgtctgcaggcccttccacgtgtcgtggagttcctggacaaactcggccacgggctcgggtcccgcggccgcgcgcgcggcttgatagcaggccgacagacgccgccagcgcgctagaaactgacccatgaaacaaaacccggggacctggtctcccgacagcagcttcgacgcccgggcgtgaatgccggacacgacggacagaaacccgtgaatttcgcgccggaccacggccagcacgttgtcctcgtgcgacacctgggctgccagctcgtcgcacacccccaggtgcgccgtggtttcggtgatgacggaacgcaggctcgcgagggacgcgaccagcgcgcgcttggcgtcgtgatacatgctgcagtactgactcaccgcgtcccccatggcctcggggggccagggccccaggcggtcgggcgtgtccccgaccaccgcatacaggcggcgcccgtcgctctcgaaccgacactcgaaaaaggcggagagcgtgcgcatgtgcagccgcagcagcacgatggcgtcctccagttggcgaatcagggggtcggcgcgctcggcgaggtcctgcagcaccccccgggcagccagggcgtacatgctaatcaacaggaggctggtgcccacctcggggggcggggggggctgcagttggaccaggggccgcagctgctcgacggcacccctggagatcacgtacagctcccggagcagctgctctatgttgtcggccatctgcatagtggggccgaggccgccccgggcggccggttcgaggagagtgatcagcgcgcccagtttggtgcgatggccctcgaccgtggggagatagcccagcccaaagtcccgggcccaggccaacacacgcagggcgaactcgaccgggcggggaaggtaggccgcgctacacgtggccctcagcgcgtccccaaccaccagggccagaacgtaggggacgaagcccgggtcggcgaggacgttggggtgaatgccctcgagggcggggaagcggatctgggtcgccgcggccaggtggacagagggggcatggctgggctgcccgacggggagaagcgcggacagcggcgtggccggggtggtgggggtgatgtcccagtgggtctgaccatacacgtcgatccagatgagcgccgtctcgcggagaaggctgggttgaccggaactaaagcggcgctcggccgtctcaaactcccccacgagcgcccgccgcaggctcgccagatgttccgtcggcacggccggccccatgatacgcgccagcgtctggctcagaacgccccccgacaggccgaccgcctcacagagccgcccgtgcgtgtgctcgctggcgccctggacccgcctgaaagtttttacgtagttggcatagtacccgtattcccgcgccagaccaaacacgttcgaccccgcgagggcaatgcacccaaagagctgctggacttcgccgagtccgtggccggcgggcgtccgcgcggggacgcccgccgccagaaacccctccagggccgaaaggtagtgcgtgcagtgcgagggcgtgaacccagcgtcgatcagggtgttgatcaccacggagggcgaattggtattctggatcaacgtccacgtctgctgcagcagagccagcagccgctgctgggcgccggcggagggctgctccccgagctgcagcaggctggagacggcaggctggaagactgccagtgccgacgaactcaggaacggcacgtcgggatcaaacacggccacgtccgtccgcacgcgcgccattagcgtccccgggggcgcacaggccgagcgcgggctgacgcggctgagggccgtcgacacgcgcacctcctcgcggctgcgaaccatcttgttggcctccagtggcggaatcattatggccgggtcgatctcccgcacggtgtgctgaaactgcgccaacaggggcggcgggaccacagccccccgctcgggggtcgtcaggtactcgtccaccagggccaacgtaaagagggcccgtgtgaggggagtgagggtcgcgtcgtctatgcgctggaggtgcgccgagaacagcgtcacccgattactcaccagggccaagaaccggaggccctcttgcacgaacggggcggggaagagcaggctgtacaccggggtggtaaggttcgcgctgggctgccccaacgggaccggcgccagcttgagcgacgtctccccaagggcctcgatggaggtccgcgggctcatggccaagcagctcttggtgacggtttgccagcggtctatccactccacggcgcactggcggacgcggaccggccccagggccgccgcggtgcgcaggccggcggactccagcgcatgggacgtgtcggagccggtgaccgcgaggatggtgtccttgatgacctccatctcccggaaggcctggtcgggggcctcggggagagccaccaccaagcggtgtacgagcaacccggggaggttctcggccaagagcgccgtctccggaagcccgtgggcccggtggagcgcgcacaggtgttccagcagcggccgccagcatgcccgcgcgtctgccggggcgatggccgttcccgacaacagaaacgccgccatggcggcgcgcagcttggccgtggccagaaacgccgggtcgtccgccccgtttgccgtctcggccgtgggggttggcggttggcgaaggccggctaggctcgccaataggcgctgcataggtccgtccgagggcggaccggcgggtgaggtcgtgacgacgggggcctcggacgggagaccgcggtctgccatgacgcccggctcgcgtgggtgggggacagcgtagaccaacgacgagaccgggcgggaatgactgtcgtgcgctgtagggagcggcgaattatcgatcccccgcggccctccaggaaccccgcaggcgttgcgagtaccccgcgtcttcgcggggtgttatacggccacttaagtcccggcatcccgttcgcggacccaggcccgggggattgtccggatgtgcgggcagcccggacggcgtgggttgcggactttcggcggggcggcccaaatggccctttaaacgtgtgtatacggacgcgccgggccagtcggccaacacaacccaccggaggcggtagccgcgtttggctgtggggtgggtggttccgccttgcgtgagtgtcctttcgacccccccctcccccgggtcttgctaggtcgcgatctgtggtcgcaatgaagaccaatccgctacccgcaaccccttccgtgtggggcgggagtaccgtggaactcccccccaccacacgcgataccgcggggcagggcctgcttcggcgcgtcctgcgccccccgatctctcgccgcgacggcccagtgctccccagggggtcgggaccccggagggcggccagcacgctgtggttgcttggcctggacggcacagacgcgccccctggggcgctgacccccaacgacgataccgaacaggccctggacaagatcctgcggggcaccatgcgcgggggggcggccctgatcggctccccgcgccatcatctaacccgccaagtgatcctgacggatctgtgccaacccaacgcggatcgtgccgggacgctgcttctggcgctgcggcaccccgccgacctgcctcacctggcccaccagcgcgccccgccaggccggcagaccgagcggctgggcgaggcctggggccagctgatggaggcgaccgccctggggtcggggcgagccgagagcgggtgcacgcgcgcgggcctcgtgtcgtttaacttcctggtggcggcgtgtgccgcctcgtacgacgcgcgcgacgccgccgatgcggtacgggcccacgtcacggccaactaccgcgggacgcgggtgggggcgcgcctggatcgtttttccgagtgtctgcgcgccatggttcacacgcacgtcttcccccacgaggtcatgcggtttttcggggggctggtgtcgtgggtcacccaggacgagctagcgagcgtcaccgccgtgtgcgccgggccccaggaggcggcgcacaccggccacccgggccggccccgctcggccgtgatcctcccggtgtgtgcgttcgtggacctggacgccgagctggggctggggggcccgggcgcggcgtttctgtacctggtattcacttaccgccagcgccgggaccaggagctgtgttgtgtgtacgtgatcaagagccagctccccccgcgcgggttggagccggccctggagcggctgtttgggcgcctccggatcaccaacacgattcacggcaccgaggacatgacgcccccggccccaaaccgaaaccccgacttccccctcgcgggcctggccgccaatccccaaaccccgcgttgctctgctggccaggtcacgaacccccagttcgccgacaggctgtaccgctggcagccggacctgcgggggcgccccaccgcacgcacctgtacgtacgccgcctttgcagagctcggcatgatgcccgaggatagtccccgctgcctgcaccgcaccgagcgctttggggcggtcagcgtccccgttgtcatcctggaaggcgtggtgtggcgccccggcgagtggcgggcctgcgcgtgagcgtagcaaacgccccgcccacacaacgctccgcccccaaccccttccccgctgtcactcgttgttcgttgacccggacgtccgccaaataaagccactgaaacccgaaacgcgagtgttgtaacgtcctttgggcgggaggaagccacaaaatgcaaatgggatacatggaaggaacacacccccgtgactcaggacatcggcgtgtccttttgggtttcactgaaactggcccgcgccccacccctgcgcgatgtggataaaaagccagcgcgggtggtttagggtaccacaggtgggtgctttggaaacttgtcggtcgccgtgctcctgtgagcttgcgtccctccccggtttcctttgcgctcccgccttccggacctgctctcgcctatcttctttggctctcggtgcgattcgtcaggcagtggccttgtcgaatctcgaccccaccactcgccggacccgccgacgtcccctctcgagcccgccgaaacccgccgcgtctgttgaaatggccagccgccccgccgcatcctctcccgtcgaagcgcgggccccggttgggggacaggaggccggcggccccagcgcagccacccagggggaggccgccggggcccctctcgcccgcggccaccacgtgtactgccagcgagtcaatggcgtgatggtgctttccgacaagacgcccgggtccgcgtcctaccgcatcagcgatagcaactttgtccaatgtggttccaactgcaccatgatcatagacggagacgtggtgcgcgggcgcccccaggacccgggggccgcggcatcccccgctcccttcgttgcggtgacaaacatcggagccggcagcgacggcgggaccgccgtcgtggcattcgggggaaccccacgtcgctcggcggggacgtctaccggtacccagacgaccgacgtccccaccgaggcccttgggggcccccctcctcctccccgcttcaccctgggtggcggctgttgttcctgtcgcgacacacggcgccgctctgcggtattcgggggggagggggatcccgtcggccccgcggagttcgtctcggacgaccggtcgtccgattccgactcggatgactcggaggacaccgactcggagacgctgtcacacgcctcctcggacgtgtccggcggggccacgtacgacgacgcccttgactccgattcgtcatcggatgactccctgcagatagatggccccgtgtgtcgcccgtggagcaatgacaccgcgcccctggatgtttgccccgggacccccggcccgggcgccgacgccggtggtccctcagcggtagacccacacgcaccgacgccaggggccggcgctggtcttgcggccgatcccgccgtggcccgggacgacgcggaggggctttcggacccccggccacgtctgggaacgggcacggcctaccccgtccccctggaactcacgcccgagaacgcggaggccgtggcgcgctttctgggagatgccgtgaaccgcgaacccgcgctcatgctggagtacttttgccggtgcgcccgcgaggaaaccaagcgtgtcccccccaggacattctgcagcccccctcgcctcacggaggacgactttgggcttctcaactacgcgctcgtggagatgcagcgcctgtgtctggacgttcctccggtcccgccgaacgcatacatgccctattatctcagggagtatgtgacgcggctggtcaacgggttcaagccgctggtgagccggtccgctcgcctttaccgcatcctgggggttctggtgcacctgcggatccggacccgggaggcctcctttgaggagtggctgcgatccaaggaagtggccctggactttggcctgacggaaaggcttcgcgagcacgaagcccagctggtgatcctggcccaggctctggaccattacgactgtctgatccacagcacaccgcacacgctggtcgagcgggggctgcaatcggccctgaagtatgaggagttttacctaaagcgctttggcgggcactacatggagtccgtcttccagatgtacacccgcatcgccggctttttggcctgccgggccacgcgcggcatgcgccacatcgccctggggcgagaggggtcgtggtgggaaatgttcaagttcttttccaccgcctctacgaccaccagatcgtaccgtcgacccccgccatgctgaacctggggacccgcaactactacacctccagctgctacctggtaaacccccaggccaccacaaacaaggcgaccctgcgggccatcaccagcaacgtcagcgccatcctcgcccgcaacgggggcatcgggctatgcgtgcaggcgtttaacgactccggccccgggaccgctagcgtcatacccgccctcaaggtcctcgactcgctggtggcggcgcacaacaaagagagcgcgcgtccaaccggcgcgtgcgtgtacctggagccgtggcacaccgacgtgcgggccgtgctccggatgaagggggtcctcgccggcgaagaggcccagcgctgcgacaatatcttcagcgccctctggatgccagacctgtttttcaagcgcctgattcgccacctggacggcgagaagaacgtcacatggaccctgttcgaccgggacaccagcatgtcgctcgccgactttcacggggaggagttcgagaagctctaccagcacctcgaggtcatggggttcggcgagcagatacccatccaggagctggcctatggcattgtgcgcagtgcggccacgaccgggagccccttcgtcatgttcaaagacgcggtgaaccgccactacatctacgacacccagggggcggccatcgccggctccaacctctgcaccgagatcgtccatccggcctccaagcgatccagtggggtctgcaatctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggaatcggcatgcagggcctgcacacggcctgcctgaagctggggctggatctggagtctgtcgaatttcaggacctgaacaaacacatcgccgaggtgatgctgctgtcggcgatgaagaccagcaacgcgctgtgcgttcgcggggccagtcccttcaaccactttaagcgcagcatgtatcgcgccggccgctttcactgggagcgctttccggacgcccggccgcggtacgagggcgagtgggagatgctacgccagagcatgatgaaacacggcctgcgcaacagccagtttgtcgcgctgatgcccaccgccgcctcggcgcagatctcggacgtcagcgagggctttgcccccctgttcaccaacctgttcagcaaggtgacccgggacggcgagacgctgcgccccaacacgctcctgctaaaggaactggaacgcacgtttagcgggaagcgcctcctggaggtgatggacagtctcgacgccaagcagtggtccgtggcgcaggcgctcccgtgcctggagcccacccaccccctccggcgattcaagaccgcgtttgactacgaccagaagttgctgatcgacctgtgtgcggaccgcgccccctacgtcgaccatagccaatccatgaccctgtatgtcacggagaaggcggacgggaccctcccagcctccaccctggtccgccttctggtccacgcatataagcgcggactaaaaacagggatgtactactgcaaggttcgcaaggcgaccaacagcggggtctttggcggcgacgacaacattgtctgcacgagctgcgcgctgtgcccgacaaccccctccgcgccaggcccgccgccactgtcgtcgccgtcccacgcgctcccccgctgccatggattccgcggccccagccctctcccccgctctgacggcccatacgggccagagcacgccggcggacctggcgatccagattccaaagtgccccgaccccgagaggtacttctacacctcccagtgtcccgacattaaccacctgcgctccctcagcatccttaaccgctggctggaaaccgagcttgttttcgtgggggacgaggaggacgtctccaagctttccgagggcgagctcagcttttaccgcttcctcttcgctttcctgtcggccgccgacgacctggttacggaaaacctgggcggcctctccggcctgtttgagcagaaggacattctccactactacgtggagcaggaatgcatcgaagtcgtacactcgcgcgtgtacaacatcatccagctggtgctttttcacaacaacgaccaggcgcgccgcgagtacgtggccggcaccatcaaccacccggccatccgcgccaaggtggactggctggaagcgcgggtgcgggaatgcgcctccgttccggaaaagttcatcctcatgatcctcatcgagggcatcttttttgccgcctcgtttgccgccatcgcctaccttcgcaccaacaaccttctgcgggtcacctgccagtcaaacgacctcatcagccgggacgaggccgtgcacacgacggcctcgtgttacatctacaacaactacctcggcgggcacgccaagcccccgcccgaccgcgtgtacgggctgttccgccaggcggtcgagatcgagatcggatttatccgatcccaggcgccgacggacagccatatcctgagcccggcggcgctggcggccatcgaaaactacgtgcgattcagcgcggatcgcctgttgggccttatccacatgaagccactgttttccgccccaccccccgacgccagctttccgctgagcctcatgtccaccgacaaacacaccaattttttcgagtgtcgcagcacctcctacgccggggcggtcgtcaacgatctgtgagggtcgcggcgcgcttctacccgtgtttgcccataataaacctctgaaccaaactttgggtctcattgtgattcttgtcagggacgcgggggtgggagaggataaaaggcggcgcaaaaagcagtaaccaggtccgtccagattctgagggcataggataccataattttattggtgggtcgtttgttcggggacaagcgcgctcgtctgacgtttgggctactcgtcccagaatttggccaggacgtccttgtagaacgcgggtgggggggcctgggtccgcagctgctccagaaacctgtcggcgatatcaggggccgtgatatgccgggtcacaatagatcgcgccaggttttcgtcgcggatgtcctggtagataggcaggcgtttcagaagagtccacggcccccgctccttggggccgataagcgatatgacgtacttaatgtagcggtgttccaccagctcggtgatggtcatgggatcggggagccagtccagggactctggggcgtcgtggatgacgtggcgtcgccggctggccacataactgcggtgctcttccagcagctgcgcgttcgggacctggacgagctcgggcggggtgagtatctccgaggaggacgacctggggccggggtggcccccggtaacgtcccggggatccagggggaggtcctcgtcgtcttcgtatccgccggcgatctgttgggttagaatttcggtccacgagacgcgcatctcggtgccgccggcggccggcggcaaagggggcctggtttccgtggagcgcgagctggtgtgttcccggcggatggcccgccgggtctgagagcgactcgggggggtccagtgacattcgcgcagcacatcctccacggaggcgtaggtgttattgggatggaggtcggtgtggcagcggacaaagagggccaggaactgggggtagctcatcttaaagtactttagtatatcgcgacagttgatcgtgggaatgtagcaggcgctaatatccaacacaatatcacagcccatcaacaggaggtcagtgtctgtggtgtacacgtacgcgaccgtgttggtgtgatagaggttggcgcaggcatcgtccgcctccagctgacccgagttaatgtaggcgtaccccagggcccggagaacgcgaatacagaacagatgcgccagacgcagggccggcttcgagggcgcggcggacggcagcgcggctccggacccggccgtcccccgggtccccgaggccagagaggtgccgcgccggcgcatgttggaaaaggcagagctgggtctggagtcggtgatgggggaaggcggtggagaggcgtccacgtcactggcctcctcgtccgtccggcattgggccgtcgtgcgggccaggatggccttggctccaaacacaaccggctccatacaattgaccccgcgatcggtaacgaagatggggaaaagggacttttgggtaaacacctttaataagcgacagaggcagtgtagcgtaatggcctcgcggtcgtaactggggtatcggcgctgatatttgaccaccaacgtgtacatgacgttccacaggtccacggcgatgggggtgaagtacccggccggggccccaaggccctggcgcttgaccagatggtgtgtgtgggcaaacttcatcatcccgaacaaacccatgtcaggtcgattgtaactgcggatcggcctaactaaggcgtggttggtgcgacggtccgggacacccgagtctgtctctctgtgtatggtgacccagacaacaacaccgacacaagaggacaataatccgttaggggacgctctttataatttcgatggcccaactccacgcggattggtgcagcaccctgcatgcgccggtgtgggccaaacttccccccgctcattgcctcttccaaaagggtgtggcctaacgagctgggggcgtatttaatcaggctagcgcggcgggcctgccgtagtttctggctcggtgagcgacggtccggttgcttgggtcccctggctgccagcaaaaccccaccctcgcagcggcatacgccccctccgcgtcccgcacccgagaccccggcccggctgccctcaccaccgaagcccacctcgtcactgtggggtgttcccagcccgcattgggatgacggattcccctggcggtgtggcccccgcctcccccgtggaggacgcgtcggacgcgtccctcgggcagccggaggagggggcgccctgccaggtggtcctgcagggcgccgaacttaatggaatcctacaggcgtttgccccgctgcgcacgagccttctggactcgcttctggttatgggcgaccggggcatccttatccataacacgatctttggggagcaggtgttcctgcccctggaacactcgcaattcagtcggtatcgctggcgcggacccacggcggcgttcctgtctctcgtggaccagaagcgctccctcctgagcgtgtttcgcgccaaccagtacccggacctacgtcgggtggagttggcgatcacgggccaggccccgtttcgcacgctggttcagcgcatatggacgacgacgtccgacggcgaggccgttgagctagccagcgagacgctgatgaagcgcgaactgacgagctttgtggtgctggttccccagggaacccccgacgttcagttgcgcctgacgaggccgcagctcaccaaggtccttaacgcgaccggggccgatagtgccacgcccaccacgttcgagctcggggttaacggcaaattttccgtgttcaccacgagtacctgcgtcacatttgctgcccgcgaggagggcgtgtcgtccagcaccagcacccaggtccagatcctgtccaacgcgctcaccaaggcgggccaggcggccgccaacgccaagacggtgtacggggaaaatacccatcgcaccttctctgtggtcgtcgacgattgcagcatgcgggcggtgctccggcgactgcaggtcgccgggggcaccctcaagttcttcctcacgacccccgtccccagtctgtgcgtcaccgccaccggtcccaacgcggtatcggcggtatttctcctgaaaccccagaagatttgcctggactggctgggtcatagccaggggtctccttcagccgggagctcggcctcccgggcctctgggagcgagccaacagacagccaggactccgcgtcggacgcggtcagccacggcgatccggaagacctcgatggcgctgcccgggcgggagaggcgggggcctcgcacgcctgtccgatgccgtcgtcgaccacgcgggtcactcccacgaccaagcgggggcgctcggggggcgaggatgcgcgcgcggacacggccctaaagaaacctaagacggggtcgcccaccgcacccccgcccacagatccagtccccctggacacggaggacgactccgatgcggcggacgggacggcggcccgtcccgccgctccagacgcccggagcggaagccgttacgcgtgttactttcgcgacctcccgaccggagaagcaagccccggcgccttctccgccttccgggggggcccccaaaccccgtatggttttggattcccctgacggggcggggccttggcggccgcccaactctcgcaccatcccgggttaatgtaaataaacttggtattgcccaacactctcccgcgtgtcgcgtgtggttcatgtgtgtgcctggcgtcccccaccctcgggttcgtgtatttcctttccctgtccttataaaagccgtatgtggggcgctgacggaaccaccccgcgtgccatcacggccaaggcgcgggatgctccgcaacgacagccaccgggccgcgtccccggaggacggccagggacgggtcgacgacggacggccacacctcgcgtgcgtgggggccctggcgcgggggttcatgcatatctggcttcaggccgccacgctgggttttgcgggatcggtcgttatgtcgcgcgggccgtacgcgaatgccgcgtctggggcgttcgccgtcgggtgcgccgtgctgggctttatgcgcgcaccccctcccctcgcgcggcccaccgcgcggatatacgcctggctcaaactggcggccggtggagcggcccttgttctgtggagtctcggggagcccggaacgcagccgggggccccgggcccggccacccagtgcctggcgctgggcgccgcctatgcggcgctcctggtgctcgccgatgacgtctatccgctctttctcctcgccccggggcccctgttcgtcggcaccctggggatggtcgtcggcgggctgacgatcggaggcagcgcgcgctactggtggatcggtgggcccgccgcggccgccttggccgcggcggtgttggcgggcccgggggcgaccaccgccagggactgcttctccagggcgtgccccgaccaccgccgcgtctgcgtcatcgtcgcaggcgagtctgtttcccgccgccccccggaggacccagagcgacccggggaccccgggccaccgtcccccccgacaccccaacgatcccaggggccgccggccgatgaggtcgcaccggccggggtagcgcggcccgaaaacgtctgggtgcccgtggtcacctttctgggggcgggcgcgctcgccgtcaagacggtgcgagaacatgcccgggaaacgccgggcccgggcctgccgctgtggccccaggtgtttctcggaggccatgtggcggtggccctgacggagctgtgtcaggcgcttatgccctgggaccttacggacccgctgctgtttgttcacgccggactgcaggtcatcaacctcgggttggtgtttcggttttccgaggttgtcgtgtatgcggcgctagggggtgccgtgtggatttcgttggcgcaggtgctggggctccggcgtcgcctgcacaggaaggaccccggggacggggcccggttggcggcgacgcttcggggcctcttcttctccgtgtacgcgctggggtttggggtgggggcgctgctgtgccctccggggtcaacgggcgggtggtcgggcgattgatatatttttcaataaaaggcattagtcccgaagaccgccggtgtgtgatgatttcgccataacacccaaaccccggatggggcccgggtataaattccggaaggggacacgggctaccctcactaccgagggcgcttggtcgggaggccgcatcgaacgcacacccccatccggtggtccgtgtggaggtcgtttttcagtgcccggtctcgctttgccgggaacgctagccgatccctcgcgagggggaggcgtcgggcatggccccggggcgggtgggccttgccgtggtcctgtggagcctgttgtggctcggggcgggggtggccgggggctcggaaactgcctccaccgggcccacgatcaccgcgggagcggtgacgaacgcgagcgaggcccccacatcggggtcccccgggtcagccgccagcccggaagtcacccccacatcgaccccaacccccaacaatgtcacacaaaacaaaaccacccccaccgagccggccagccccccaacaacccccaagcccacctccacgcccaaaagcccccccacgtccccccgccccaacccaagaacaacaccccccccgccaagtcgggccgccccactaaaccccccgggcccgtgtggtgcgaccgccgcgacccattggcccggtacggctcgcgggtgcagatccgatgccggtttcggaattccacccgcatggagttccgcctccagatatggcgttactccatgggtccgtcccccccaatcgctccggctcccgacctagaggaggtcctgacgaacatcaccgccccacccgggggactcctggtgtacgacagcgcccccaacctgacggacccccacgtgctctgggcggagggggccggcccgggcgccgaccctccgttgtattctgtcaccgggccgctgccgacccagcggctgattatcggcgaggtgacgcccgcgacccagggaatgtattacttggcctggggccggatggacagcccgcacgagtacgggacgtgggtgcgcgtccgcatgttccgccccccgtctctgaccctccagccccacgcggtgatggagggtcagccgttcaaggcgacgtgcacggccgccgcctactacccgcgtaaccccgtggagtttgtctggttcgaggacgaccgccaggtgtttaacccgggccagatcgacacgcagacgcacgagcaccccgacgggttcaccacagtctctaccgtgacctccgaggctgtcggcggccaggtccccccgcggaccttcacctgccagatgacgtggcaccgcgactccgtgatgttctcgcgacgcaatgccaccgggctggccctggtgctgccgcggccaaccatcaccatggaatttggggtccggcatgtggtctgcacggccggctgcgtccccgagggcgtgacgtttgcctggttcctgggggacgacccctcaccggcggctaagtcggccgttacggcccaggagtcgtgcgaccaccccgggctggctacggtccggtccaccctgcccatttcgtacgactacagcgagtacatctgtcggttgaccggatatccggccgggattcccgttctagagcaccacggcagtcaccagcccccacccagggaccccaccgagcggcaggtgatcgaggcgatcgagtgggtggggattggaatcggggttctcgcggcgggggtcctggtcgtaacggcaatcgtgtacgtcgtccgcacatcacagtcgcggcagcgtcatcggcggtaacgcgagacccccccgttacctttttaatatctatatagtttggtccccctctatcccgcccaccgctgggcgctataaagccgccaccctctcttccctcaggtcatccttggtcgatcccgaacgacacacggcgtggagcaaaacgcctccccctgagccgctttcctaccaacacaacggcatgcctctgcgggcatcggaacacgcctaccggcccctgggccccgggacaccccccatgcgggctcggctccccgccgcggcctgggttggcgtcgggaccatcatcgggggagttgtgatcattgccgcgttggtcctcgtgccctcgcgggcctcgtgggcactttccccatgcgacagcggatggcacgagttcaacctcgggtgcatatcctgggatccgacccccatggagcacgagcaggcggtcggcggctgtagcgccccggcgaccctgatcccccgcgcggctgccaaacagctggccgccgtcgcacgcgtccagtcggcaagatcctcgggctactggtgggtgagcggagacggcattcgggcctgcctgcggctcgtcgacggcgtcggcggtattgaccagttttgcgaggagcccgccctcgcatatgctactatccccgcagtcccgggggctttgttcagtttgtaacttcgacccgcaacgcgctggggctgccgtgaggcgcgtgtactgcggtctgtctcgtctcctcttctccccttccctccccctccgcatcccaggatcacaccggccaacgagggttgggggggtccggcacggacccaaaataataaacacacaatcacgtgcgataaaaagaacacgcggtcccctgtggtgtttttggttatttttattaaatctcgtcgacaaacagggggaaaggggcgtggtctagcgacggcagcacgggcggaggcgttcaccggctccggcgtccttcgcgtttaagcttggtcaggagggcgctcagggcggcgacgttggtcgggccgtcgttggtcagggcgttggctcgatggcgggcgaggacgggcgaggggctcaacggcgggggcgggggccggggcggcccggggggggaaatagggcggatccccccccgtcgtacagggggttttccgcctcaatgtacggggaggccggcgctgcattcgccgtgttcacgcagacggtttcgtagacccgcatccatggtatttcctcgtagacacgccccccgtcctcgctcacggtctcgtatattgactcgtcgtcctcgtagggggcgtgccgttcgcgggccgaggcggcgtgggtggctttgcggcgggcgtcgtcgtcgtcgtcgtcggccgtcagatacgtggcttccatctggtcgggttctccctccggggcgggtccccacacccgtggccgatcgaggctccccagagacgcgcgccggacaagaagggggcacgtcgccgccggcggtcgcctgtcgggtcccgcgacgttacgggccgggaggcgcgggggcacctcccccatgtgcgtgtaatacgtggccggctgtgcggccgcagcggggggctcggcgaccgggtcgtccgcatccggaagcgggggccccgcgccgtccgcacggcgcctccggaaccgccgggtggacggcgcgggggtcgagtgtaggcgaggtcgggggaggggcgggggctcgttgtcgcgccgcgcccgctgaatcttttcccgacaggtcccaccccccgcgcgatgcccccccgggccgcgggccatgtcgtccgggggaggccccgcggaccacgtcgtccggcgagacgccacgagccgcaggatggactcgtagtggagcgacggcgccccgctgcggagcagatccgcggccagggcggccccgaaccaagccttgatgctcaactccatccgggcccagctgggggcggtcatcgtggggaacaggggggcggtggtccgacagaaacgctcctggctgtccaccgcggcccgcagatactcgttgttcaggctgtcggtggcccagacgccgtacccggtgagggtcgcgttgatgatatactgggcgtggtgatggacgatcgacagaacctccaccgtggataccacggtatccacggtcccgtacgtaccgccgctccgcttgccggtctgccacaggttggctaggcacgtcaggtggcccaggacgtcgctgaccgccgccctgagcgccatgcactgcatggagccggtcgtgccgctgggaccccggtccagatggcgcgcgaacgtttccgcgggcgcctccgggctgccgccgagcgggaggaaccggcgattggagggactcagccggtgacatacgtgcttgtccgtcgtccacagcatccaggacgcccaccggtacagcacggagacgtaggccaggagctcgttgagccgcagtgcggtgtcggtgctggggcggcttgggtccgccgggcgcataaagaacatgtactgctgaatccgatggagggcgtcgcgcaggccggccacggtggcggcgtacttggccgccgcggccccgctcttgaacggggtgcgcgccagcagctttggcgccagggtgggccgcagcagcacgtgaaggctggggtcgcagtcgcccacggggtcctcggggacgtccaggccgctgggcaccaccgtctgcaggtacttccagtactgcgtgaggatggcgcggctcaactggccgccgggcagctccacctcgcccagcgcctgggtggcggccgaagcgtagtgccggatgtactcgtagtgcgggtcgctggcgagcccgtccacgatcaaactctcgggaaccgtgttgtgttgccgcgcggccaaccggacgctgcgatcggtgcaggtcagaaacgccggctgcgcgtcgtcggagcgctgccgcaaggcgcccacggccgcgctaaggagcccctccggggtggggagcagacacccgccgaagatgcgccgctcgggaacgcccgcgttgtcgccgcggatcaggttggcaggcgtcaggcaccgcgccagccgcagggagctcgcgccgcgcgtccggcgctgcatggtgacgcccgttcggtcgggacccgccggtcggagttatgccgcgtccagggccatcggggcgctttttatcgggaggagcttatgggcgtggcgggcctcccagcccggtcgcgcgcctccccgacacgtgcgcccgcagggcggcggccccctcgtctcccatcagcagtttcctaaactgggacatgatgccacccgcggacccgcgggccaacacggacccgccgcttacgggggcgggggggaagggctccaggtccttgagcagaaaggcggggtctgccgtcccggacacgggggcccggggcgcggaggaggcggggcgcagatccacgtgctccgcggccgcgcggacgtccgcccagaacttggcgggggtggtgcgcgcgtacaggggctgggtcgctcggaggacacacgcgtagcgcaggggggtgtacgtgcccacctcgggggccgtgaatcccccgtcaaacgcggccagtgtcacgcacgccaccacggtgtcggcaaaacccagcagccgctgcaggacgagcccggcggccagaatggcgcgcgtggtcgcagcgtcgtcccggcgccggtgcgcgtccccgcacgcccgggcgtactttaaggtcactgtcgccagggccgtgtgcagcgcgtacaccgcagcgcccagcacggcgttgagcccgctgttggcgagcagccggcgcgctgcggtgtcgcccagcgcctcgtgctcggcccccacgaccgcggggcttcccaggggcagggcgcgaaacagctcctcccgcgccacgtccgcaaaggcggggtggtgcacgtgcgggtgcaggcgcgcccccacgaccaccgagagccactggaccgtctgctccgccatcaccgccaacacatccagcacgcgccccaggaaggcggcctcccgcgtcaaaacgcaccggacggcgtcgggattgaagcgggcgagcagggccccggtggccaggtacgtcatgcggccggcatagcgggcggccacgcgacagtcgcggtccagcagcgcgcgcaccccgggccagtacagcagggaccccagcgagctgcgaaacaccgcggcgtcggggccggattggggggacactaacccccccgcgctcagtaacggcacggccgcggccccgacgggacgcaacgccgtgaggctcgcgaactgccgcctcagctcggcagccctgtcgtccaggtccgacccgcgcgcctctgcgtgaaggcgcgtcccgcatacccacccgttgatggccagccgcacgacggcatccgccaaaaagctcatcgcctgggcggggctggtttttgttcgacgatccgtcaggtcaagaatcccatcgcccgtgatataccaggccaacgcctcgccctgctgcagggtttggcggaaaaacaccgcggggttgtcgggggaggcgaagtgcatgacccccacgcgcgataacccgaacgcgctatccggacacgggtaaaacccggccggatgccccagggctagggcggagcgcacggactcgtcccacacggcaacctgaggggccagtcgatccaacgggaatgccgcccggagctccgggcccggcacgcgtccctccagaacctccaccttgggcggggaacgggccccgccgccgtcctccggcccgacgtcttccgggtagtcgtcctcctcgtactgcagttcctctaggaacagcggcgacggcgccacccgcgaaccgccgacccgccccaaaatagcccgcgcgtcgacgggacccaggtatcccccctgccgggcctgcggaggaccgcggggaacctcatcatcatcgtccaggcgaccgcgcaccgactggctacgggccgcatcgggcccggggcgctgccgggacgctcggcgatgggatgagggcggggcttccgacgcgcgccgtcgtcgggctcgcgggccttcccgtcgacggcgcacgggcggctcgtcgcccgccatctcctccagagcctctagctcgctgtcgtcatccccgcggaacaccgcacgcaggtaccccatgaaccccaccccatcgcccgctggctcgtccgccacgggcgaggcgcgggggcgggtggatgcgcgcctcctgcgccccgcgggttcgcgagccgacatggtggcgatagacgcgggttatcggatgtccgctaccccccaaaaaagaaaaagaccccacagcgcggatggaggtcggggtaggtgccgccggaccccctcgcgatgggaatggacgggagcgacggggccggcgcaaaaaacgcagtatctcccgcgaaggctacccgccgccccagcccccggccaaatgcggaaacggtcccgcgctctcgcctttatacgcgggccgccctgcgacacaatcacccgtccgtggtttcgaatctacacgacaggcccgcagacgcggctaacacacacgccggcaacccagaccccagtgggttggttgcgcggtcccgtctcctggctagttctttcccccaccaccaaataatcagacgacaaccgcaggtttttgtaatgtatgtgctcgtgtttattgtggatacgaaccggggacgggaggggaaaacccagacgggggatgcgggtccggtcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcgtgttcccgcatgaccgcctcggagggcgaggtcgtgaagctggaatacgagtccaacttcgcccgaatcaacaccataaagtacccagaggcgcgggcctggttgccatgcagggtgggaggggtcgtcaacggcgcccctggctcctccgtagccgcgctgcgcaccagcgggaggttaaggtgctcgcgaatgtggtttagctcccgcagccggcgggcctcgattggcactccccggacggtgagcgctccgttgacgaacatgaagggctggaacagacccgccaactgacgccagctctccaggtcgcaacagaggcagtcaaacaggtcgggccgcatcatctgctcggcgtacgcggcccataggatctcgcgggtcaaaaatagatacaaatgcaaaaacagaacacgcgccagacgagcggtctctcggtagtacctgtccgcgatcgtggcgcgcagcatttctcccaggtcgcgatcgcgtccgcgcatgtgcgcctggcggtgcagctgccggacgctggcgcgcaggtaccggtacagggccgagcagaagttggccaacacggttcgatagctctcctcccgcgcccgtagctcggcgtggaagaaacgagagagcgcttcgtagtagagcccgaggccgtcgcgggtggccggaagcgtcgggaaggccacgtcgccgtgggcgcgaatgtcgatttgggcgcgttcggggacgtacgcgtccccccattccaccacatcgctgggcagcgttgataggaatttacactcccggtacaggtcggcgttggtcggtaacgccgaaaacaaatcctcgttccaggtatcgagcatggtacatagcgcggggcccgcgctaaagcccaagtcgtcgaggagacggttaaagagggcggcgggggggacgggcatgggcggggagggcatgagctgggcctggctcaggcgccccgttgcgtacagcggaggggccgccggggtgtttttgggacccccggccgggcgggggggtggtggcgaagcgccgtccgcgtccatgtcggcaaacagctcgtcgaccaagaggtccattgggtggggttgatacgggaaagacgatatcgggcttttgatgcgatcgtccccgcccgcccagagagtgtgggacgcccgacggcgcgggaagagaaaaacccccaaacgcgttagaggaccggacggaccttatggggggaagtgggcagcgggaaccccgtccgttcccgaggaatgacagcccgtggtcgccaccccgcatttaagcaacccgcacgggccgccccgtacctcgtgacttccccccacattggctcctgtcacgtgaaggcaaaccgagggcggctgtccaacccaccccccgccacccagtcacggtccccgtcggattgggaaacaaaggcacgcaacgccaacaccgaatgaacccctgttggtgctttattgtctgggtacggaagtttttcactcgacgggccgtctggggcgagaagcggagcgggctggggctcgaggtcgctcggtggggcgcgacgccgcagaacgccctcgagtcgccgtggccgcgtcgacgtcctgcaccacgtctggattcaccaactcgttggcgcgctgaatcaggtttttgccctcgcagaccgtcacgcggatggtggtgatgccaaggagttcgttgaggtcttcgtctgtgcgcggacgcgacatgtcccagagctggaccgccgccatccgggcatgcatggccgccaggcgcccaaccgcggcgcagaagacgcgcttgttaaagccggccacccggggggtccatggcgcgtcggggtttgggggggcggtgctaaagtgcagctttctggccagcccctgcgcgggtgtcttggatcgggttggcgccgtcgacgcgggggcgtctgggagtgcggcggattctggctgggccgatttcctgccgcgggtggtctccgccgccggggccgcgggggccttagtcgccacccgctgggttcggggggcccggggggcggtggtgggtgtgcgtccggcccctccggacccagcgggcggcggaggcgcccgcgcaggccccggggcggacaaaaccgccccggaaacgggacgccgcgtccgggggacctccgggtgttcgtcgtcttcggatgacgagcccccgtagagggcataatccgactcgtcgtactggacgaaacggacctcgccccttgggcgcgcgcgtgtctgtagggcgccacggcgggaggtgtcaggcggactatcgggactcgccatacatgaagacggggtgtagtacagatcctcgtactcatcgcgcggaacctcccgcggacccgacttcacggagcggcgagaggtcatggttccacgaacacgctagggtcggatgcgcggacaattaggcctgggttcggacggcgggggtggtgcaggtgtggagaggtcgagcgataggggcggcccgggagagaagagagggtccgcaaaacccactggggatgcgtgagtggccctctgtgggcggtgggggagagtcttataggaagtgcatataaccacaacccatgggtctaaccaatccccaggggccaagaaacagacacgccccaaacggtctcggtttccgcgaggaaggggaagtcctgggacaccctccacccccacccctcaccccacacagggcgggttcaggcgtgcccggcagccagtagcctctggcagatctgacagacgtgtgcgataatacacacgcccatcgaggccatgcctacataaaagggcaccagggcccccggggcagacatttggccagcgttttgggtctcgcaccgcgcgcccccgatcccatcgcgcccgccctcctcgccgggcggctccccgtgcgggcccgcgtctcccgccgctaaggcgacgagcaagacaaacaacaggcccgcccgacagacccttctgggggggcccatcgtccctaacaggaagatgagtcagtggggatccggggcgatccttgtccagccggacagcttgggtcgggggtacgatggcgactggcacacggccgtcgctactcgcgggggcggagtcgtgcaactgaacctggtcaacaggcgcgcggtggcttttatgccgaaggtcagcggggactccggatgggccgtcgggcgcgtctctctggacctgcgaatggctatgccggctgacttttgtgcgattattcacgcccccgcgctatccagcccagggcaccacgtaatactgggtcttatcgactcggggtaccgcggaaccgttatggccgtggtcgtagcgcctaaaaggacgcgggaatttgcccccgggaccctgcgggtcgacgtgacgttcctggacatcctggcgacccccccggccctcaccgagccgatttccctgcggcagttcccgcaactggcgcccccccctccaaccggggccgggatacgcgcagatccttggttggagggggcgctcggggacccaagcgtgactcctgccctaccggcgcgacgccgagggcggtccctcgtctatgccggcgagctgacgccggttcagacggaacacggggacggcgtacgagaagccatcgccttccttccaaaacgcgaggaggatgccggtttcgacattgtcgtccgtcgcccggtcaccgtcccggcaaacggcaccacggtcgtgcagccatccctccgcatgctccacgcggacgccgggcccgcggcctgctatgtgctggggcggtcgtcgctcaacgcccgcggcctcctggtcgttcctacgcgctggctccccgggcacgtatgtgcgtttgttgtttacaaccttacgggggttcctgtgaccctcgaggccggcgccaaggtcgcccagctcctggttgcgggggcggacgctcttccttggatccccccggacaactttcacgggaccaaagcgcttcgaaactaccccaggggtgttccggactcaaccgccgaacccaggaacccgccgctcctggtgtttacgaacgagtttgacgcggaggcccccccgagcgagcgcgggaccgggggttttggctctaccggtatttagcccatagcttggggttcgttccgggcaataaaaaacgtttgtatctcatctttcctgtgtgtagttgtttctgttggatgcctgtgggtctatcacacccgcccctccatcccacaaacacagaacacacgggttggatgaaaacacgcatttattgacccaaaacacacggagctgctcgagatgggccagggcgaggtgcggttggggaggctgtaggtctgggaacggacacgcggggacacgattccggtttggggtccgggagggcgtcgccgtttcgggcggcaggcgccagcgtaacctccgggggcggcgtgtgggggtgccccaaggagggcgcctcggtcaccccaagcccccccgagcgggttcccccggcaaccccgaaggcggagaggccaagggcccgttcggcgatggccacatcctccatgaccacgtcgctctcggccatgctccgaatagcctgggagacgagcacatccgcggacttgtcagccgcccccacggacatgtacatctgcaggatggtggccatacacgtgtccgccaggcgccgcatcttgtcctgatgggccgccacggccccgtcgatcgtgggggcctcgagcccggggtggtggcgcgccagtcgttctaggttcaccatgcaggcgtggtacgtgcgggccaaggcgcgggccttcacgaggcgtcgggtgtcgtccagggaccccagggcgtcatcgagcgtgatgggggcgggaagtagcgcgttaacgaccaccagggcctcctgcagccgcggctccgcctccgagggcggaacggccgcgcggatcatctcatattgttcctcggggcgcgctccccagccacatatagccccgagaagagaagccatcgcgggcgggtactggcccttgggcgcgcggacgcaatggggcaggaagacgggaaccgcggggagaggcgggcggccgggactcccgtggaggtgaccgcgctttatgcgaccgacgggggcgttattacctcttcgatcgccctcctcacaaactctctactgggggccgagccggtttatatattcagctacgacgcatacacgcacgatggccgtgccgacgggcccacggagcaagacaggttcgaagagagtcgggcgctctaccaagcgtcgggcgggctaaatggcgactccttccgagtaaccttttgtttattggggacggaagtgggtgggacccaccaggcccgcgggcgaacccgacccatgttcgtctgtcgcttcgagcgagcggacgacgtcgccgcgctacaggacgccctggcgcacgggaccccgctacaaccggaccacatcgccgccaccctggacgcggaggccacgttcgcgctgcatgcgaacatgatcctggctctcaccgtggccgtcaacaacgccagcccccgcaccggacgcgacgccgccgcggcgcagtatgatcagggcgcgtccctacgctcgctcgtggggcgcacgtccctgggacaacgcggccttaccacgctatacgtccaccacgaggcgcgcgtgctggccgcgtaccgcagggcgtattatggaagcgcgcagagtcccttctggtttcttagcaaattcgggcctgacgaaaaaagcctggtgctcaccactcggtactacctgcttcaggcccagcgtctggggggcgcgggggccacgtacgacctgcaggccatcaaggacatctgcgccacctacgcgattccccacgccccccgccccgacaccgtcagcgccgcgtccctgacctcgtttgccgccatcacgcggttctgttgcacgagccagtacgcccgcggggccgcggcggccgggtttccgctttacgtggagcgccgtattgcggccgacgtccgcgagaccagtgcgctggagaagttcataacccacgatcgcagttgcctgcgcgtgtccgaccgtgaattcattacgtacatttacctggcccattttgagtgtttcagccccccgcgcctagccacgcatcttcgggccgtgacgacccaggaccccaaccccgcggccaacacggagcagccctcgcccctgggcagggaggccgtggaacaatttttttgccacgtgcgcgcccaactgaatatcggggagtacgtcaaacacaacgtgaccccccgggagaccgtcctggatggcgatacggccaaggcctacctgcgcgctcgcacgtacgcgcccggggccctgacgcccgcccccgcgtattgcggggccgtggactccgccaccaaaatgatggggcgtttggcggacgccgaaaagctcctggcccccgcgggtggcccgcgttggcgcccgccagtcccggggaggatacggcgggcggcacgccgcccccacagacctgcggaatcgtcaagcgcctcctgagactggccgccacggaacaacaggacaccacgcccccggcgatcgcggcgcttatccgtaatgcggcggtgcagactcccctgcccgtctaccggatatccatggtccccacgggacaggcatttgccgcgctggcctgggacgactgggcccgcataacgcgggacgctcgcctggccgaagcggtcgtgtccgccgaagcggcggcgcaccccgaccacggcgcgctgggcaggcggctcacggatcgcatccgcgcccagggccccgtgatgccccctggcggcctggatgccggggggcagatgtacgtgaatcgcaacgagatatttaacggcgcgctggcaatcacaaacatcatcctggatctcgacatcgccctgaaggagcccgtcccctttcgccggctccacgaggccctgggccactttaggcgcggggctctggcggcggttcagctcctgtttcccgcggcccgcgtggaccccgacgcatatccctgttattttttcaaaagcgcatgtcggcccggcccggcgtccgtgggttccggcagcggactcggcaacgacgacgacggggactggtttccctgctacgacgacgccggtgatgaggagtgggcggaggacccgggcgccatggacacatcccacgatcccccggacgacgaggttgcctactttgacctgtgccacgaagtcggccccacggcggaacctcgcgaaacggattcgcccgtgtgttcctgcaccgacaagatcggactgcgggtgtgcatgcccgtccccgccccgtacgtcgtccacggttctctaacgatgcggggggtggcacgggtcatccagcaggcggtgctgttggaccgagattttgtggaggccatcgggagctacgtaaaaaacttcctgttgatcgatacgggggcccgggcgccatggacacatcccacgatcccccggacgacgaggttgcctactttgacctgtgccacgaagtcggccccacggcggaacctcgcgaaacggattcgcccgtgtgttcctgcaccgacaagatcggactgcgggtgtgcatgcccgtccccgccccgtacgtcgtccacggttctctaacgatgcggggggtggcacgggtcatccagcaggcggtgctgttggaccgagattttgtggaggccatcgggagctacgtaaaaaacttcctgttgatcgatacgggggtgtacgcccacggccacagcctgcgcttgccgtattttgccaaaatcgcccccgacgggcctgcgtgcggaaggctgctgccagtgtttgtgatcccccccgcctgcaaagacgttccggcgtttgtcgccgcgcacgccgacccgcggcgcttccattttcacgccccgcccacctatctcgcttccccccgggagatccgtgtcctgcacagcctgggtggggactatgtgagcttctttgaaaggaaggcgtcccgcaacgcgctggaacactttgggcgacgcgagaccctgacggaggtcctgggtcggtacaacgtacagccggatgcgggggggaccgtcgaggggttcgcatcggaactgctggggcggatagtcgcgtgcatcgaaacccactttcccgaacacgccggcgaatatcaggccgtatccgtccggcgggccgtcagtaaggacgactgggtcctcctacagctagtccccgttcgcggtaccctgcagcaaagcctgtcgtgtctgcgctttaagcacggccgggcgagtcgcgccacggcgcggacattcgtcgcgctgagcgtcggggccaacaaccgcctgtgcgtgtccttgtgtcagcagtgctttgccgccaaatgcgacagcaaccgcctgcacacgctgtttaccattgacgccggcacgccatgctcgccgtccgttccctgcagcacctctcaaccgtcgtcttgataacggcgtacggcctcgtgctcgtgtggtacaccgtcttcggtgccagtccgctgcaccgatgtatttacacggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacccgacgccacgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggtgtcgtgagtcccgcccacaagatggtggccccggccacctacctcttgaactacgcaggccgcatcgtatcgagcgtgttcctgcagtccccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgttgttccatcatcctgtcgggcatcgcaatgcgattgtgttatatcgccgtggtggccggggtggtgctcgtggcgcttcactacgagcaggagatccagaggcgcctgtttgatgtatgacgtcacatccaggccggcggaaaccggaacggcatatgcaaactggaaactgtcctgtcttggggcccacccacccgacgcgtcatatgtaaatgaaaatcgttcccccgaggccatgtgtagcctggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccggtggtggtgtgcagccgtgttccaaccacggtcacgcttcggtgcctctccccgattcgggcccggtcgcttgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttaggtacaataaaaacaaaacatttcaaacaaatcgccccacgtgttgtccttctttgctcatggccggcggggcgtgggtcacggcagatggcgggggtgggcccggcgtacggcctgggtgggcggagggaactaacccaacgtataaatccgtccccgctccaaggccggtgtcatagtgcccttaggagcttcccgcccgggcgcatccccccttttgcactatgacagcgacccccctcaccaacctgttcttacgggccccggacataacccacgtggcccccccttactgcctcaacgccacctggcaggccgaaacggccatgcacaccagcaaaacggactccgcttgcgtggccgtgcggagttacctggtccgcgcctcctgtgagaccagcggcacaatccactgctttttctttgcggtatacaaggacacccaccatacccctccgctgattaccgagctccgcaactttgcggacctggttaaccacccgccggtcctacgcgaactggaggataagcgcggggtgcggctgcggtgtgcgcggccgtttagcgtcgggacgattaaggacgtctctgggtccggcgcgtcctcggcgggagagtacacgataaacgggatcgtgtaccactgccactgtcggtatccgttctcaaaaacatgctggatgggggcctccgcggccctacagcacctgcgctccatcagctccagcggcatggccgcccgcgcggcagagcatcgacgcgtcaagattaaaattaaggcgtgatttccaaccccccatgaatgtgtgtaaccccccccccaaaaaaataaagagccgtaacccaaccaaaccaggcgtggtgtgagtttgtggacccaaagccctcagagacaatgcgacaggccagtatggaccgtgatacttttatttattaactcacaggggcgcttaccgccacaggaataccagaataatgaccaccacaatcgcgaccaccccaaatacagcatggcgccacaccacgccacaacagccctgtcgccggtatggggcatgatcagacgagccgcgcgccgcgcgttgggccctgtacagctcgcgcgaattgaccctaggaggccgccacgcgcccgagttttgcgttcgtcgctggtcgtcgggcgccaaagccccggacggctgttcggtcgaacgaacggccacgacagtggcataggttggggggtggtccgacatagcctcggcgtacgtcgggaggcccgacaagaggtcccttgtgatgtcgggtggggccacaagcctggtttccggaagaaacaggggggttgccaataacccgccagggccaaaactccggcgctgcgcacgtcgttcggcgcggcgccgggcgcgccgagcggctcgctgggcggcttggcgtgagcggccccgctccgacgcctcgccctctccggaggaggttggcggaattggcacggacgacaggggcccagcagagtacggtggaggtgggtccgtgggggtgtccagatcaataacgacaaacggcccctcgttcctaccagacaagctatcgtaggggggcgggggatcagcaaacgcgttccccgcgctccatagacccgcgtcgggttgcgccgcctccgaagccatggatgcgccccaaagccacgactcccgcgcgctaggtccttggggtaagggaaaaggccctactccccatccaagccagccaagttaacgggctacgccttcggggatgggactggcaccccggcggattttgttgggctggtacgcgtcgcccaaccgagggccgcgtccacgggacgcgccttttataaccccggggtcattcccaacgatcacatgcaatctaactggctcccctctcccctctccccccctctccccagcaggagcggggtgttgcgccgggggacgtctggaggagcgggaggtgcgcgggacggtggatgaggaacaggagttgttgcgcggtgagttgtcgctgtgagttgtgttgttgggcaggtgtggtggatgacgtgacgtgtgacgtgcggattgcgccgtgctttgttggtgttgttttacctgtggcagcccgggccccccgcgggcgcgcgcgcgcgcaaaaaaggcgggcggcggcccccgctcctcccccgcccctcccccgctcctcccccgctcccccccgcccccggccccgcccccaccccccccgcgcgcgcacgccgcccggaccgccgcccgccctttttgcgcccgcgcgcgcccgcggggggccctggctgccacaggtaaaacaacaccaacaaagcacggcgcaatccgcacgtcacacgtcacgtcatccaccaccctgcccaacaacacaactcacagcgacaactcccgcgcaacaactcctgttcctcatccacacgtcaccgcgcccctcccgctcctccagacgtaccccggcgcaacacccgctcctgctacaccccaccgcccctccccagccccagccctccccagccccagccctcccccgggagggggcgaggggcgggagggggcgaggggcgggagggggcgaggggcggtggtggggcgcgggcgcccccggagggttggatctctgacctgagattggcggcactgaggagagatgcccgaacccccccgagggagcgcgggacgcggtggggagggctggggctggggagggcggggcgggggggcggggcgggggggggggggggggggggcgggggccccccccccccccccccccccccccccccccccccccccccccgcccccccccgccccccgcccccccccgcccctcgccccctcccgcccctcgccccctcccgcccctcgaataaacaacgctactgcaaaacttaatcaggtcgttgccgtttattgcgtcttcgggtttcacaagcgccccgccccgtcccggcccgttacagcaccccgtccccctcgaacgcgccgccgcgtcttcgtcccaggcgccttcccagtccacaacgtcccgtcgcgggggcgtggccaagcccgcctccgcccccagcacctccacggcccccgccgccgccagcacggtgccgctgcggcccgtggccgaggcccagcgaatcccgggcggcgccggcggcagggcccccgggccgtcgtcgtcgtcgccgcgcagcaccagcgggggggcgtcgtcgtcgggctccagcagggcgcgggcgcaaaagtccctccgcggcccgcgccaccgggccgggccggcgcgcaccgcctcgcgccccagcgccacgtacacgggccgcagcggcgcgcccaggccccagcgcgcgcaggcgcggtgcgagtgggcctcctcctcgcagaagtccggcgcgccgggcgccatggcgtcggtggtccccgaggccgccgcccggccgtccagcgccggcagcacggcccggcggtactcgcgcggggacatgggcaccggcgtgtccgggccgagcgcgtgcgcacgcggtagcgccgttgccgccgcggcacggcgcagcggcggcgcgtcggggtacaggcgcgcgtgcgcggcctccacgcgcgcgaagacccccgggccgaacacgcggcccgaggccagcaccgtgcggcgcaggtcccgcgccgccggccagcgcacggcgcactgcacggcgggcagcaggtcgcacgccaggtaggcgtgctgccgcgacaccgcgggcccgtcggcgggccagtcgcaggcgcgcacggtgttgaccacgatgagccgccggtcgccggcgctggcgagcagccccagaaactccacggccccggcgaaggccaggtcccgcgtggacagcagcagcacgccctgcgcgcccagcgccgacacgtcgggggcgccggtccagttgcccgcccaggcggccgtgtccggcccgcacagccggttggccagggccgccagcaggcaggacagcccgccgcgctcggcggaccactccggcggcccccccgaggccccgccgccggccaggtcctcgcccggcagcggcgagtacagcaccaccacgcgcacgtcctcggggtcggggatctggcgcatccaggccgccaggcggcgcagcgggcccgaggcgcgcggggggccaaagaggcggcccccggcggccccgtgggggtgggggcccccacccccacggggccgccgggggccgcctctttggccccctgcgcgcctcgggcccgctgcgccgcatggcggcctggatgcgccagatccccgaccccgaggacgtgcgcgtggtggtgctgtactcgccgctgccgggcgaggacctggccggcggcggggcctcgggggggccgccggagtggtccgccgagcgcggcgggctgtcctgcctgctggcggccctggccaaccggctgtgcgggccggacacggccgcctgggcgggcaactggaccggcgcccccgacgtgtcggcgctgggcgcgcagggcgtgctgctgctgtccacgcgggacctggccttcgccggggccgtggagtttctggggctgctcgccagcgccggcgaccggcggctcatcgtggcaacaccgtgcgcgcctgcgactggcccgccgacgggcccgcggtgtcgcggcagcacgcctacctggcgtgcgacctgctgcccgccgtgcagtgcgccgtgcgctggccggcggcgcgggacctgcgccgcacggtgctggcctcgggccgcgtgttcggcccgggggtcttcgcgcgcgtggaggccgcgcacgcgcgcctgtaccccgacgcgccgccgctgcgcctgtgccgcggcggcaacgtgcgctaccgcgtgcgcacgcgcttcggcccggacacgccggtgcccatgtccccgcgcgagtaccgccgggccgtgctgccggcgctggacggccgggcggcggcctcggggaccaccgacgccatggcgcccggcgcgccggacttctgcgaggaggaggcccactcgcaccgcgcctgcgcgcgctggggcctgggcgcgccgctgcggcccgtgtacgtggcgctggggcgcgaggcggtgcgcgccggcccggcccggtggcgcgggccgcggagggacttttgcgcccgcgccctgctggagcccgacgacgacgcccccccgctggtgctgcgcggcgacgacgacgacggcccgggggccctgccgccggcgccgcccgggattcgctgggcctcggccacgggccgcagcggcaccgtgctggcggcggcgggggccgtggaggtgctgggggcggaggcgggctggccacgcccccgcgcgggacgttgtggactgggaaggcgcctgggcgaagacgcggcggcgcgtcgagggggacggggtgctgtacgggccgggacggggcggggcgcttgtgaaacccgaagacgcaataaacggcaacgacctgattaagttttgcagtagcgttgtttattcgaggggcgggagggggcgaggggcggccggcccgcacagccggttggccagggccgccagcaggcaggacagcccgccgcgctcggcggaccactccggcggcccccccgaggccccgccgccggccaggtcctcgcccggcagcggcgagtacagcaccaccacgcgcacgtcctcggggtcggggatctggcgcatccaggccgccatgcggcgcagcgggcccgaggcgcgcagggggccaaagaggcggcccccggcggccccgtgggggtgggggttctcgtcgtcgtcgccgccgcacgcggcctgggcggcgggggcgggcccggcgcaccgcgcggcgatcgaggccagggcccgcgggtcaaacatgagggccggtcgccaggggacggggaacagcgggtggtccgtgagctcggccacggcgcgcggggagcagtaggcctccagggcggcggccgcgggcgccgccgtgtggctgggcccccggggctgccgccgccagccgcccagggggtcggggccctcggcgggccggcgcgacagcgccagggggcgcgggcgggcctgcgccgcggcgcccgggccgccgcgggctgggcgggggtgggctcgggcccgggggcgtggaggggggcgcggggaggggggcgcgggcgtccgagccgggggcgtccgcgccgctcttcttcgtcttcgggggtcgcgggccgccgcctccgggcggccgggccgggccgggactcttgcgcttgcgcccctcccgcggcgcggcggaggcggcggcggccgccagcgcgtcggcggcgtccggtgcgctggccgccgccgccagcagggggcggaggctctggttctcaaacagcaggtccgcggcggcggcggccgcggagctcggcaggcgcgggtcccgcggcagcgcggggcccagggccccggcgaccaggctcacggcgcgcacggcggccacggcggcctcgctgccgccggccacgcgcaggtccccgcgcaggcgcatgagcaccagcgcgtcgcgcacgaaccgcagctcgcgcagccacgcgcgcaggcggggcgcgtcggcgtgcggcggcggcggggaagcggggcccgcgggtccctctggccgcggggggctggcgggccgggccccggccagccccgggacggccgccaggtcgccgtcgaagccctcggccagcgcctccaggatcccgcggcaggcggccaggcactcgacggccacgcggccggcctgggcgcggcgcccggcgtcggcgtcggcgtggcgggcggcgtcggggtcgtcgccccccacgggggaggcgggcgcggcggacagccgccccagggcggcgaggatccccgcggcgccgtacccggcgggcaccgcgcgctcgcccggtgcggcggcggcgacggcggcgaccccctcgtcatctgcgccggcgccggggctccccgcggcccccgtcagcgccgcgttctcgcgcgccaacaggggcgcgtaggcgcggcgcaggctggtcagcaggaagcccttctgcgcgcggtcgtatcggcggctcatggccacggcggccgccgcgtgcgccaggccccgccgaagcggccggccgccatggcgtagcccaggtggggcacggcccgcgccacgctgccggtgatgaaggagctgctgttgcgcgcggcgcccgagatccggaagcaggcctggtccagcgccacgtccccggggaccacgcgcgggttctggagccaccccatggcctccgcgtccgggggcctgcgcggggacctgcgcgtggccggcggcagcgggccgccgtggccgccggcgcgccgtggccggtcgccggggccctgggccccgcgctgccgcgggacccgcgcctgccgagctccgcggccgccgccgccgcggacctgctgtttgagaaccagagcctccgccccctgctggcggcggcggccagcgcaccggacgccgccgacgcgctggcggccgccgccgcctccgccgcgccgcgggaggggcgcaagcgcaagagtcccggcccggcccggccgcccggaggcggcggcccgcgacccccgaagacgaagaagagcggcgcggacgcccccggctcggcgcccgcgcccccctccccgcgcccccctccacgcccccgggcccagcccacccccccccacccgcggcggcccggggcgccgcggcgcaggcccgcccccgcccctggcgctgtcgcgccggccccccgagggccccgacccccgggcggctggcggcggcagccccgggggcccagccacacggcggcgcccgcggccgccgccctggaggcctactgctccccgcgcgccgtggccgagctcacggaccacccgctgttccccgtcccctggcgaccggccctcatgtttgacccgcgggccctggcctcgatcgccgcgcggtgcgccgggcccgcccccgccgcccaggccgcgtgcggcggcgacgacgacgagaacccccgggggggggggggggggggggggggggggtgggggggggggggggggggggggggggggggtgggtggggagtggcaaggaagaaacaagcccgaccaccagacagaaaatgtaaccatacccaaaccgactctgggggctgtttgtggggtcggaaccataggatgaacaaaccaccccgtacctcccgcacccaagggtgcgggtggctcatcggcatctgtccggtatgggttgttccccacccactcgcgttcggacgtcttagaatcatggcggttttctatgccgacatcggtttttcccccgcaataaacacgatgcgataaaaactgtttgtaaaatttattaaggggacaaaatgccctagcacaggggtggggttagggccgggtccccacacccaaacgcaccaaacagatgcaggcagtgggtcgagtacagccccgcgtacgaacacgtcgatgcgtgtgtcagacagcaccagaaagcacaggccatcaacaggtcgtgcatgtgtcggtgggtttggacgcggggggccatggtggtgataaagttaatggccgccgtccgccagggccacaggggcgacgtctcttggttggcccggagccactgggtgtggaccagccgcgcgtggcggcccaacatggcccctgtagccgggggcgggggatcgcgcacgtttgcagcgcacatgcgagacacctcgaccacggttcgaaagaaggcccggtggtccgcgggcaacatcaccaggtgcgcaagcgcccgggcgtccagagggtagagccctgagtcatccgaggttggctcatcgcccgggtcttgccgcaagtgcgtgtgggttgggcttccggtgggcgggacgcgaaccgcggtgtggatcccgacgcgggcccgagcgtatgctccatcttgtggggagaaggggtctgggctcgccaggggggcatacttgcccgggctatacagacccgcgagccgtacgtggttcgcggggggtgcgtggggtccggggctccctgggagaccggggttgtcgtggatccctggggtcacgcggtaccctggggtctctgggagctcgcggtactctgggttccctaggttctcggggtggtcgcggaacccggggctcccggggaacacgcggtgtcctggggattgttggcggtcggacggcttcagatggcttcgagatcgtagtgtccgcaccgactcgtagtagacccgaatctccacattgccccgccgcttgatcattatcaccccgttgcgggggtccggagatcatgcgcgggtgtcctcgaggtgcgtgaacacctctggggtgcatgccggcggacggcacgccttttaagtaaacatctgggtcgcccggcccaactggggccgggggttgggtctggctcatctcgagagacacgggggggaaccaccctccgcccagagactcgggtgatggtcgtacccgggactcaacgggttaccggattacggggactgtcggtcacggtcccgccggttcttcgatgtgccacacccaaggatgcgttgggggcgatttcgggcagcagcccgggagagcgcagcaggggacgctccgggtcgtgcacggcggttctggccgcctcccggtcctcacgcccccttttattgatctcatcgcgtacgtcggcgtacgtcctgggcccaacccgcatgttgtccaggaaggtgtccgccatttccagggcccacgacatgcttttcccgacgagcaggaagcggtccacgcaacggtcgccgccggtcgcctcgacgagggcgttcctcctgcgggaaggcacgaacgcgggtgagcccccgcgtcccccctcctccgcccccgcgtcccccctcctccgcccccgcgtcccccctcctccgcccccgcgtcccccctcctccgcccccgcgtcccccctcctccgcccccgcgtcccccctcctccgcccacccaaggtgcttacccgtgcaaaaaaggcggaccggtgggtttctgtcgtcggaggcccccggggtgcgtcccctgtgtttcgtgggtggggtgggcgggtctttcccccccgcgtccgcgtgtccctttccgatgcgatcccgatcccgagccggggcgtcgcgatgccgacgccgtccgctccgacggccctctgcgactcccgctcccggtccgcgtgctccgcagccgctcccgtcgttcgtggccggcgccgtctgcgggcgtcggtcgcgccgggcctttatgtgcgccggagagacccgccccccgccgcccgggtccgcccccggggccggcgcggagtcgggcacggcgccagtgctcgcacttcgccctaataatatatatatattgggacgaagtgcgaacgcttcgcgttctcacttcttttacccggcggccccgcccccttggggcggtcccgcccgccggccaatgggggggcggcaaggcgggcggcccaagggccgcccgccgtcccgtggtcccggcgtccggcgggcgggaccgggggcccggggacggccaacgggcgcgcggggctcgtatctcattaccgccgaaccgggaagtcggggcccgggccccgccccctgcccgttcctcgttagcatgcggaacggaagcggaaaccgccggatcgggcggtaatgagatgccatgcggggcggggcgcggacccacccgccctcgcgccccgtccatggcagatggcgcggatgggcggggccgggggttcgaccaacgggccgcggccacgggcccccggcgtgccggcgtcggggcggggtcgtgcataatggaattccgttcggggtgggcccgccggggggcggggggccggcggcctccgctgctcctccttcccgccggcccctgggactatatgagcccgaggacgccccgatcgtccacacggagcgcggctgccgacacggatccacgacccgacgcgggaccgccagagacagaccgtcagacgctcgccgcgccgggacgccgatacgcggacgaagcgcgggagggggatcggccgtccctgtcctttttccccacccaagcatcgaccggtccgcgctagttccgcgtcgacggcgggggtcgtcggggtccgtgggtctcgccccctccccccatcgagagtccgtaggtgacctaccgtgctacgtccgccgtcgcagccgtatccccggaggatcgccccgcatcggcgatggcgtcggagaacaagcagcgccccggctccccgggccccaccgacgggccgccgcccaccccgagcccagaccgcgacgagcggggggccctcgggtggggcgcggagacggaggagggcggggacgaccccgaccacgaccccgaccacccccacgacctcgacgacgcccggcgggacgggagggcccccgcggcgggcaccgacgccggcgaggacgccggggacgccgtctcgccgcgacagctggctctgctggcctccatggtagaggaggccgtccggacgatcccgacgcccgaccccgcggcctcgccgccccggacccccgcctttctagccgacgacgatgacggggacgagtacgacgacgcagccgacgccgccggcgaccgggccccggcccggggccgcgaacgggaggccccgctacgcggcgcgtatccggaccccacggaccgccgtcgccgcgcccgccggcccagccgccgcggagacgtcgtcacggccggcggcggccatcggcgtcatcgacctcgtcggactccgggtcctcgtcctcgtcgtccgcatcctcttcgtcctcgtcgtccgacgaggacgaggacgacgacggcaacgacgcggccgaccacgcacgcgaggcgcgggccgtcgggcggggtccgtcgagcgcggcgccggaagcccccgggcggacgccgcccccgcccgggccaccccccctctccgaggccgcgcccaagccccgggcggcggcgaggacccccgcggcctccgcgggccgcatcgagcgccgccgggcccgcgcggcggtggccggccgcgacgccacgggccgcttcacggccgggcagccccggcgggtcgagctggcgccgacgcggcctccggcgccttctcgcgcgctacgcgacgggtcgtcgcggggagccgtggcccggcgccgggcccccgcccccggggcgggtgctgtacggcggcctgggcgacagccgcccgggcctctggggggcgcccgaggcggaggaggcgcgacgccggttcgaggcctcgggcgccccggcggccgtgtgggcgcccgagctgggcgacgccgcgcagcagtacgccctgatcacgcggctgctgtacaccccggacgcggaggccatggggtggctccagaacccgcgcgtggtccccggggacgtggcgctggaccaggcctgcttccggatctcgggcgccgcgcgcaacagcagctccttcatcaccggcagcgtggcgcgggccgtgccccacctgggctacgccatggcggccggccgcttcggctggggcctggcgcacgcggcggccgccgtggccatgagccgccgatacgaccgcgcgcagaagggcttcctgctgaccagcctgcgccgcgcctacgcgcccctgttggcgcgcgagaacgcggcgctgacgggggccgcggggagccccggcgccggcgcagatgacgagggggtcgccgccgtcgccgccgccgcaccgggcgagcgcgcggtgcccgccgggtacggcgccgcggggatcctcgccgccctggggcggctgtccgccgcgcccgcctcccccgtggggggcgacggggcgtacagcagccgcgtgatcagggcgtactgctgcgcggcgtcgcccgctcgggcgcccacacggccgccggggcgcccgaggcctcgacccggcgtcgcgcctcctccgcctcgggcgccccccagaggcccgggcggctgtcgcccaggccgccgtacagcacccgccccgggggcgggggcccggcgccgggccacggctccccgctgacgacccgtcgcgatagcgcgcgtagaaggcgccggaggccgcgtcggcgtccagctcgacccgccggggctgcccggccgtgaagcggcccgtggcgtcgcggccggccaccgccgcgcgggcccggcggcgctcgatgcggcccgcggaggccgcgggggtcctcgccgccgcccggggcttgggcgcggcctcggagagggggggtggcccgggcgggggcggcgtccgcccgggggcttccggcgccgcgctcgacggaccccgcccgacggcccgcgcctcgcgtgcgtggtcggccgcgtcgttgccgtcgtcgtcctcgtcctcgtcggacgacgaggacgaagaggatgcggacgacgaggacgaggacccggagtccgacgaggtcgatgacgccgatggccgccgccggccgtgacgacgtctccgcggcggctgggccggcgggcgcggcgacaggcggtccgtgggggccggatacgcgccgcgccgcccgggggcttccggcgccgcgctcgacggaccccgcccgacggcccgcgcctcgcgtgcgtggtcggccgcgtcgttgccgtcgtcgtcctcgtcctcgtcggacgacgaggacgaagaggatgcggacgacgaggacgaggacccggagtccgacgaggtcgatgacgccgatggccgccgccggccgtgacgacgtctccgcggcggctgggccggcgggcgcggcgacaggcggtccgtggggtccggatacgcgccgcgtagcggggcctcccgttcgcggccccgggccggggcccggtcgccggcggcgtcggctgcgtcgtcgtactcgtccccgtcatcgtcgtcggctagaaaggcgggggtccggggcggcgaggccgcggggtcgggcgtcgggatcgtccggacggcctcctctaccatggaggccagcagagccagctgtcgcggcgagacggcgtccccggcgtcctcgccggcgtcggtgcccgccgcgggggccctcccgtcccgccgggcgtcgtcgaggtcgtgggggtggtcggggtcgtggtcggggtcgtccccgccctcctccgtctccgcgccccacccgagggccccccgctcgtcgcggtctgggctcggggtgggcggcggcccgtcggtggggcccggggagccggggcgctgcttgttctccgacgccatcgccgatgcggggcgatcctcccccgacgacccccgccgtcgacgcggaactagcgcggaccggtcgatgcttgggtggggaaaaaggacagggacggccgatccccctcccgcgcttcgtccgcgtatcggcgtcccggcgcggcgagcgtctgacggtctgtctctggcggtcccgcgtcgggtcgtggatccgtgtcggcagccgcgctccgtgtggacgatcggggcgtcctcgggctcatatagtcccggggccggcgggagggaggagcagcggaggccgccggccccccgccccccggcgggcccgaccccgccccgacgccggcacgccgggggcccgtggccgcggcccgttggtcgaacccccggccccgcccatccgcgccatctgccatggacggggcgcgagggcgggtgggtccgcgccccgccccgcatggcatctcattaccgcccgatccggcggtttccgcttccgttccgcatgctaacgaggaacgggcagggggcggggcccgggccccgacttcccggttcggcggtaatgagatacgagccccgcgcgcccgttggccgtccccgggcccccgtcccgcccgccggacgccgggaccacgggacgcggagcggacggcgtcggcatcgcgacgccccggctcgggatcgggatcgcatcggaaagggacacgcggacgcgggggggaaagacccgcccaccccacccacgaaacacaggggacgcccccgggggccccgacgacagaaacccccggtccgccttttttgcacgggtaagcgccttgggtgggcggaggaggggggacgcgggggcggaggaggggggacgcgggggcggaggaggggggagcgggggggaggaggggggacgcgggggcggaggaggggggacgcgggggcggaggagggggggggggggggagccactgtggtcctccgggcgttttctggatggccgacatttccccaggcgcttttgtgccttgtgtaaaagcgcggcgtcccgctctccgatccccgcccctgggcacgcgcaagcgcaagcgccctgcccgccccctctcatcggagtctgaggtcgaatccgagacagccttggagtctgaggtcgaatccgagacagcatcggattcgaccgagtctggggaccaggaggaagccccccgcatcggtggccgtagggccccccggaggcttggggggcggttttttctggacatgtcggcggaatccaccacggggacggaaacggatgcgtcggtgtcggacgaccccgacgacacgtccgactggtcttgtgacgacattcccccacgacccaagcgggcccgggtaaacctgcggctcactagctctcccgatcggcgggatggggttatttttcctaagatggggcgggtccggtctacccgggaaacgcagccccgggcccccaccccgtcggccccaagcccaaatgcaatgctccggcgctcggtgcgccaggcccagaggcggagcagcgcacgatggacccccgacctgggctacatgcgccagtgtatcaatcagctgtttcgggtcctgcgggtcgcccgggacccccacggcagtgccaaccgcctgcgccacctgatacgcgactgttacctgatgggatactgccgagcccgtctggccccgcgcacgtggtgccgcttgctgcaggtgtccggcggaacctggggcatgcacctgcgcaacaccatacgggaggtggaggctcgattcgacgccaccgcagaacccgtgtgcaagcttccttgtttggaggccagacggtacggcccggagtgtgatcttagtaatctcgagattcatctcagcgcgacaagcgatgatgaaatctccgatgccaccgatctggaggccgccggttcggaccacacgctcgcgtcccagtccgacacggaggatgccccctcccccgttacgctggaaaccccagaaccccgcgggtccctcgctgtgcgtctggaggatgagtttggggagtttgactggaccccccaggagggctcccagccctggctgtctgcggtcgtggccgataccagctccgtggaacgcccgggcccatccgattctggggcgggtcgcgcagcagaagaccgcaagtgtctggacggctgccggaaaatgcgcttctccaccgcctgcccctatccgtgcagcgacacgtttctccggccgtgagtccggtcgccccgacccccttgtatgtccccaaaataaaagaccaaaatcaaagcgtttgtcccagcgtcttaatggcgggaagggggagagaaacagaccacgcgtacatggggggtgtttgggggtttattgacatcggggctacagggtggtaaccggatagcagatgtgaggaagtctgggccgttcgccgcgaacggcgatcagagggtccgtttcttgcggaccacggcccggtgatgtgggttgctcgtctaaaatctcgggcatacccatacacgcacaacacggacgccgcaccgaatgggacgtcgtaagggggtgggaggtagctgggtggggtttgtgcagagcaatcagggaccgcagccagcgcatacaatcgcgctcccgtccgttggtcccgggcaggaccacgccgtactggtattcgtaccggctgagcagggtctccagggggtggttgggtgccgcggggaacggggtccacgccacggtccactcgggcaaaaaccgagtcggcacggcccacggttctcccacccacgcgtctggggtcttgatggcgataaatcttaccccgagccggattttttgggcgtattcgagaaacggcacacacagatccgccgcgcctaccacccacaagtggtagaggcgaggggggctgggttggtctcggtgcaacagtcggaagcacgccacggcgtccacgacctcggtgctctccaaggggctgtcctccgcaaacaggcccgtggtggtgtttggggggcagcgacaggacctagtgcgcacgatcgggcgggtgggtttgggtaagtccatcagcggctcggccaaccgtcgaaggttggccgggcgaacgacgaccggggtacccaggggttctgatgccaaaatgcggcactgcctaagcaggaagctccacagggccgggcttgcgtcgacggaagtccggggcagggcgttgttctggtcaaggagggtcattacgttgacgacaacaacgcccatgttggtatattacaggcccgtgtccggtttggggcacttgcagatttgtaaggccacgcacggcggggagacaggccgacgcgggggctgctctaaaaatttaagggccctacggtccacagacccgccttcccgggggggcccttggagcgaccggcagcggaggcgtccgggggaggggagggttatttacgggggggtaggtcagggggtgggtcgtcaaactgccgctccttaaaaccccggggcccgtcgttcggggtgctcgttggttggcactcacggtgcggcgaatggcctgtcgtaagttttgtcgcgtttacgggggacagggcaggaggaaggaggaggccgtcccgccggagacaaagccgtcccgggtgtttcctcatggccccttttataccccagccgaggacgcgtgcctggactccccgcccccggagacccccaaaccttcccacaccacaccacccggcgatgccgagcgcctgtgtcatctgcaggagatcctggcccagatgtacggaaaccaggactaccccatagaggacgaccccagcgcggatgccgcggacgatgtcgacgaggacgccccggacgacgtggcctatccggaggaatacgcagaggagctttttctgcccggggacgcgaccggtccccttatcggggccaacgaccacatccctcccccgcgtggcgcatctccccccggtatacgacgacgcagccgggatgagattggggccacgggatttaccgcagaagagctggacgccatggacaggcaggcggctcgagccatcagccgcggcggcaagcccccctcgaccatggccaagctggtgactggcatgggctttacgatccacggagcgctcaccccaggatcggaggggtgtgtctttgacagcagccacccagattacccccaacgggtaatcgtgaaggcggggtggtacacgagcacgagccacgaggcgcgactgctgaggcgactggaccacccggcgatcctgcccctcctggacctgcatgtcgtctccggggtcacgtgtctggtcctccccaagtaccaggccgacctgtatacctatctgagtaggcgcctgaacccactgggacgcccgcagatcgcagcggtctcccggcagctcctaagcgccgttgactacattcaccgccagggcattatccaccgcgacattaagaccgaaaatatttttattaacacccccgaggacatttgcctgggggactttggtgccgcgtgcttcgtgcagggttcccgatcaagccccttcccctacggaatcgccggaaccatcgacaccaacgcccccgaggtcctggccggggatccgtataccacgaccgtcgacatttggagcgccggtctggtgatcttcgagactgccgtccacaacgcgtccttgttctcggccccccgcggccccaaaaggggcccgtgcgacagtcagatcacccgcatcatccgacaggcccaggtccacgttgacgagttttccccgcatccagaatcgcgcctcacctcgcgctaccgctcccgcgcggccgggaacaatcgcccgccttacacccgaccggcctggacccgctactacaagatggacatagacgtcgaatatctggtttgcaaagccctcaccttcgacggcgcgcttcgccccagcgccgcagagctgctttgtttgccgctgtttcaacagaaatgaccgcccccggggggcggtgctgtttgcgggttggcacaaaaagaccccgacccgcgtctgtggtgtttttggcatcatgtcgccgggcgccatgcgtgccgttgttcccattatcccattccttttggttcttgtcggtgtatcgggggttcccaccaacgtctcctccaccacccaaccccaactccagaccaccggtcgtccctcgcatgaagcccccaacatgacccagaccggcaccaccgactctcccaccgccatcagccttaccacgcccgaccacacaccccccatgccaagtatcggactggaggaggaggaggaagaggaggagggggccggggatggcgaacatcttaaggggggagatgggacccgtgacaccctaccccagtccccgggtccagccgtcccgttggccggggatgacgagaaggacaaacccaaccgtcccgtagtcccaccccccggtcccaacaactcccccgcgcgccccgagaccagtcgaccgaagacaccccccaccagtatcgggccgctggcaactcgacccacgacccaactcccctcaaaggggcgacccttggttccgacgcctcaacataccccgctgttctcgttcctcactgcctcccccgccctggacaccctcttcgtcgtcagcaccgtcatccacaccttatcgtttgtgtgtattgttgctatggcgacacacctgtgtggtggttggtccagacgcgggcgacgcacacaccctagcgtgcgttacgtgtgcctgccgcccgaacgcgggtagggtatggggcggggatggggagagcccacacgcggaaagcaagaacaataaaggcggcgggatctagttgatatgcgtctctgggtgtttttggggtgtggtgggcgcggggcggtcattggacgggggtgcagttaaatacatgcccgggacccatgaagcatgcgcgacttccgggcctcggaacccacccgaaacggccaacggacgtctgagccaggcctggctatccggagaaacagcacacgacttggcgttctgtgtgtcgcgatgtctctgcgcgcagtctggcatctggggcttttgggaagcctcgtgggggctgttcttgccgccacccatctgggacctgcggccaacacaacggaccccttaacgcacgccccagtgtcccctcaccccagccccctggggggctttgccgtccccctcgtagtcggtgggctgtgtgccgtagtcctgggggcggcgtgtctgcttgagctcctgcgtcgtacgtgccgcgggtgggggcgttaccatccctacatggacccagttgtcgtataattttttccccccccccccttctccgcatgggtgatgtcgggtccaaactcccgacaccaccagctggcatggtataaatcaccggtgcgccccccaaaccatgtccggcagggggatggggggcgaatgcggagggcacccaacaacaccgggctaaccaggaaatccgtggccccggcccccaacaaagatcgcggtagcccggccgtgtgacattatcgtccatacctaccacaccgacgaatcccctaagggggaggggccattttacgaggaggaggggtataacaaagtctgtctttaaaaagcaggggttagggagttgttcggtcataagcttcagtgcgaacgaccaactaccccgatcatcagttatccttaaggtctcttttgtgtggtgcgttccggtatggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttactagatacccccccccttaatgggtgcggggggggtcaggtctgcggggttgggatgggaccttaactccatataaagcgagtctggaaggggggaaaggcggacagtcgataagtcggtagcgggggacgcgcacctgttccgcctgtcgcacccacagctttttcgcgaaccgtcccgtttcgggatgccgtgccgcccgttgcagggcctggtgctcgtgggcctctgggtctgtgccaccagcctggttgtccgccccccccttaatgggtgcggggggggtcaggtctgcggggttgggatgggaccttaactccatataaagcgagtctggaaggggggaaaggcggacagtcgataagtcggtagcgggggacgcgcacctgttccgcctgtcgcacccacagctttttcgcgaaccgtcccgtttcgggatgccgtgccgcccgttgcagggcctggtgctcgtgggcctctgggtctgtgccaccagcctggttgtccgtggccccacggtcagtctggtatcaaactcatttgtggacgccggggccttggggcccgacggcgtagtggaggaagacctgcttattctcggggagcttcgctttgtgggggaccaggtcccccacaccacctactacgatggggtcgtagagctgtggcactaccccatgggacacaaatgcccacgggtcgtgcatgtcgtcacggtgaccgcgtgcccacgtcgccccgccgtggcatttgccctgtgtcgcgcgaccgacagcactcacagccccgcatatcccaccctggagctgaatctggcccaacagccgcttttgcgggtccggagggcgacgcgtgactatgccggggtgtacgtgttacgcgtatgggtcggggacgcaccaaacgccagcctgtttgtcctggggatggccatagccgccgaaggtactctggcgtacaacggctcggcccatggctcctgcgccccgaaactgcttccgtcttcggccccgcgtctggccccggcgagcgtataccaacccgcccctaacccggcctccaccccctcgaccaccacctccaccccctcgaccaccatccccgctccccaagcatcgaccacacccttccccacgggagacccaaaaccccaacctcacggggtcaaccacgaacccccatcgaatgccacgcgagcgacccgcgactcgcgatatgcgctaacggtgacccagataatccagatagccatccccgcgtccattatagccctggtgtttctggggagctgtatttgctttatacacagatgtcaacgccgctaccgacgctcccgccgcccgatttacagcccccagatacccacgggcatctcatgcgcggtgaacgaagcggccatggcccgcctcggagccgagctcaaatcgcatccgagcaccccccccaaatcccggcgccggtcgtcacgcacgccaatgccctccctgacggccatcgccgaagagtcggagcccgcgggggcggctgggcttccgacgccccccgtggaccccacgacatccaccccaacgcctcccctgttggtataggtccacggccactggccgggggcaccacataaccgaccgcagtcactgagttgggaataaaccggtattatttacctatatccgtgtatgtccatttctttcttccccccccccccggaaaccaaagaaggaagcaaagaatggatgggaggagttcaggaagccggggagagggcccgcggcgcatttaaggcgttgttgtgttgactttggctcttctggcgggttggtgcggtgctgtttgttgggctcccattttacccgaagatcggctgctatccccgggacatggatcgcggggcggtggtggggtttcttctcggtgtttgtgttgtatcgtgcttggcgggaacgcccaaaacgtcctggagacgggtgagtgtcggcgaggacgtttcgttgcttccagctccggggcctacggggcgcggcccgacccagaaactactatgggccgtggaacccctggatgggtgcggccccttacacccgtcgtgggtctcgctgatgccccccaagcaggtgcccgagacggtcgtggatgcggcgtgcatgcgcgctccggtcccgctggcgatggcgtacgcccccccggccccatctgcgaccgggggtctacgaacggacttcgtgtggcaggagcgcgcggccgtggttaaccggagtctggttattcacggggtccgagagacggacagcggcctgtataccctgtccgtgggcgacataaaggacccggctcgccaagtggcctcggtggtcctggtggtgcaaccggccccagttccgaccccacccccgaccccagccgattacgacgaggatgacaatgacgagggcgaggacgaaagtctcgccggcactcccgccagcgggaccccccggctcccgcctccccccgcccccccgaggtcttggcccagcgcccccgaagtctcacatgtgcgtggggtgaccgtgcgtatggagactccggaagctatcctgttttcccccggggagacgttcagcacgaacgtctccatccatgccatcgcccacgacgaccagacctactccatggacgtcgtctggttgaggttcgacgtgccgacctcgtgtgccgagatgcgaatatacgaatcgtgtctgtatcacccgcagctcccagaatgtctgtccccggccgacgcgccgtgcgccgcgagtacgtggacgtctcgcctggccgtccgcagctacgcggggtgttccagaacaaaccccccaccgcgctgttcggccgaggctcacatggagcccgtcccggggctggcgtggcaggcggcctccgtcaatctggagttccgggacgcgtccccacaacactccggcctgtatctgtgtgtggtgtacgtcaacgaccatattcacgcctggggccacattaccatcagcaccgcggcgcagtaccggaacgcggtggtggaacagcccctcccacagcgcggcgcggatttggccgagcccacccacccgcacgtcggggcccctccccacgcgcccccaacccacggcgccctgcggttaggggcggtgatgggggccgccctgctgctgtctgcgctggggttgtcggtgtgggcgtgtatgacctgttggcgcaggcgtgcctggcgggcggttaaaagcagggcctcgggtaaggggcccacgtacattcgcgtggccgacagcgagctgtacgcggactggagctcggacagcgagggagaacgcgaccaggtcccgtggctggcccccccggagagacccgactctccctccaccaatggatccggctttgagatcttatcaccaacggctccgtctgtatacccccgtagcgacgggcatcaatctcgccgccagctcacaacctttggatccggaaggcccgatcgccgttactcccaggcctccgattcgtccgtcttctggtaaggcgccccatcccgaggccccacgtcggtcgccgaactgggcgaccgccggcgaggtggacgtcggagacgagctaatcgcgatttccgacgaacgcggacccccccgacatgaccgcccgcccctcgccacgtcgaccgcgccctcgccacacccgcgacccccgggctacacggccgttgtctccccgatggccctccaggctgtcgacgccccctccctgtttgtcgcctggctggccgctcggtggctccggggggcttccggcctgggggccgtcctgtgtgggattgcgtggtatgtgacgtcaattgcccgaggcgcacaaagggccggtggtccgcctagccgcagcaaattaaaaatcgtgagtcacagcgaccgcaacttcccacccggagctttcttccggcctcgatgacgtcccggctctccgatcccaactcctcagcgcgatccgacatgtccgtgccgctttatcccacggcctcgccagtttcggtcgaagcctactactcggaaagcgaagacgaggcggccaacgacttcctcgtacgcatgggccgccaacagtcggtattaaggcgttgacgcagacgcacccgctgcgtcggcatggtgatcgcctgtctcctcgtggccgttctgtcgggcggatttggggcgctcctgatgtggctgctccgctaaaagaccgcatcgacacgcgcgtccttcttgtcgtctctcttcccccccatcaccccgcaatttgcacccagcctttaactacattaaattgggttcgattggcaatgttgtctcccggttgatttttgggtgggtggggagtgggtgggtggggagtgg

SEQ ID NO: 2 - protein, 338 aa, Herpes simplex virus 1
MLAVRSLQHLSTVVLITAYGLVLVWYTVFGASPLHRCIYAVRPTGTNNDTALVWMKMNQTLLFLGAPTHPPNGGWRNHAHICYANLIAGRVVPFQVPPDATNRRIMNVHEAVNCLETLWYTRVRLVVVGWFLYLAFVALHQRRCMFGVVSPAHKMVAPATYLLNYAGRIVSSVFLQYPYTKITRLLCELSVQRQNLVQLFETDPVTFLYHRPAIGVIVGCELMLRFVAVGLIVGTAFISRGACAITYPLFLTITTWCFVSTIGLTELYCILRRGPAPKNADKAAAPGRSKGLSGVCGRCCSIILSGIAMRLCYIAVVAGVVLVALHYEQEIQRRLFDV

SEQ ID NO: 3 - DNA, 19 bp, Herpes simplex virus 1
tccctatcagtgatagaga

SEQ ID NO: 4 - DNA, 5606 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggctgcaggtcaacaccagagcctgcccaacatggcacccccactcccacgcacccccactcccacgcacccccactcccacgcacccccactcccacgcacccccactcccacgcacccccactcccacgcacccccactcccacgcacccccactcccacgcacccccactcccacgcatccccgcgatacatccaacacagacagggaaaagatacaaaagtaaacctttatttcccaacagacagcaaaaatcccctgagtttttttttattagggccaacacaaaagacccgctggtgtgtggtgcccgtgtctttcacttttcccctccccgacacggattggctggtgtagtgggcgcggccagagaccacccagcgcccgacccccccctccccacaaacacggggggcgtcccttattgttttccctcgtcccgggtcgaccagacatgataagatacattgatgagtttggacaaaccacaactagaatgcagtgaaaaaaatgctttatttgtgaaatttgtgatgctattgctttatttgtaaccattataagctgcaataaacaagttctgctttaataagatctgaattcccgggatccgctgtacgcggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatatcaattcgccctatagtgagtcgtattacaattctttgccaaaatgatgagacagcacaataaccagcacgttgcccaggagctgtaggaaaaagaagaaggcatgaacatggttagcagaggggcccggtttggactcagagtattttatcctcatctcaaacagtgtatatcattgtaaccataaagagaaaggcaggatgatgaccaggatgtagttgtttctaccaataagaatatttccacgccagccagaatttatatgcagaaatattctaccttatcatttaattataacaattgttctctaaaactgtgctgaagtacaatataatataccctgattgccttgaaaaaaaagtgattagagaaagtacttacaatctgacaaataaacaaaagtgaatttaaaaattcgttacaaatgcaagctaaagtttaacgaaaaagttacagaaaatgaaaagaaaataagaggagacaatggttgtcaacagagtagaaagtgaaagaaacaaaattatcatgagggtccatggtgatacaagggacatcttcccattctaaacaacaccctgaaaactttgccccctccatataacatgaattttacaatagcgaaaaagaaagaacaatcaagggtccccaaactcaccctgaagttctcaggatcgatccggagctttttgcaaaagcctaggcctccaaaaaagcctcttcactacttctggaatagctcagaggccctagaggatccccggcggggtcgtatgcggctggagggtcgcggacggagggtccctgggggtcgcaacgtaggcggggcttctgtggtgatgcggagagggggcggcccgagtctgcctggctgctgcgtctcgctccgagtgccgaggtgcaaatgcgaccagactgtcgggccagggctaacttataccccacgcctttcccctccccaaaggggcggcagtgacgattcccccaatggccgcgcgtcccaggggaggcaggcccaccgcggggcggccccgtccccggggaccaacccggcgcccccaaagaatatcattagcatgcacggcccggcccccgatttgggggcccaacccggtgtcccccaaagaaccccattagcatgcccctcccgccgacgcaacaggggcttggcctgcgtcggtgccccggggcttcccgccttcccgaagaaactcattaccatacccggaaccccaggggaccaatgcgggttcattgagcgacccgcgggccaatgcgcgaggggccgtgtgttccgccaaaaaagcaattagcataacccggaaccccaggggagtggttacgcgcggcgcgggaggcggggaataccggggttgcccattaagggccgcgggaattgccggaagcgggaagggcggccggggccgcccattaatgagtttctaattaccataccgggaagcggaacaaggcctcttgcaagtttttaattaccataccgggaagtgggcggcccggcccattgggcggtaactcccgcccaatgggccgggccccgaagactcggcggacgctggttggccgggccccgccgcgctggcggccgccgattggccagtcccgcccccgaggcggcccgccctgtgagggcgggctggctccaagcgtatatatgcgcggctcctgccatcgtctctccggagagcggcttggtgcggagctcgaattcggtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 1017 bp, Herpes simplex virus 1
atgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacccgacgccacgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggtgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgttgttccatcatcctgtcgggcatcgcaatgcgattgtgttatatcgccgtggtggccggggtggtgctcgtggcgcttcactacgagcaggagatccagaggcgcctgtttgatgtatga

(19) United States
(12) Patent Application Publication
(10) Pub. No.: US 2025/0339481 A1
(43) Pub. Date: Nov. 6, 2025

(54) METHOD OF DELIVERY OF FUSOGENIC ONCOLYTIC VIRUS AND THERAPEUTIC MOLECULES

(71) Applicant: THE BRIGHAM AND WOMEN'S HOSPITAL, INC., Boston, MA (US)

(72) Inventor: Feng YAO, Southborough, MA (US)

(73) Assignee: THE BRIGHAM AND WOMEN'S HOSPITAL, INC., Boston, MA (US)

(21) Appl. No.: 18/289,204

(22) PCT Filed: May 5, 2022
(86) PCT No.: PCT/US2022/027835

§ 371 Date: Nov. 1, 2023

Related U.S. Application Data
(60) Provisional application No. 63/184,705, filed on May 5, 2021.

(51) Int. Cl. A61K 35/763; A61K 31/65; A61P 35/04; C07K 16/28

(52) U.S. Cl. CPC A61K 35/763; A61K 31/65; A61P 35/04; C07K 16/2818; C07K 16/2878

(57) ABSTRACT

Malignant tumors that are resistant to conventional therapies represent significant therapeutic challenges. An embodiment of the present invention provides a method for treating cancer comprising administering to a subject in need thereof a checkpoint inhibitor in combination with a new generation regulatable fusogenic oncolytic herpes simplex virus-1 that is more effective at selective killing target cells, such as tumor cells. In various embodiments presented herein, the methods described herein is suitable for treatment of solid tumors, as well as other cancers.

### CROSS-REFERENCE TO RELATED APPLICATIONS

This application is a 35 U.S.C. § 371 National Phase Entry Application of International Application No. PCT/US2022/027835 filed May 5, 2022, which designated the U.S., which claims benefit under 35 U.S.C. § 119 (e) of U.S. Provisional Application No. 63/184,705 filed May 5, 2021, the contents of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 20, 2022, is named 043214-097490WOPT_SL.txt and is 197,283 bytes in size.

### FIELD OF INVENTION

The present invention is directed compositions and methods of treating cancer using a checkpoint inhibitor in combination with a regulatable fusogenic oncolytic herpes simplex virus 1 (HSV-1) virus.

### BACKGROUND

Checkpoint inhibitors strengthen the immune response against a tumor by interfering with the normal inhibitory signals that regulate lymphocytes and lowering the activation signal needed to generate an immune response. However, not all tumors respond to checkpoint inhibitors as a cancer immunotherapy treatment, so it may be necessary to combine them with other possible treatment strategies. One such type of treatment strategy involves the use of oncolytic viral therapy.

Oncolytic viral therapy entails harnessing the ability of a virus to reproduce in and lyse human cells, and directing this viral replication-dependent lysis preferentially toward cancerous cells. Advances in cancer biology, together with a detailed understanding of the roles of host factors and virus-encoded gene products in controlling virus production in infected cells, have facilitated the use of some viruses as potential therapeutic agents against cancer. Herpes simplex virus (HSV) possesses several unique properties as an oncolytic agent. It can infect a broad range of cell types, leading to the replication of new virus and cell death. HSV has a short replication cycle (9 to 18 h) and encodes many non-essential genes that, when deleted, greatly restrict the ability of the virus to replicate in non-dividing normal cells. Because of its large genome, multiple therapeutic genes can be packaged into the genome of oncolytic recombinants.

The use of a replication-conditional strain of HSV-1 as an oncolytic agent was first reported for the treatment of malignant gliomas. Since then, various efforts have been made in an attempt to broaden their therapeutic efficacy and increase the replication specificity of the virus in tumor cells. Not surprisingly, however, deletion of genes that impair viral replication in normal cells also leads to a marked decrease in the oncolytic activity of the virus for the targeted tumor cells.

Currently, no oncolytic viruses that are able to kill only tumor cells while leaving normal cells intact are available. Consequently, the therapeutic doses of existing oncolytic viruses are significantly restricted. The availability of an oncolytic virus whose replication can be tightly controlled and adjusted pharmacologically would offer greatly increased safety and therapeutic efficacy. Such a regulatable oncolytic virus would minimize unwanted replication in adjacent and distant tissues as well as undesirable progeny virus overload in the target area after the tumor has been eliminated. This regulatory feature would also allow the oncolytic activity of the virus to be quickly shut down should adverse effects be detected. Work described herein presents the combination of treating with checkpoint inhibitors along with a regulatable fusogenic variant of an oncolytic HSV, which together, are significantly more effective at killing cancer cells than either treatment alone.

### SUMMARY OF THE INVENTION

Described herein is methods comprising administration of a checkpoint inhibitor and a tetracycline-regulatable HSV-1 ICP0 null mutant based fusogenic oncolytic virus, QREO5-F, whose preferential replication ability in human cancer cells over normal cells is further enhanced through series propagation of virus in human cancer cell lines. Data provided herein show that infection of multiple human cancer cell types, including breast cancer, liver cancer, melanoma, pancreatic cancer, ovarian cancer, and several different non-small cell lung cancer cells with QREO5-F lead to 36,000- to 5×10⁷-fold tetracycline-dependent progeny virus production, while little viral replication and virus-associated cytotoxicity are observed in infected growing as well as growth-arrested normal human fibroblasts. QREO5-F is, thus, a replication-competent oncolytic virus in the presence of tetracycline/doxycycline, and a replication-defective virus in the absence of tetracycline/doxycycline.

Importantly, QREO5-F is highly effective against pre-established CT26.WT colon carcinoma tumor in immune-competent mice. QREO5-F virotherapy led to induction of effective tumor-specific immunity that can prevent the tumor growth following re-challenge with the same type of tumor cells. Collectively, QREO5-F comprises efficacy and safety features suitable for clinical development.

Accordingly, one aspect described herein provides a method for treating cancer, the method comprising administering a subject in need thereof, (i) a checkpoint inhibitor; and ii) an oncolytic Herpes Simplex Virus (HSV) comprising recombinant DNA, wherein the recombinant DNA comprises: a) a gene comprising a 5′ untranslated region and a HSV-1, or HSV-2, VP5 gene that is operably linked to an VP5 promoter comprising a TATA element; b) a tetracycline operator sequence positioned between 6 and 24 nucleotides 3′ to said TATA element, wherein the VP5 gene lies 3′ to said tetracycline operator sequence; c) a gene sequence encoding tetracycline repressor operably linked to an HSV immediate-early promoter, wherein the gene sequence is located at the ICP0 locus; d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and e) a gene sequence encoding a functional ICP34.5 protein; wherein said oncolytic HSV does not encode functional ICP0 and does not contain a ribozyme sequence located in said 5′ untranslated region of VP5.

In one embodiment of any aspect described herein, the cancer is a solid tumor.

In one embodiment of any aspect described herein, the tumor is benign.

In one embodiment of any aspect described herein, the tumor is malignant.

In one embodiment of any aspect described herein, the subject is diagnosed or has been diagnosed as having cancer is selected from the list consisting of: a carcinoma, a melanoma, a sarcoma, a germ cell tumor, and a blastoma.

In one embodiment of any aspect described herein, the subject is diagnosed or has been diagnosed as having a cancer selected from the group consisting of: non-small-cell lung cancer, breast cancer, brain cancer, colon cancer, prostate cancer, liver cancer, lung cancer, ovarian cancer, skin cancer, head and neck cancer, kidney cancer, and pancreatic cancer.

In one embodiment of any aspect described herein, the cancer is metastatic.

In one embodiment of any aspect described herein, the variant gene is a gK variant gene that encodes an amino acid substitution selected from the group consisting of: an Ala to Thr amino acid substitution corresponding to amino acid 40 of SEQ ID NO: 2; an Ala to “x” amino acid substitution corresponding to amino acid 40 of SEQ ID NO: 2, wherein “x” is any amino acid; an Asp to Asn amino acid substitution corresponding to amino acid 99 of SEQ ID NO: 2; a Leu to Pro amino acid substitution corresponding to amino acid 304 of SEQ ID NO: 2; and an Arg to Leu amino acid substitution corresponding to amino acid 310 of SEQ ID NO: 2.

In one embodiment of any aspect described herein, the tetracycline operator sequence comprises two Op2 repressor binding sites.

In one embodiment of any aspects described herein, the VP5 promoter is an HSV-1 or HSV-2 VP5 promoter.

In one embodiment of any aspects described herein, the immediate-early promoter is an HSV-1 or HSV-2 immediate-early promoter.

In one embodiment of any aspects described herein, the HSV immediate-early promoter is selected from the group consisting of: ICP0 promoter, ICP27 promoter and ICP4 promoter.

In one embodiment of any aspects described herein, the recombinant DNA is part of the HSV-1 genome.

In one embodiment of any aspects described herein, the recombinant DNA is part of the HSV-2 genome.

In one embodiment of any aspects described herein, the method further comprises administering an agent that regulates the tet operator-containing promoter.

In one embodiment of any aspects described herein, the agent is doxycycline or tetracycline.

In one embodiment of any aspects described herein, the agent is administered locally or systemically.

In one embodiment of any aspects described herein, the systemic administration is oral administration.

In one embodiment of any aspects described herein, the checkpoint inhibitor and the oncolytic virus are administered directly to the tumor.

In one embodiment of any aspects described herein, the checkpoint inhibitor is administered systemically and the oncolytic virus are administered directly to the tumor.

In one embodiment of any aspects described herein, the checkpoint inhibitor and the oncolytic virus are administered in the same composition.

In one embodiment of any aspects described herein, the checkpoint inhibitor and the oncolytic virus are administered in different compositions.

In one embodiment of any aspects described herein, the checkpoint inhibitor and the oncolytic virus are administered at substantially the same time.

In one embodiment of any aspects described herein, the checkpoint inhibitor and the oncolytic virus are administered at different times.

In one embodiment of any aspects described herein, the checkpoint inhibitor is selected from the group consisting of: an anti-PD-1 antibody or antibody reagent, an anti-PD-L1 antibody or antibody reagent, an anti-OX40 antibody or antibody reagent, a CTLA-4 antibody or antibody reagent, a TIM-3 antibody or antibody reagent, and a TIGIT antibody or antibody reagent.

One aspect provided herein is a method for treating cancer, the method comprising administering a subject in need thereof, i) an anti-PD-L1 checkpoint inhibitor; and ii) an oncolytic Herpes Simplex Virus (HSV) comprising recombinant DNA, wherein the recombinant DNA comprises: a) gene comprising a 5′ untranslated region and a HSV-1, or HSV-2, VP5 gene that is operably linked to an VP5 promoter comprising a TATA element; b) tetracycline operator sequence positioned between 6 and 24 nucleotides 3′ to said TATA element, wherein the VP5 gene lies 3′ to said tetracycline operator sequence; c) a gene sequence encoding tetracycline repressor operably linked to an HSV immediate-early promoter, wherein the gene sequence is located at the ICP0 locus; d) variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and e) a gene sequence encoding a functional ICP34.5 protein; wherein said oncolytic HSV does not encode functional ICP0 and does not contain a ribozyme sequence located in said 5′ untranslated region of VP5.

One aspect provided herein describes a method for treating cancer, the method comprising administering a subject in need thereof, i) a checkpoint inhibitor; and ii) an oncolytic Herpes Simplex Virus (HSV) comprising recombinant DNA, wherein the recombinant DNA does not encode functional ICP0; and encodes fusogenic activity.

One aspect provided herein describes a kit comprising i) a checkpoint inhibitor; and ii) an oncolytic Herpes Simplex Virus (HSV) comprising recombinant DNA, wherein the recombinant DNA comprises: a) a gene comprising a 5′ untranslated region and a HSV-1, or HSV-2, VP5 gene that is operably linked to an VP5 promoter comprising a TATA element; b) a tetracycline operator sequence positioned between 6 and 24 nucleotides 3′ to said TATA element, wherein the VP5 gene lies 3′ to said tetracycline operator sequence; c) a gene sequence encoding tetracycline repressor operably linked to an HSV immediate-early promoter, wherein the gene sequence is located at the ICP0 locus; d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and e) a gene sequence encoding a functional ICP34.5 protein; wherein said oncolytic HSV does not encode functional ICP0 and does not contain a ribozyme sequence located in said 5′ untranslated region of VP5.

One aspect provided herein describes a kit comprising i) a checkpoint inhibitor; and ii) an oncolytic Herpes Simplex Virus (HSV) comprising recombinant DNA, wherein the recombinant DNA does not encode functional ICP0; and encodes fusogenic activity.

In one embodiment of any aspect described herein, the kit further comprises an agent that regulates the tet operator-containing promoter.

One aspect provided herein describes a composition comprising i) a checkpoint inhibitor; and ii) an oncolytic Herpes Simplex Virus (HSV) comprising recombinant DNA, wherein the recombinant DNA comprises: a) a gene comprising a 5′ untranslated region and a HSV-1, or HSV-2, VP5 gene that is operably linked to an VP5 promoter comprising a TATA element; b) a tetracycline operator sequence positioned between 6 and 24 nucleotides 3′ to said TATA element, wherein the VP5 gene lies 3′ to said tetracycline operator sequence; c) a gene sequence encoding tetracycline repressor operably linked to an HSV immediate-early promoter, wherein the gene sequence is located at the ICP0 locus; d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and e) a gene sequence encoding a functional ICP34.5 protein; wherein said oncolytic HSV does not encode functional ICP0 and does not contain a ribozyme sequence located in said 5′ untranslated region of VP5.

One aspect provided herein describes a composition comprising i) a checkpoint inhibitor; and ii) an oncolytic Herpes Simplex Virus (HSV) comprising recombinant DNA, wherein the recombinant DNA does not encode functional ICP0; and encodes fusogenic activity.

In one embodiment of any aspect described herein, the composition further comprises a pharmaceutical acceptable carrier.

In one embodiment of any aspect described herein, the composition further comprises an agent that regulates the tet operator-containing promoter.

### Definitions

All references cited herein are incorporated by reference in their entirety as though fully set forth.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. Definitions of common terms can be found in Singleton et al., Dictionary of Microbiology and Molecular Biology 3^{rd} ed., J. Wiley & Sons New York, NY (2001); March, Advanced Organic Chemistry Reactions, Mechanisms and Structure 5^{th} ed., J. Wiley & Sons New York, NY (2001); Michael Richard Green and Joseph Sambrook, Molecular Cloning: A Laboratory Manual, 4th ed., Cold Spring Harbor Laboratory Press, Cold Spring Harbor, N.Y., USA (2012); Davis et al., Basic Methods in Molecular Biology, Elsevier Science Publishing, Inc., New York, USA (2012); Jon Lorsch (ed.) Laboratory Methods in Enzymology: DNA, Elsevier, (2013); Frederick M. Ausubel (ed.), Current Protocols in Molecular Biology (CPMB), John Wiley and Sons, (2014); John E. Coligan (ed.), Current Protocols in Protein Science (CPPS), John Wiley and Sons, Inc., (2005); and Ethan M Shevach, Warren Strobe, (eds.) Current Protocols in Immunology (CPI) (John E. Coligan, ADA M Kruisbeek, David H Margulies, John Wiley and Sons, Inc., (2003); each of which provide one skilled in the art with a general guide to many of the terms used in the present application.

As used herein, a “subject” means a human or animal. Usually the animal is a vertebrate such as a primate, rodent, domestic animal or game animal. Primates include, for example, chimpanzees, cynomolgous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., cancer. A subject can be male or female.

A subject can be one who has been previously diagnosed with or identified as suffering from or having a condition in need of treatment (e.g. cancer) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ligan-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

As used herein, the term “DNA” is defined as deoxyribonucleic acid. The term “polynucleotide” is used herein interchangeably with “nucleic acid” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “operably linked,” as used herein, refers to the arrangement of various nucleic acid molecule elements relative to each other such that the elements are functionally connected and are able to interact with each other. Such elements may include, without limitation, a promoter, an enhancer, a polyadenylation sequence, one or more introns and/or exons, and a coding sequence of a gene of interest to be expressed. The nucleic acid sequence elements, when operably linked, can act together to modulate the activity of one another, and ultimately may affect the level of expression of the gene of interest, including any of those encoded by the sequences described above.

The term “vector,” as used herein, refers to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both of which are incorporated herein by reference). Additionally, the techniques described herein and demonstrated in the referenced figures are also instructive with regard to effective vector construction.

The term “oncolytic HSV-1 vector” refers to a genetically engineered HSV-1 virus corresponding to at least a portion of the genome of HSV-1 that is capable of infecting a target cell, replicating, and being packaged into HSV-1 virions. The genetically engineered virus comprises deletions and or mutations and or insertions of nucleic acid that render the virus oncolytic such that the engineered virus replicates in- and kills-tumor cells by oncolytic activity. The virus may be attenuated or non-attenuated. The virus may or may not deliver a transgene-that differs from the HSV viral genome. In one embodiment, the oncolytic HSV-1 vector does not express a transgene to produce a protein foreign to the virus.

The term “promoter,” as used herein, refers to a nucleic acid sequence that regulates, either directly or indirectly, the transcription of a corresponding nucleic acid coding sequence to which it is operably linked. The promoter may function alone to regulate transcription, or, in some cases, may act in concert with one or more other regulatory sequences such as an enhancer or silencer to regulate transcription of the gene of interest. The promoter comprises a DNA regulatory sequence, wherein the regulatory sequence is derived from a gene, which is capable of binding RNA polymerase and initiating transcription of a downstream (3′-direction) coding sequence. A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one can position the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. Depending on the promoter used, individual elements can function either cooperatively or independently to activate transcription. The promoters described herein may or may not be used in conjunction with an “enhancer,” which refers to a cis-acting regulatory sequence involved in the transcriptional activation of a nucleic acid sequence, such as those for the genes, or portions or functional equivalents thereof, listed herein.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages may be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include, the HCMV immediate-early promoter, the beta-lactamase (penicillinase), lactose and tryptophan (trp) promoter systems.

A “gene,” or a “sequence which encodes” a particular protein, is a nucleic acid molecule which is transcribed (in the case of DNA) and translated (in the case of mRNA) into a polypeptide in vitro or in vivo when placed under the control of one or more appropriate regulatory sequences. A gene of interest can include, but is no way limited to, cDNA from eukaryotic mRNA, genomic DNA sequences from eukaryotic DNA, and even synthetic DNA sequences. A transcription termination sequence will usually be located 3′ to the gene sequence. Typically, a polyadenylation signal is provided to terminate transcription of genes inserted into a recombinant virus.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26 (3): 629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

The term “oncolytic activity,” as used herein, refers to cytotoxic effects in vitro and/or in vivo exerted on tumor cells without any appreciable or significant deleterious effects to normal cells under the same conditions. The cytotoxic effects under in vitro conditions are detected by various means as known in prior art, for example, by staining with a selective stain for dead cells, by inhibition of DNA synthesis, or by apoptosis. Detection of the cytotoxic effects under in vivo conditions is performed by methods known in the art.

A “biologically active” portion of a molecule, as used herein, refers to a portion of a larger molecule that can perform a similar function as the larger molecule. Merely by way of non-limiting example, a biologically active portion of a promoter is any portion of a promoter that retains the ability to influence gene expression, even if only slightly. Similarly, a biologically active portion of a protein is any portion of a protein which retains the ability to perform one or more biological functions of the full-length protein (e.g. binding with another molecule, phosphorylation, etc.), even if only slightly.

As used herein, the term “administering,” refers to the placement of a therapeutic or pharmaceutical composition as disclosed herein into a subject by a method or route which results in at least partial delivery of the agent at a desired site. Pharmaceutical compositions comprising agents as disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of additional elements that do not materially affect the basic and novel or functional characteristic(s) of that embodiment of the technology.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

With the aforementioned preliminary descriptions and definitions in mind, additional background is provided herein below to provide context for the genesis and development of the inventive vectors, compositions and methods described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 presents data that shows therapeutic treatment of established large bilateral CT26. WT tumors in normal BALB/c mice. Female BALB/c mice, 6 to 7-weeks-old, were implanted s.c. with 5×10⁵ syngeneic CT26.WT colon cancer cells in a volume of 100 μl at both the left and right flanks. When subcutaneous tumors reached to about 180-200 mm³, mice were divided into 2 groups of 8 and 14 mice each, in which the average of tumor size in each group is essentially the same. Mice were then anesthetized and inoculated with DMEM containing 1 μg of doxycycline (n=8) or QREO5-F at 1×10⁷ PFU containing 1 μg of doxycycline in a volume of 100 ul unilaterally. The same treatment was repeated on days 3 and 6. Tumor Volumes were measured every 2 to 3 days by a caliper. Mean tumor volumes±SEM are shown. There were 8 mice in DMEM group from day 0 to day 8, and 3 mice from day 9 to day 18 post first injection. There were 14 mice in QREO5F group from day 0 to day 6, 13 mice on day 8, and 8 mice from day 9 to day 18 post first injection.

FIG. 2 presents data that shows therapeutic treatment of established large bilateral CT26.WT tumors with combination of QREO5F and atezolizumab. Large bilateral CT26.WT tumors were established in normal BALB/c mice as described above. Once the tumors reached to ˜ 90 mm³ to 180 mm³, mice were randomly divided into 4 groups, and larger tumors on one side of flanks were intratumorally injected with 100 ul of DMEM containing 1 μg of doxycycline (n=5), QREO5-F at 1×10⁷ PFU containing 1 ug doxycycline in a volume of 100 ul (n=7), or QREO5-F at 1×10⁷ PFU containing 1 ug doxycycline in a volume of 100 ul and anti-PD-L1 antibody (Atezolizumab) at 200 μg/mouse via i.p. injection (n=8). As a negative control, a group of mice (n=5) was received i.p. injection of anti-PD-L1 antibody only (200 μg/mouse). Individual groups of mice were received the same treatment on days 3 and 6 post initial inoculation. For mice receiving PD-L1 blockade, a fourth injection were carried out on day 9 post initial treatment at the same dose. Tumor Volumes were measured every 2 to 3 days by a caliper. Mean tumor volumes±SEM are shown. * p<0.05, ** p<0.01.

FIG. 3 presents data that shows induction of antitumor-specific memory response in QREO5F/anti-PD-L1 combination therapy cured mice. Four cured mice from combination therapy group of mice described in FIGS. 2 and 5 naïve female BALB/c mice were injected s.c. with 5×10e5 CT26.WT cells into the middle section between the rear left and right flanks. Tumor volumes were quantified every third day by a caliper.

### DESCRIPTION OF THE INVENTION

Cancer immunotherapy utilizes a number of different strategies for treatment of tumors. One strategy is the use of checkpoint inhibitors. These attempt to interfere with the normal inhibitory signals that regulate lymphocytes. Some examples of checkpoint inhibitors include CTLA-4, PD-1, PD-L1, PD-L2, and TIM3. By blocking the signaling capacity of these checkpoint inhibitors on tumor cells, it can result in the activation of the immune system to destroy the tumors.

Because checkpoint inhibitors rely on the activation of the patient's own immune system against tumors, its effects are not immediately evident, presenting a challenge in evaluating clinical responses to such therapy. As a result, treatment strategies usually involved the treatment of checkpoint inhibitors used in combination with additional therapeutics or treatment strategies.

### Methods of Treatment

Methods described herein are directed to the treatment of cancer via administration of a checkpoint inhibitor, and any of the oncolytic viruses described herein. For example, one aspect described herein provides a method for cancer comprising administering a subject in need thereof i) a checkpoint inhibitor; and ii) an oncolytic Herpes Simplex Virus (HSV) comprising recombinant DNA, wherein the recombinant DNA comprises: a gene comprising a 5′ untranslated region and a HSV-1, or HSV-2, VP5 gene that is operably linked to an VP5 promoter comprising a TATA element; a tetracycline operator sequence positioned between 6 and 24 nucleotides 3′ to said TATA element, wherein the VP5 gene lies 3′ to said tetracycline operator sequence; a gene sequence encoding tetracycline repressor operably linked to an HSV immediate-early promoter, wherein the gene sequence is located at the ICP0 locus; a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and a gene sequence encoding a functional ICP34.5 protein; wherein said oncolytic HSV does not encode functional ICP0 and does not contain a ribozyme sequence located in said 5′ untranslated region of VP5.

Another aspect described herein provides a method for treating cancer, the method comprising administering a subject in need thereof i) an anti-PD-L1 checkpoint inhibitor; and ii) an oncolytic Herpes Simplex Virus (HSV) comprising recombinant DNA, wherein the recombinant DNA comprises: a gene comprising a 5′ untranslated region and a HSV-1, or HSV-2, VP5 gene that is operably linked to an VP5 promoter comprising a TATA element; a tetracycline operator sequence positioned between 6 and 24 nucleotides 3′ to said TATA element, wherein the VP5 gene lies 3′ to said tetracycline operator sequence; a gene sequence encoding tetracycline repressor operably linked to an HSV immediate-early promoter, wherein the gene sequence is located at the ICP0 locus; a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and a gene sequence encoding a functional ICP34.5 protein; wherein said oncolytic HSV does not encode functional ICP0 and does not contain a ribozyme sequence located in said 5′ untranslated region of VP5.

Another aspect described herein provides a method for treating cancer, the method comprising administering a subject in need thereof i) a checkpoint inhibitor; and ii) an oncolytic Herpes Simplex Virus (HSV) comprising recombinant DNA, wherein the recombinant DNA does not encode functional ICP0; and encodes fusogenic activity.

Another aspect described herein provides a method for treating cancer, the method comprising administering a subject in need thereof i) an anti-PD-L1 checkpoint inhibitor; and ii) an oncolytic Herpes Simplex Virus (HSV) comprising recombinant DNA, wherein the recombinant DNA does not encode functional ICP0; and encodes fusogenic activity.

In one embodiment, the cancer is a solid tumor. The solid tumor can be malignant or benign. In one embodiment, the subject is diagnosed or has been diagnosed with having a carcinoma, a melanoma, a sarcoma, a germ cell tumor, and a blastoma. Exemplary cancers include, but are in no way limited to, non-small-cell lung cancer, breast cancer, brain cancer, colon cancer, prostate cancer, liver cancer, lung cancer, ovarian cancer, skin cancer, head and neck cancer, kidney cancer, and pancreatic cancer. In one embodiment, the cancer is metastatic. These types of cancers are known in the art and can be diagnosed by a skilled clinician using standard techniques known in the art, for example blood analysis, blood cell count analysis, tissue biopsy, non-invasive imaging, and/or review of family history.

In one embodiment, the checkpoint inhibitor and the oncolytic virus are administered via the same route of administration. For example, in one embodiment, the checkpoint inhibitor and the oncolytic virus are administered locally, e.g., directly to the tumor.

In one embodiment, the checkpoint inhibitor and the oncolytic virus are administered via the different routes of administration. For example, in one embodiment, the checkpoint inhibitor is administered systemically and the oncolytic virus are administered directly to the tumor.

In one embodiment, the checkpoint inhibitor and the oncolytic virus are administered in the same composition.

In one embodiment, the checkpoint inhibitor and the oncolytic virus are administered in different compositions.

In one embodiment, the checkpoint inhibitor and the oncolytic virus are administered at substantially the same time.

In one embodiment, the checkpoint inhibitor and the oncolytic virus are administered at different times. For example, in one embodiment, the checkpoint inhibitor is administered prior to administration of the oncolytic virus. In another embodiment, the checkpoint inhibitor is administered after to administration of the oncolytic virus.

In one embodiment, when the checkpoint inhibitor and the oncolytic virus are administered at different times, the administrations are separated by at least 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, or at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or longer.

### Oncolytic Virus

Oncolytic viruses are genetically modified viruses that preferentially replicate in host cancer cells, leading to the production of new viruses and ultimately, cell death. Herpes simplex virus (HSV) possesses several unique properties as an oncolytic agent. It can infect a broad range of cell types and has a short replication cycle (9 to 18 h). The use of a replication-conditional strain of HSV-1 as an oncolytic agent was first reported for the treatment of malignant gliomas. Since then, various efforts have been made in an attempt to broaden their therapeutic efficacy and increase the replication specificity of the virus in tumor cells. Not surprisingly, however, deletion of genes that impair viral replication in normal cells also leads to a marked decrease in the oncolytic activity of the virus for the targeted tumor cells. Currently, no oncolytic viruses that are able to kill only tumor cells while leaving normal cells intact are available. Consequently, the therapeutic doses of existing oncolytic viruses are significantly restricted. The availability of an oncolytic virus whose replication can be tightly controlled and adjusted pharmacologically would offer greatly increased safety and therapeutic efficacy. Such a regulatable oncolytic virus would minimize the risk of uncontrolled replication in adjacent and distant tissues as well as undesirable progeny virus overload in the target area after the tumor has been eliminated. This regulatory feature would also allow the oncolytic activity of the virus to be quickly shut down should adverse effects be detected.

HSV replicates in epithelial cells and fibroblasts and establishes life-long latent infection in neuronal cell bodies within the sensory ganglia of infected individuals. During productive infection, HSV genes fall into three major classes based on the temporal order of their expression: immediate-early (IE), early (E), and late (L) (Roizman, 2001). The HSV-1 viral proteins directly relevant to the current invention are immediate-early regulatory protein, ICP0, and the viral major capsid protein ICP5 or VP5. Although not essential for productive infection, ICP0 is required for efficient viral gene expression and replication at low multiplicities of infection in normal cells and efficient reactivation from latent infection (Cai and Schaffer, 1989; Leib et al., 1989; Yao and Schaffer, 1995). ICP0 is needed to stimulate translation of viral mRNA in quiescent cells (Walsh and Mohr, 2004) and plays a fundamental role in counteracting host innate antiviral response to HSV infection. In brief, it prevents an IFN-induced nuclear block to viral transcription, down regulates TLR2/TLR9-induced inflammatory cytokine response to viral infection, suppresses TNF-α mediated activation of NF-κB signaling pathway, and interferes with DNA damage response to viral infection (Lanfranca et al., 2014). Given that tumor cells are impaired in various cellular pathways, such as DNA repair, interferon signaling, and translation regulation (Barber, 2015; Critchley-Thorne et al., 2009; Kastan and Bartek, 2004; Li and Chen, 2018; Mohr, 2005; Zitvogel et al., 2015), it is not surprising that ICP0 deletion mutants replicate much more efficiently in cancer cells than in normal cells, in particular, quiescent cells and terminally differentiated cells. The oncolytic potential of ICP0 mutants was first illustrated by Yao and Schaffer (Yao and Schaffer, 1995), who showed that the plaque-forming efficiency of an ICP0 null mutant in human osteoscarcoma cells (U2OS) is 100- to 200-fold higher than in non-tumorigenic African green monkey kidney cells (Vero). It has been recently shown the defect in stimulator of interferon genes (STING) signaling pathway in U2OS cells leads to its demonstrated ability to efficiently support the growth of ICP0 null mutant (Deschamps and Kalamvoki, 2017).

Using the T-REx™ gene switch technology (Thermo Fisher/Invitrogen, Carlsbad, CA) invented by Dr. Feng Yao and a self-cleaving ribozyme, the first regulatable oncolytic virus, KTR27 (U.S. Pat. No. 8,236,941, which is incorporated herein by reference in its entirety), in which the HSV-1 ICP0 gene is replaced by DNA sequence encoding tetracycline repressor (tetR) was created, while the essential HSV-1 ICP27 gene is controlled by the tetO-bearing ICP27 promoter and a self-cleaving ribozyme in the 5′ untranslated region of the ICP27 coding sequence. Recent DNA sequence analyses of a KTR27-derived fusogenic virus, named KTR27-F, indicates that in addition to the deletion of both copies of ICP0 gene, both copies of HSV-1 ICP34.5 gene are also deleted from the said KTR27-F virus. Moreover, PCR analyses of KTR27 viral DNA with the ICP34.5 gene-specific primers has revealed that like KTR27-F, KTR27 does not encode ICP0 gene and ICP34.5 gene. ICP34.5 gene is located 5′ to the ICP0 gene in the inverted repeat region of HSV-1 genome that flanks the unique long sequence of HSV-1 genome. Various HSV-1 onclytic viruses are based on the deletion of ICP34.5 gene (Aghi and Martuza, 2005; Kaur et al., 2012; Lawler et al., 2017), including the recently FDA-approved talimogene laherparepvec (T-VEC) for treatment of advanced-stage melanoma (Rehman et al., 2016).

Building on the tet-dependent viral replication and onco-selectivity profiles of KTR27 and the notion that the self-cleaving ribozyme employed in construction of KTR27 for achieving higher degree of tet-dependent viral replication significantly restricts viral replication in cancer cells because of less than optimal expression of ICP27, a new ICP0 null mutant-based tetR-expressing oncolytic virus QREO5 that encodes the late HSV-1 major capsid protein VP5 under the control of the tetO-containing VP5 promoter was recently developed. Because VP5 is a late viral gene product, whose expression is dependent on the expression of viral IE genes, it was hypothesized that the late kinetics of the tetO-bearing VP5 promoter would allow for more stringent control of VP5 expression than that of ICP27 under the control of the tetO-bearing ICP27 promoter by tetR expressed from the IE ICP0 promoter. Indeed, QREO5 exhibits significantly superior tet-dependent viral replication than KTR27 in infected H1299 cells and Vero cells. Moreover, because the QREO5 genome contains no self-cleaving ribozyme and encodes wild-type ICP34.5 gene, it replicates 100- and 450-fold more efficiently than KTR27 in Vero cells and H1299 cells, respectively.

HSV-1 is a human neurotropic virus that is capable of infecting virtually all vertebrate cells. Natural infections follow either a lytic, replicative cycle or establish latency, usually in peripheral ganglia, where the DNA is maintained indefinitely in an episomal state. HSV-1 contains a double-stranded, linear DNA genome, about 152 kilobases in length, which has been completely sequenced by McGeoch (McGeoch et al., J. Gen. Virol. 69:1531 (1988); McGeoch et al., Nucleic Acids Res 14:1727 (1986); McGeoch et al., J. Mol. Biol. 181:1 (1985); Perry and McGeoch, J. Gen. Virol. 69:2831 (1988); Szpara M L et al., J Virol. 2010, 84:5303; Macdonald S J et al., J Virol. 2012, 86:6371). DNA replication and virion assembly occurs in the nucleus of infected cells. Late in infection, concatemeric viral DNA is cleaved into genome length molecules which are packaged into virions. In the CNS, herpes simplex virus spreads transneuronally followed by intraaxonal transport to the nucleus, either retrograde or anterograde, where replication occurs.

Accordingly, described herein is an oncolytic Herpes Simplex Virus (HSV) comprising recombinant DNA, wherein the recombinant DNA comprises: a gene comprising a 5′ untranslated region and a HSV-1, or HSV-2, VP5 gene that is operably linked to an VP5 promoter comprising a TATA element; a tetracycline operator sequence positioned between 6 and 24 nucleotides 3′ to said TATA element, wherein the VP5 gene lies 3′ to said tetracycline operator sequence; a gene sequence encoding tetracycline repressor operably linked to an HSV immediate-early promoter, wherein the gene sequence is located at the ICP0 locus; a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and a gene sequence encoding a functional ICP34.5 protein, wherein said oncolytic HSV does not encode functional ICP0 and does not contain a ribozyme sequence located in said 5′ untranslated region of VP5. In one embodiment, the recombinant DNA is derived from the HSV-1 genome. In an alternative embodiment, the recombinant DNA is derived from the HSV-2 genome. In one embodiment, the genome of the HSV comprising recombinant DNA consists of, consists essentially of, or comprises the sequence of SEQ ID NO: 1.

A distinguishing feature of the oncolytic virus described herein is that the viral genome expression a gene sequence that encodes functional ICP34.5. Infected cell protein 34.5 (ICP34.5) is a protein (e.g., a gene product) expressed by the γ34.5 gene in viruses, such as the herpes simplex virus. ICP34.5 is one of HSV neurovirulence factors (Chou J, Kern E R, Whitley R J, and Roizman B, Science, 1990). One of the functions of ICP34.5 is to block the cellar stress response to a viral infection, i.e., blocking the double-stranded RNA-dependent protein kinase PKR-mediated antiviral response (Agarwalla, P. K., et al. Method in Mol. Bio., 2012).

The oncolytic virus described herein is a ICP0 null virus. Infected cell polypeptide 0 (ICP0) is a protein encoded by the HSV-1 α0 gene. ICP0 is generated during the immediate-early phase of viral gene expression. ICP0 is synthesized and transported to the nucleus of the infected host cell, where it promotes transcription from viral genes, disrupts nuclear and cytoplasmic cellular structures, such as the microtubule network, and alters the expression of host genes. One skilled in the art can determine if the ICP0 gene product has been deleted or if the virus does not express functional forms of this gene product using PCR-based assays to detect the presence of the gene in the viral genome or the expression of the gene products, or using functional assays to assess their function, respectively.

In one embodiment, the gene that encodes these gene products contain a mutation, for example, an inactivating mutation, that inhibits proper expression of the gene product. For example, the gene may encode a mutation in the gene product that inhibits proper folding, expression, function, etc. of the gene product. As used herein, the term “inactivating mutation” is intended to broadly mean a mutation or alteration to a gene wherein the expression of that gene is significantly decreased, or wherein the gene product is rendered nonfunctional, or its ability to function is significantly decreased. The term “gene” encompasses both the regions coding the gene product as well as regulatory regions for that gene, such as a promoter or enhancer, unless otherwise indicated.

Ways to achieve such alterations include: (a) any method to disrupt the expression of the product of the gene or (b) any method to render the expressed gene nonfunctional. Numerous methods to disrupt the expression of a gene are known, including the alterations of the coding region of the gene, or its promoter sequence, by insertions, deletions and/or base changes. (See, Roizman, B. and Jenkins, F. J., Science 229:1208-1214 (1985)).

An essential feature of the DNA of the present invention is the presence of a gene needed for virus replication that is operably linked to a promoter having a TATA element. A tet operator sequence is located between 6 and 24 nucleotides 3′ to the last nucleotide in the TATA element of the promoter and 5′ to the gene. The strength with which the tet repressor binds to the operator sequence is enhanced by using a form of operator which contains two op2 repressor binding sites (each such site having the nucleotide sequence: TCCCTATCAGTGATAGAGA (SEQ ID NO: 3)) linked by a sequence of 2-20, preferably 1-3 or 10-13, nucleotides. When repressor is bound to this operator, very little or no transcription of the associated gene will occur. If DNA with these characteristics is present in a cell that also expresses the tetracycline repressor, transcription of the gene will be blocked by the repressor binding to the operator and replication of the virus will not occur. However, if tetracycline, for example, is introduced, it will bind to the repressor, cause it to dissociate from the operator, and virus replication will proceed.

During productive infection, HSV gene expression falls into three major classes based on the temporal order of expression: immediate-early (a), early (B), and late (Y), with late genes being further divided into two groups, γ1 and γ2. The expression of immediate-early genes does not require de novo viral protein synthesis and is activated by the virion-associated protein VP16 together with cellular transcription factors when the viral DNA enters the nucleus. The protein products of the immediate-early genes are designated infected cell polypeptides ICP0, ICP4, ICP22, ICP27, and ICP47 and it is the promoters of these genes that are preferably used in directing the expression of tet repressor (tetR). The expression of a gene needed for virus replication is under the control of the tetO-containing promoters and these essential genes may be immediate-early, early or late genes, e.g., ICP4, ICP27, ICP8, UL9, gD and VP5. In one embodiment, the tetR has the sequence of SEQ ID NO: 4.

ICP0 plays a major role in enhancing the reactivation of HSV from latency and confers a significant growth advantage on the virus at low multiplicities of infection. ICP4 is the major transcriptional regulatory protein of HSV-1, which activates the expression of viral early and late genes. ICP27 is essential for productive viral infection and is required for efficient viral DNA replication and the optimal expression of subset of viral β genes and γ1 genes as well as viral γ2 genes. The function of ICP47 during HSV infection appears to be to down-regulate the expression of the major histocompatibility complex (MHC) class I on the surface of infected cells.

The recombinant DNA may also include at least one, and preferably at least two, sequences coding for the tetracycline repressor with expression of these sequences being under the control of an immediate early promoter, preferably ICP0 or ICP4. The sequence for the HSV ICP0 and ICP4 promoters and for the genes whose regulation they endogenously control are well known in the art (Perry, et al., J. Gen. Virol. 67:2365-2380 (1986); McGeoch et al., J. Gen. Virol. 72:3057-3075 (1991); McGeoch et al., Nucl. Acid Res. 14:1727-1745 (1986)) and procedures for making viral vectors containing these elements have been previously described (see US published application 2005-0266564).

These promoters are not only very active in promoting gene expression, they are also specifically induced by VP16, a transactivator released when HSV-1 infects a cell. Thus, transcription from ICP0 promoter is particularly high when repressor is most needed to shut down virus replication. Once appropriate DNA constructs have been produced, they may be incorporated into HSV-1 virus using methods that are well known in the art. One appropriate procedure is described in US 2005-0266564 but other methods known in the art may also be employed.

In various embodiments, the variant gene comprises at least one amino acid change that deviates from the wild-type sequence of the gene. In one embodiment, an oncolytic HSV described herein can contain two or more amino acid substitutions in at least one variant gene. The at least two amino acid substitutions can be found in the same gene, for example, the gK variant gene contains at least two amino acid substitutions. Alternatively, the at least two amino acid substitutions can be found in the at least two different genes, for example, the gK variant gene and the UL24 variant gene each contains at least one amino acid substitutions.

SEQ ID NO: 2 is the amino acid sequence encoding
gK (strain KOS).
(SEQ ID NO: 2)
MLAVRSLQHLSTVVLITAYGLVLVWYTVFGASPLHRCIYAVRPTGTNNDT
ALVWMKMNQTLLFLGAPTHPPNGGWRNHAHICYANLIAGRVVPFQVPPDA
TNRRIMNVHEAVNCLETLWYTRVRLVVVGWFLYLAFVALHQRRCMFGVVS
PAHKMVAPATYLLNYAGRIVSSVFLQYPYTKITRLLCELSVQRQNLVQLF
ETDPVTFLYHRPAIGVIVGCELMLRFVAVGLIVGTAFISRGACAITYPLF
LTITTWCFVSTIGLTELYCILRRGPAPKNADKAAAPGRSKGLSGVCGRCC
SIILSGIAMRLCYIAVVAGVVLVALHYEQEIQRRLFDV

Another distinguishing feature of the oncolytic virus described herein is that the viral genome sequence does not contain a ribozyme sequence, for example, at the 5′ untranslated region of VP5. A ribozyme is an RNA molecule that is capable of catalyzing a biochemical reaction in a similar manner as a protein enzyme. Ribozymes are further described in, e.g., Yen et al., Nature 431:471-476, 2004, the contents of which are incorporated herein by reference in its entirety.

In one embodiment, the oncolytic HSV described herein further encodes fusogenic activity.

Another aspect provides an oncolytic Herpes Simplex Virus (HSV) comprising recombinant DNA that does not encode functional ICP0 and encodes fusogenic activity.

### Checkpoint Inhibitors

Methods and compositions described herein require the use of a checkpoint inhibitor in combination with any of the oncolytic viruses described herein. In some embodiments of any of the aspects, a checkpoint inhibitor can be a small molecule, inhibitory RNA/RNAi molecule (both single and double stranded), an antibody, antibody reagent, or an antigen-binding fragment thereof that specifically binds to at least one immune checkpoint protein. Common checkpoints that are targeted for therapeutics include, but are not limited to PD-1, CTLA4, TIM3, LAG3 and PD-L1. Inhibitors of their checkpoint regulators are known in the art.

Non-limiting examples of checkpoint inhibitors (with checkpoint targets and manufacturers noted in parentheses) can include: MGA271 (B7-H3: MacroGenics); ipilimumab (CTLA-4; Bristol Meyers Squibb); pembrolizumab (PD-1; Merck); nivolumab (PD-1; Bristol Meyers Squibb); atezolizumab (PD-L1; Genentech); galiximab (B7.1; Biogen); IMP321 (LAG3: Immuntep); BMS-986016 (LAG3; Bristol Meyers Squibb); SMB-663513 (CD137; Bristol-Meyers Squibb); PF-05082566 (CD137; Pfizer); IPH2101 (KIR; Innate Pharma); KW-0761 (CCR4; Kyowa Kirin); CDX-1127 (CD27; CellDex); MEDI-6769 (Ox40; MedImmune); CP-870,893 (CD40; Genentech); tremelimumab (CTLA-4; Medimmune); pidilizumab (PD-1; Medivation); MPDL3280A (PD-L1; Roche); MEDI4736 (PD-L1; AstraZeneca); MSB0010718C (PD-L1; EMD Serono); AUNP12 (PD-1; Aurigene); avelumab (PD-L1; Merck); durvalumab (PD-L1; Medimmune); TSR-022 (TIM3; Tesaro).

Programmed cell death 1 (PD-1) limits the activity of T cells in peripheral tissues at the time of an inflammatory response to infection and limits autoimmunity. PD-1 blockade in vitro enhances T-cell proliferation and cytokine production in response to a challenge by specific antigen targets or by allogeneic cells in mixed lymphocyte reactions. A strong correlation between PD-1 expression and response was shown with blockade of PD-1 (Pardoll, Nature Reviews Cancer, 12:252-264, 2012). PD-1 blockade can be accomplished by a variety of mechanisms including antibodies that bind PD-1 or its ligand, PD-L1. Examples of PD-1 and PD-L1 blockers are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699; which are incorporated by reference herein in their entireties. In certain embodiments the PD-1 inhibitors include anti-PD-L1 antibodies. PD-1 inhibitors include anti-PD-1 antibodies and similar binding proteins such as anti-PD-1 antibody clone RMP1-14, nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224, a fusion protein of B7-DC; an antibody Fc portion; BMS-936559 (MDX-1105-01) for PD-L1 (B7-H1) blockade.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies.

In some embodiments, the antibody or antigen-binding portion thereof is a fully human antibody. In some embodiments, the antibody, antigen-binding portion thereof, is a humanized antibody or antibody reagent. In some embodiments, the antibody, antigen-binding portion thereof, is a fully humanized antibody or antibody reagent. In some embodiments, the antibody or antigen-binding portion thereof, is a chimeric antibody or antibody reagent. In some embodiments, the antibody, antigen-binding portion thereof, is a recombinant polypeptide. In some embodiments, the chimeric T cell antigen receptor comprises an extracellular domain that binds EGFRvIII, wherein the extracellular domain comprises a humanized or chimeric antibody or antigen-binding portion thereof.

The term “human antibody” refers to antibodies whose variable and constant regions correspond to or are derived from immunoglobulin sequences of the human germ line, as described, for example, by Kabat et al. (see Kabat, et al. (1991) Sequences of Proteins of Immunological Interest, Fifth Edition, U.S. Department of Health and Human Services, NIH Publication No. 91-3242). However, the human antibodies can contain amino acid residues not encoded by human germ line immunoglobulin sequences (for example mutations which have been introduced by random or site-specific mutagenesis in vitro or by somatic mutation in vivo), for example in the CDRs, and in particular in CDR3. Recombinant human antibodies as described herein have variable regions and may also contain constant regions derived from immunoglobulin sequences of the human germ line (see Kabat, E. A., et al. (1991) Sequences of Proteins of Immunological Interest, Fifth Edition, U.S. Department of Health and Human Services, NIH Publication No. 91-3242). According to particular embodiments, however, such recombinant human antibodies are subjected to in-vitro mutagenesis (or to a somatic in-vivo mutagenesis, if an animal is used which is transgenic due to human Ig sequences) so that the amino acid sequences of the VH and VL regions of the recombinant antibodies are sequences which although related to or derived from VH and VL sequences of the human germ line, do not naturally exist in vivo within the human antibody germ line repertoire. According to particular embodiments, recombinant antibodies of this kind are the result of selective mutagenesis or back mutation or of both. Preferably, mutagenesis leads to an affinity to the target which is greater, and/or an affinity to non-target structures which is smaller than that of the parent antibody. Generating a humanized antibody from the sequences and information provided herein can be practiced by those of ordinary skill in the art without undue experimentation. In one approach, there are four general steps employed to humanize a monoclonal antibody, see, e.g., U.S. Pat. Nos. 5,585,089; 6,835,823; 6,824,989. These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

Usually the CDR regions in humanized antibodies and human antibody variants are substantially identical, and more usually, identical to the corresponding CDR regions in the mouse or human antibody from which they were derived. In some embodiments, it is possible to make one or more conservative amino acid substitutions of CDR residues without appreciably affecting the binding affinity of the resulting humanized immunoglobulin or human antibody variant. In some embodiments, substitutions of CDR regions can enhance binding affinity.

In some embodiments, the antibody is a nanobody. As used herein, a “nanobody” refers to a single-domain antibody comprising a single monomeric variable antibody domain. A nanobody selectively binds to a specific antigen, similar to an antibody. A nanobody is typically small in size, ranging from 12-15 kDa. Methods for designing and producing nanobodies are known in the art and are further described in Ghahroudi, et al. FEBS Letters. September 1997, 414:3 (521-526), which is incorporated herein in its entirety by reference.

Antibodies or antibody reagents that bind to PD-1, or its ligand PD-L1, are described in, e.g., U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699; which are incorporated by reference herein in their entireties. In certain embodiments the PD-1 antibodies include nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224, a fusion protein of B7-DC; an antibody Fc portion; BMS-936559 (MDX-1105-01) for PD-L1 (B7-H1) blockade. Also specifically contemplated herein are agents that disrupt or block the interaction between PD-1 and PD-L1, such as a high affinity PD-L1 antagonist.

Non-limiting examples of PD-1 antibodies include: pembrolizumab (Merck); nivolumab (Bristol Meyers Squibb); pidilizumab (Medivation); and AUNP12 (Aurigene). Non-limiting examples of PD-L1 antibodies can include atezolizumab (Genentech); MPDL3280A (Roche); MEDI4736 (AstraZeneca); MSB0010718C (EMD Serono); avelumab (Merck); and durvalumab (Medimmune).

Antibodies that bind to OX40 (also known as CD134), are described in, e.g., US Patent Nos. U.S. Pat. Nos. 9,006,399, 9,738,723, 9,975,957, 9,969,810, 9,828,432; PCT Published Patent Application Nos: WO2015153513, WO2014148895, WO2017021791, WO2018002339; and US Application Nos: US20180273632; US20180237534; US20180230227; US20120269825; which are incorporated by reference herein in their entireties.

Antibodies that bind to CTLA-4, are described in, e.g., US Patent Nos. U.S. Pat. Nos. 9,714,290, 6,984,720, 7,605,238, 6,682,736, 7,452,535; PCT Published Patent Application No: WO2009100140; and US Application Nos: US20090117132A, US20030086930, US20050226875, US20090238820; which are incorporated by reference herein in their entireties. Non-limiting examples of CTLA-4 antibodies include: ipilimumab (Bristol-Myers Squibb)

Antibodies that bind to TIM3, are described in, e.g., US Patent Nos. U.S. Pat. Nos. 8,552,156, 9,605,070, 9,163,087, 8,329,660; PCT Published Patent Application No: WO2018036561, WO2017031242, WO2017178493; and US Application Nos: US20170306016, US20150110792, US20180057591, US20160200815; which are incorporated by reference herein in their entireties.

Antibodies that bind to TIGIT (also known as CD134), are described in, e.g., US Patent Nos. U.S. Pat. Nos. 10,017,572, 9,713,641; PCT Published Patent Application No: WO2017030823; and US Application Nos: US20160355589, US20160176963, US20150322119; which are incorporated by reference herein in their entireties.

### Compositions

One aspect of the invention described herein provides a composition comprising i) a checkpoint inhibitor and ii) any of the oncolytic HSV described herein. For example, an oncolytic Herpes Simplex Virus (HSV) comprising recombinant DNA, wherein the recombinant DNA comprises: a gene comprising a 5′ untranslated region and a HSV-1, or HSV-2, VP5 gene that is operably linked to an VP5 promoter comprising a TATA element; a tetracycline operator sequence positioned between 6 and 24 nucleotides 3′ to said TATA element, wherein the VP5 gene lies 3′ to said tetracycline operator sequence; a gene sequence encoding tetracycline repressor operably linked to an HSV immediate-early promoter, wherein the gene sequence is located at the ICP0 locus; a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and a gene sequence encoding a functional ICP34.5 protein; wherein said oncolytic HSV does not encode functional ICP0 and does not contain a ribozyme sequence located in said 5′ untranslated region of VP5.

Another aspect of the invention described herein provides a composition comprising i) a checkpoint inhibitor and ii) an oncolytic Herpes Simplex Virus (HSV) comprising recombinant DNA, wherein the recombinant DNA does not encode functional ICP0; and encodes fusogenic activity.

In one embodiment, the composition is a pharmaceutical composition. As used herein, the term “pharmaceutical composition” refers to the active agent in combination with a pharmaceutically acceptable carrier e.g. a carrier commonly used in the pharmaceutical industry.

In one embodiment, the composition further comprises at least one pharmaceutically acceptable carrier. Pharmaceutically acceptable carriers are well known in the art and include aqueous solutions such as physiologically buffered saline or other solvents or vehicles such as glycols, glycerol, vegetable oils (e.g., olive oil) or injectable organic esters. A pharmaceutically acceptable carrier can be used to administer the compositions of the invention to a cell in vitro or to a subject in vivo. A pharmaceutically acceptable carrier can contain a physiologically acceptable compound that acts, for example, to stabilize the composition or to increase the absorption of the agent. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. One skilled in the art would know that the choice of a pharmaceutically acceptable carrier, including a physiologically acceptable compound, depends, for example, on the route of administration of the oncolytic HSV.

In one embodiment, the composition further comprises an agent that regulates the tet operator-containing promoter; e.g., doxycycline or tetracycline.

### Kits

One aspect of the invention described herein provides a kit comprising i) a checkpoint inhibitor and ii) any of the oncolytic HSV described herein. For example, an oncolytic Herpes Simplex Virus (HSV) comprising recombinant DNA, wherein the recombinant DNA comprises: a gene comprising a 5′ untranslated region and a HSV-1, or HSV-2, VP5 gene that is operably linked to an VP5 promoter comprising a TATA element; a tetracycline operator sequence positioned between 6 and 24 nucleotides 3′ to said TATA element, wherein the VP5 gene lies 3′ to said tetracycline operator sequence; a gene sequence encoding tetracycline repressor operably linked to an HSV immediate-early promoter, wherein the gene sequence is located at the ICP0 locus; a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and a gene sequence encoding a functional ICP34.5 protein; wherein said oncolytic HSV does not encode functional ICP0 and does not contain a ribozyme sequence located in said 5′ untranslated region of VP5.

Another aspect of the invention described herein provides a kit comprising i) a checkpoint inhibitor and ii) an oncolytic Herpes Simplex Virus (HSV) comprising recombinant DNA, wherein the recombinant DNA does not encode functional ICP0; and encodes fusogenic activity.

Kits typically include a label indicating the intended use of the contents of the kit. Associated with such a kit can be a notice in the form prescribed by a governmental agency regulating the manufacture, use or sale of pharmaceuticals or biological products, which notice reflects approval by the agency of manufacture, use or sale for human administration.

A kit described herein can optionally comprise at least one additional reagent (e.g., standards, markers and the like). In one embodiment, the kit further comprises an agent that regulates the tet operator-containing promoter; e.g., doxycycline or tetracycline.

### Administrations

The oncolytic viruses described herein or composition thereof can be administered to a subject having cancer. In one embodiment, an agent that regulates the tet operator is further administered with the oncolytic viruses described herein or composition thereof. Exemplary agents include, but are not limited to, doxycycline or tetracycline.

In cases where tumors are readily accessible, e.g., tumors of the skin, mouth or which are accessible as the result of surgery, virus can be applied topically. In other cases, it can be administered by injection or infusion. The agent that regulates the tet operator and tetR interaction, for example doxycycline or tetracycline, used prior to infection or at a time of infection can also be administered in this way or it can be administered systemically, for example, orally.

Although certain routes of administration are provided in the foregoing description, according to the invention, any suitable route of administration of the vectors may be adapted, and therefore the routes of administration described above are not intended to be limiting. Routes of administration may include, but are not limited to, intravenous, regional artery infusion, oral, buccal, intranasal, inhalation, topical application to a mucosal membrane or injection, including intratumoral, intradermal, intrathecal, intracisternal, intralesional or any other type of injection. Administration can be effected continuously or intermittently and will vary with the subject and the condition to be treated. One of skill in the art would readily appreciate that the various routes of administration described herein would allow for the inventive vectors or compositions to be delivered on, in, or near the tumor or targeted cancer cells. One of skill in the art would also readily appreciate that various routes of administration described herein will allow for the vectors and compositions described herein to be delivered to a region in the vicinity of the tumor or individual cells to be treated. “In the vicinity” can include any tissue or bodily fluid in the subject that is in sufficiently close proximity to the tumor or individual cancer cells such that at least a portion of the vectors or compositions administered to the subject reach their intended targets and exert their therapeutic effects.

Prior to administration, the oncolytic viruses can be suspended in any pharmaceutically acceptable solution including sterile isotonic saline, water, phosphate buffered saline, 1,2-propylene glycol, polyglycols mixed with water, Ringer's solution, etc. The exact number of viruses to be administered is not crucial to the invention but should be an “effective amount,” i.e., an amount sufficient to cause cell lysis extensive enough to generate an immune response to released tumor antigens. Since virus is replicated in the cells after infection, the number initially administered will increase rapidly with time. Thus, widely different amounts of initially administered virus can give the same result by varying the time that they are allowed to replicate, i.e., the time during which cells are exposed to tetracycline. In general, it is expected that the number of viruses (PFU) initially administered will be between 1×10⁶ and 1×10¹⁰.

Tetracycline or doxycycline will be administered either locally or systemically to induce viral replication at a time of infection or 1-72 h prior to infection. The amount of tetracycline or doxycycline to be administered will depend upon the route of delivery. In vitro, 1 μg/ml of tetracycline is more than sufficient to allow viral replication in infected cells. Thus, when delivered locally, a solution containing anywhere from 0.1 μg/ml to 100 μg/ml may be administered. However, much higher doses of tetracycline or doxycycline (e.g., 1-5 mg/ml) can be employed if desired. The total amount given locally at a single time will depend on the size of the tumor or tumors undergoing treatment but in general, it is expected that between 0.5 and 200 ml of tetracycline or doxycycline solution would be used at a time. When given systemically, higher doses of tetracycline or doxycycline will be given but it is expected that the total amount needed will be significantly less than that typically used to treat bacterial infections (for example, with doxycycline, usually 1-2 grams per day in adults divided into 2-4 equal doses and, in children, 2.2-4.4 mg per kilogram of body weight, which can be divided into at least 2 doses, per day). It is expected that 5-100 mg per day should be effective in most cases. Dosing for tetracycline and doxycycline are well known in the art and can best be determined by a skilled clinician for a given patient.

The effectiveness of a dosage, as well as the effectiveness of the overall treatment can be assessed by monitoring tumor size using standard imaging techniques over a period of days, weeks and/or months. A shrinkage in the size or number of tumors is an indication that the treatment has been successful. If this does not occur or continue, then the treatment can be repeated as many times as desired. In addition, treatment with virus can be combined with any other therapy typically used for solid tumors, including surgery, radiation therapy or chemotherapy. In addition, the procedure can be combined with methods or compositions designed to help induce an immune response.

As used herein, the term “therapeutically effective amount” is intended to mean the amount of vector which exerts oncolytic activity, causing attenuation or inhibition of tumor cell proliferation, leading to tumor regression. An effective amount will vary, depending upon the pathology or condition to be treated, by the patient and his or her status, and other factors well known to those of skill in the art. Effective amounts are easily determined by those of skill in the art. In some embodiments a therapeutic range is from 10³ to 10¹² plaque forming units introduced once. In some embodiments a therapeutic dose in the aforementioned therapeutic range is administered at an interval from every day to every month via the intratumoral, intrathecal, convection-enhanced, intravenous or intra-arterial route.

The invention provided herein can further be described in the following numbered paragraphs.
1. A method for treating cancer, the method comprising administering a subject in need thereof,
i) a checkpoint inhibitor; and
ii) an oncolytic Herpes Simplex Virus (HSV) comprising recombinant DNA, wherein the recombinant DNA comprises:
a) a gene comprising a 5′ untranslated region and a HSV-1, or HSV-2, VP5 gene that is operably linked to an VP5 promoter comprising a TATA element;
b) a tetracycline operator sequence positioned between 6 and 24 nucleotides 3′ to said TATA element, wherein the VP5 gene lies 3′ to said tetracycline operator sequence;
c) a gene sequence encoding tetracycline repressor operably linked to an HSV immediate-early promoter, wherein the gene sequence is located at the ICP0 locus;
d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and
e) a gene sequence encoding a functional ICP34.5 protein;
wherein said oncolytic HSV does not encode functional ICP0 and does not contain a ribozyme sequence located in said 5′ untranslated region of VP5.
2. The method of paragraph 1, wherein the cancer is a solid tumor.
3. The method of any preceding paragraph, wherein the tumor is benign.
4. The method of any preceding paragraph, wherein the tumor is malignant.
5. The method of any preceding paragraph, wherein the subject is diagnosed or has been diagnosed as having cancer is selected from the list consisting of: a carcinoma, a melanoma, a sarcoma, a germ cell tumor, and a blastoma.
6. The method of any preceding paragraph, wherein the subject is diagnosed or has been diagnosed as having a cancer selected from the group consisting of: non-small-cell lung cancer, breast cancer, brain cancer, colon cancer, prostate cancer, liver cancer, lung cancer, ovarian cancer, skin cancer, head and neck cancer, kidney cancer, and pancreatic cancer.
7. The method of any preceding paragraph, wherein the cancer is metastatic.
8. The method of any preceding paragraph, wherein the variant gene is a gK variant gene that encodes an amino acid substitution selected from the group consisting of: an Ala to Thr amino acid substitution corresponding to amino acid 40 of SEQ ID NO: 2; an Ala to “x” amino acid substitution corresponding to amino acid 40 of SEQ ID NO: 2, wherein “x” is any amino acid; an Asp to Asn amino acid substitution corresponding to amino acid 99 of SEQ ID NO: 2; a Leu to Pro amino acid substitution corresponding to amino acid 304 of SEQ ID NO: 2; and an Arg to Leu amino acid substitution corresponding to amino acid 310 of SEQ ID NO: 2.
9. The method of any preceding paragraph, wherein the tetracycline operator sequence comprises two Op2 repressor binding sites.
10. The method of any preceding paragraph, wherein the VP5 promoter is an HSV-1 or HSV-2 VP5 promoter.
11. The method of any preceding paragraph, wherein the immediate-early promoter is an HSV-1 or HSV-2 immediate-early promoter.
12. The method of any preceding paragraph, wherein the HSV immediate-early promoter is selected from the group consisting of: ICP0 promoter, ICP27 promoter and ICP4 promoter.
13. The method of any preceding paragraph, wherein the recombinant DNA is part of the HSV-1 genome.
14. The method of any preceding paragraph, wherein the recombinant DNA is part of the HSV-2 genome.
15. The method of any preceding paragraph, further comprising administering an agent that regulates the tet operator-containing promoter.
16. The method of any preceding paragraph, wherein the agent is doxycycline or tetracycline.
17. The method of any preceding paragraph, wherein the agent is administered locally or systemically.
18. The method of any preceding paragraph, wherein the systemic administration is oral administration.
19. The method of any preceding paragraph, wherein the checkpoint inhibitor and the oncolytic virus are administered directly to the tumor.
20. The method of any preceding paragraph, wherein the checkpoint inhibitor is administered systemically and the oncolytic virus are administered directly to the tumor.
21. The method of any preceding paragraph, wherein the checkpoint inhibitor and the oncolytic virus are administered in the same composition.
22. The method of any preceding paragraph, wherein the checkpoint inhibitor and the oncolytic virus are administered in different compositions.
23. The method of any preceding paragraph, wherein the checkpoint inhibitor and the oncolytic virus are administered at substantially the same time.
24. The method of any preceding paragraph, wherein the checkpoint inhibitor and the oncolytic virus are administered at different times.
25. The method of any preceding paragraph, wherein the checkpoint inhibitor is selected from the group consisting of: an anti-PD-1 antibody or antibody reagent, an anti-PD-L1 antibody or antibody reagent, an anti-OX40 antibody or antibody reagent, a CTLA-4 antibody or antibody reagent, a TIM-3 antibody or antibody reagent, and a TIGIT antibody or antibody reagent.
26. A method for treating cancer, the method comprising administering a subject in need thereof,
i) an anti-PD-L1 checkpoint inhibitor; and
ii) an oncolytic Herpes Simplex Virus (HSV) comprising recombinant DNA, wherein the recombinant DNA comprises:
a) a gene comprising a 5′ untranslated region and a HSV-1, or HSV-2, VP5 gene that is operably linked to an VP5 promoter comprising a TATA element;
b) a tetracycline operator sequence positioned between 6 and 24 nucleotides 3′ to said TATA element, wherein the VP5 gene lies 3′ to said tetracycline operator sequence;
c) a gene sequence encoding tetracycline repressor operably linked to an HSV immediate-early promoter, wherein the gene sequence is located at the ICP0 locus;
d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and
e) a gene sequence encoding a functional ICP34.5 protein;
wherein said oncolytic HSV does not encode functional ICP0 and does not contain a ribozyme sequence located in said 5′ untranslated region of VP5.
27. A method for treating cancer, the method comprising administering a subject in need thereof,
i) a checkpoint inhibitor; and
ii) an oncolytic Herpes Simplex Virus (HSV) comprising recombinant DNA, wherein the recombinant DNA does not encode functional ICP0; and encodes fusogenic activity.
28. A kit comprising
i) a checkpoint inhibitor; and
ii) an oncolytic Herpes Simplex Virus (HSV) comprising recombinant DNA, wherein the recombinant DNA comprises:
a) a gene comprising a 5′ untranslated region and a HSV-1, or HSV-2, VP5 gene that is operably linked to an VP5 promoter comprising a TATA element;
b) a tetracycline operator sequence positioned between 6 and 24 nucleotides 3′ to said TATA element, wherein the VP5 gene lies 3′ to said tetracycline operator sequence;
c) a gene sequence encoding tetracycline repressor operably linked to an HSV immediate-early promoter, wherein the gene sequence is located at the ICP0 locus;
d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and
e) a gene sequence encoding a functional ICP34.5 protein;
wherein said oncolytic HSV does not encode functional ICP0 and does not contain a ribozyme sequence located in said 5′ untranslated region of VP5.
29. A kit comprising
i) a checkpoint inhibitor; and
ii) an oncolytic Herpes Simplex Virus (HSV) comprising recombinant DNA, wherein the recombinant DNA does not encode functional ICP0; and encodes fusogenic activity.
30. The kit of any preceding paragraph, further comprising an agent that regulates the tet operator-containing promoter.
31. The kit of any preceding paragraph, wherein the agent is doxycycline or tetracycline.
32. A composition comprising a
   i) a checkpoint inhibitor; and
   ii) an oncolytic Herpes Simplex Virus (HSV) comprising recombinant DNA, wherein the recombinant DNA comprises:
a) a gene comprising a 5′ untranslated region and a HSV-1, or HSV-2, VP5 gene that is operably linked to an VP5 promoter comprising a TATA element;
b) a tetracycline operator sequence positioned between 6 and 24 nucleotides 3′ to said TATA element, wherein the VP5 gene lies 3′ to said tetracycline operator sequence;
c) a gene sequence encoding tetracycline repressor operably linked to an HSV immediate-early promoter, wherein the gene sequence is located at the ICP0 locus;
d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and
e) a gene sequence encoding a functional ICP34.5 protein;
wherein said oncolytic HSV does not encode functional ICP0 and does not contain a ribozyme sequence located in said 5′ untranslated region of VP5.
33. A composition comprising
i) a checkpoint inhibitor; and
ii) an oncolytic Herpes Simplex Virus (HSV) comprising recombinant DNA, wherein the recombinant DNA does not encode functional ICP0; and encodes fusogenic activity.
34. The composition of any preceding paragraph, further comprising a pharmaceutical acceptable carrier.
35. The composition of any preceding paragraph, further comprising an agent that regulates the tet operator-containing promoter.
36. The composition of any preceding paragraph, wherein the agent is doxycycline or tetracycline
37. The composition of any preceding paragraph, wherein the checkpoint inhibitor is selected from the group consisting of: an anti-PD-1 antibody or antibody reagent, an anti-PD-L1 antibody or antibody reagent, an anti-OX40 antibody or antibody reagent, a CTLA-4 antibody or antibody reagent, a TIM-3 antibody or antibody reagent, and a TIGIT antibody or antibody reagent.

### Examples

### Introduction

Using the T-REx™ gene switch technology invented by the inventors, a novel class of tetracycline-regulatable fusogenic HSV-1 oncolytic virus, QREO5-F, was developed for cancer immunotherapy (PCT Application No. PCT/US2019/062527, which is incorporated herein by reference in its entirety). Data described herein showed that infection of multiple human cancer cell types (breast, brain, kidney, liver, lung, melanoma, ovarian, and pancreatic) with QREO5-F led to 36,000- to 5×10⁷-fold tetracycline-dependent progeny virus production, while little viral replication and virus-associated cytotoxicity were seen in infected growing as well as growth-arrested normal human fibroblasts. Specifically, QREO5F exhibited up to more than 500,000-fold more efficient viral replication in human cancer cells than in normal human fibroblasts. Using syngeneic bilateral mouse cancer models, it was demonstrated that intratumoral injection of QREO5-F is highly effective against both injected and un-injected distal CT26.WT colon carcinoma tumor in immune-competent mice, and can induce effective tumor-specific immunity that completely prevented the tumor growth following re-challenge with the same type of tumor cells. Data described herein showed that the efficacy of QREO5F in the preclinical mouse model of cancer immunotherapy can be significantly enhanced with anti-PD-L1 blockade. Specifically, it was shown herein that a combination of QREOF virotherapy and anti-PD-L1 blockade is highly effective against large inoculated and uninoculated distal CT26.WT tumors in immune competent Balb/C mice, in which 7 of 8 mice are tumor-free following combination therapy. Importantly, PD-L1 blockade alone had no therapeutic effect in these experiments.

### Materials and Methods

Mice and experimental tumors. Female BALB/c mice 6-7 weeks of age were purchased from Charles River Laboratories (Cambridge, MA). Mice were housed in metal cages at four mice per cage and maintained on a 12-h light/dark cycle. Mice were allowed to acclimatize for one week prior to experimentation. All animal experiments conducted in this study were approved by the Harvard Medical Area Standing Committee on Animals and the American Veterinary Medical Association, which is accredited by the Association for Assessment and Accreditation of Laboratory Animal Care (AAALAC) and meets National Institutes of Health standards as set forth in “The Guide for the Care and Use of Laboratory Animals.”

A syngeneic mouse colon carcinoma model was established by implantation s.c. of 5×10⁵ CT26.WT cells in a volume of 100 μl in both the left and right flanks of female BALB/c mice. Once tumors reached to ˜100 to 200 mm³, mice were randomly divided into 2 groups, and larger tumors on one side of flanks were intratumorally injected with 100 ul of DMEM containing 1 μg of doxycycline (n=8), or QREO5-F at 1×10⁷ PFU containing 1 ug doxycycline in a volume of 100 ul (n=14). The number of PFU used herein was based on the titer on the ICP0-expressing Vero cells monolayers in the presence of tetracycline. Tumors were received the same treatment on days 3 and 6 post initial inoculation. Tumor volumes were quantified every 2-3 days using a digital caliper and the formula V=(L×(W)²)/2. Data are presented as means±SEM.

Combination therapy. Bilateral CT26.WT tumors were established in female BALB/c mice as described above. Once the tumors reached to ˜90 mm³ to 180 mm³, mice were randomly divided into 4 groups, and larger tumors on one side of flanks were intratumorally injected with 100 ul of DMEM containing 1 ug of doxycycline, QREO5-F at 1×10⁷ PFU containing 1 ug doxycycline in a volume of 100 ul, or QREO5-F at 1×10⁷ PFU containing 1 ug doxycycline in a volume of 100 ul and i.p. injection of anti-PD-L1 antibody (Atezolizumab) at 200 μg/mouse. As a negative control, a group of mice (n=5) was received i.p. injection of anti-PD-L1 antibody only (200 μg/mouse). Individual groups of mice were received the same treatment on days 3 and 6 post initial inoculation. For mice receiving PD-L1 blockade, a fourth injection were carried out on day 9 post initial treatment at the same dose.

### Results

The inventors previously showed that intratumoral inoculation of QREO5-F into pre-established CT26.WT tumors lead to a markedly reduction in overall tumor growth in QREO5-F treated tumors, of particular, in the QREO5-F treated tumor with local co-delivery of 1 μg of doxycycline (PCT Application No. PCT/US2019/062527). There was an average of 11.2-fold reduction in tumor volume in QREO5-F-treated tumor in the presence of doxycycline compared to that of DMEM-treated group on day 21 post-QREO5-F virotherapy. Importantly, QREO5-F virotherapy led to a 3.2-fold reduction in growth of the contralateral tumors that received no viruses compared to that of DMEM-treated mice, suggesting that intratumoral inoculation of QREO5-F can elicit an effective anti-tumor specific immunity that can limit the growth of disseminating tumors.

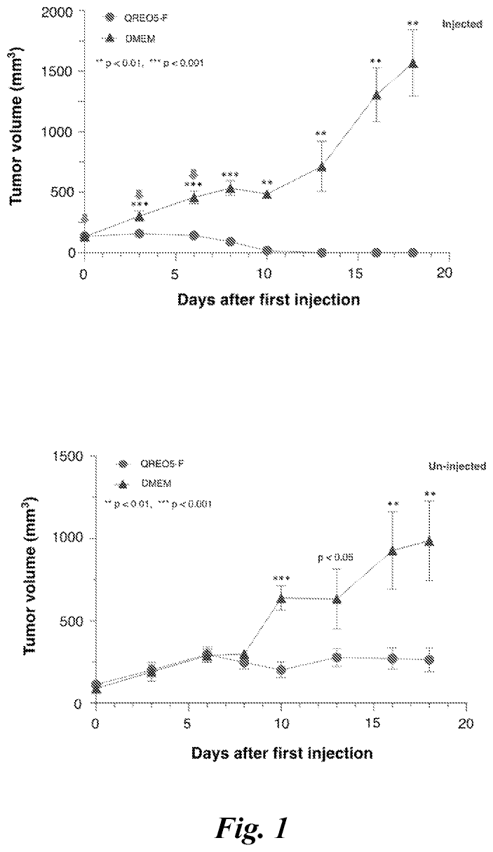

To investigate whether QREO5F possesses a similar therapeutic efficacy in the treatment of large CT26.WT tumors, bilateral CT26.WT tumors were established and once tumor volume reached to about 100 to 200 mm³, the larger tumor on one side of flanks was injected with either DMEM or QREO5F in the presence of 1 μg of doxycycline. Results presented in, e.g., FIG. 1, showed that all QREO5F-injected tumors completely regressed by day 13 post-treatment, while the overall growth of distal non-treated contralateral tumors in QREO5F-treated mice was reduced 3.8-fold compared with contralateral non-injected tumors in DMEM-treated mice on day 18 post-treatment (p=0.003). One of eight QREO5F-treated mice became tumor free on both flanks on day 10 post-virotherapy.

Combination of QREO5F and PD-L1 blockade significantly enhances the efficacy of QREO5F in the treatment of both injected and un-injected distal tumors in mice bearing large CT26.WT tumors.

Atezolizumab is an anti-PD-L1 monoclonal antibody currently approved for the treatment of various forms of cancers. Atezolizumab has high affinity for both human and mouse PD-L1 (Irving B, Chiu H et al., 2012-In Office USP. Ed (USA:Genentech, Inc.); Powles T Eder J P et al., Nature, 2014).

To investigate the effects of QREO5F and atezolizumab combination therapy in preclinical models of cancer immunotherapy, large bilateral CT26.WT tumors were established in normal BALB/c mice. Once the tumors reached to ˜ 90 mm³ to 180 mm³, mice were randomly divided into 4 groups, and the larger tumor on one side of flanks were intratumorally injected with DMEM containing 1 ug of doxycycline, QREO5-F containing 1 ug doxycycline only, or QREO5-F containing 1 ug doxycycline in combination with i.p. injection of atezolizumab. As a control, a group of mice was treated with a single atezolizumab therapy.

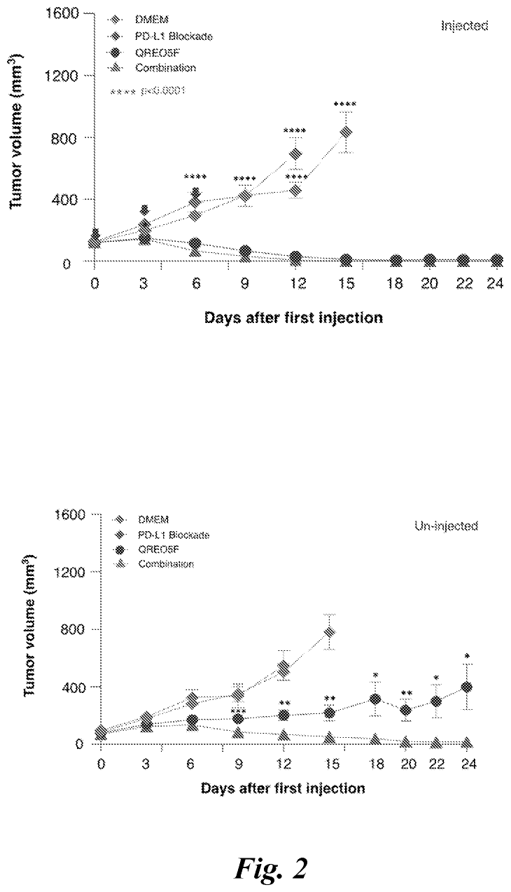

The results presented in FIG. 2 showed that single atezolizumab therapy had no effect on the tumor growth as compared with DMEM treated mice and three of five mice has to be euthanized on day 12 post-treatment due to the large tumor volume. Consistent with study presented in FIG. 1, intratumoral injection of QREO5F in mice treated with QREO5F alone led to 5 of 7 injected tumors completely regressed by day 15 post-treatment, and the remaining 2 tumors almost completely regressed at the time of euthanasia on day 24 and day 28 post treatment, respectively. The overall growth of distal non-treated contralateral tumors in these mice was reduced 3.6-fold compared with contralateral non-injected tumors in DMEM-treated mice on day 15 post-treatment (p<0.001). One of seven QREO5F-treated mice became tumor free on both flanks on day 26 post-virotherapy. Compared with mice treated with single QREO5F therapy, all injected tumors in mice received combination therapy of QREO5F and atezolizumab were completely regressed by day 18 post-treatment, and the rate in overall tumor regression in distal tumors in mice treated with combination therapy was significantly faster than mice treated with single virotherapy starting on day 9 post treatment (p<0.005, p<0.01, or P<0.05). Seven of 8 distal non-treated contralateral tumors were completely regressed by day 24 post-treatment in mice treated with combination therapy, while the remaining one tumor showed little tumor growth during the 24-day period of observation. Collectively, the combination of QREO5F virotherapy and anti-PD-L1 blockade is highly effective against large inoculated and uninoculated distal CT26.WT tumors, in which 7 of 8 mice are tumor-free following the combination therapy.

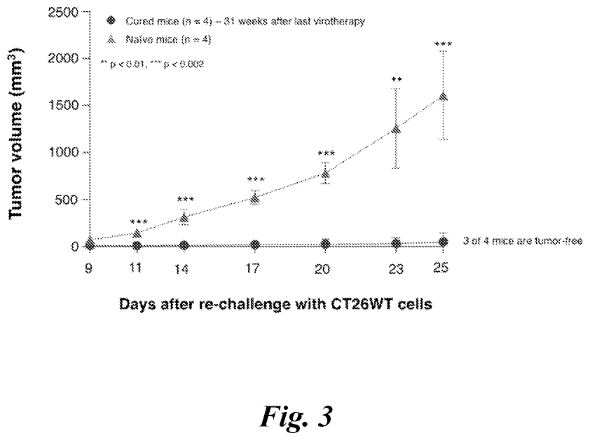

To evaluate the induction of tumor-specific memory response following QREO5F and anti-PD-L1 combination therapy, we re-challenged four combination therapy cured mice with CT26WT cells at 31 weeks after last QREO5F therapy. As a positive control, 5 naïve female BALB/c mice were also s.c. injection of CT26WT cells. Three of four cured tumor-free mice had no detectable tumor growth by day 25 post-CT26WT cells challenge, while there was a small tumor developed in the remaining one mouse with size much smaller than tumors seen in five normal naïve mice (FIG. 3).

These results indicate that intratumoral injection of QREO5F in combination with anti-PD-L1 blockade is capable of eliciting long-lasting systemic tumor-specific memory immune response that can effectively prevent tumor recurrence and tumor growth following re-challenge with the same type of tumor cells.

1. A method for treating cancer, the method comprising administering a subject in need thereof,
i) a checkpoint inhibitor; and
ii) an oncolytic Herpes Simplex Virus (HSV) comprising recombinant DNA, wherein the recombinant DNA comprises:
a) a gene comprising a 5′ untranslated region and a HSV-1, or HSV-2, VP5 gene that is operably linked to an VP5 promoter comprising a TATA element;
b) a tetracycline operator sequence positioned between 6 and 24 nucleotides 3′ to said TATA element, wherein the VP5 gene lies 3′ to said tetracycline operator sequence;
c) a gene sequence encoding tetracycline repressor operably linked to an HSV immediate-early promoter, wherein the gene sequence is located at the ICP0 locus;
d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and
e) a gene sequence encoding a functional ICP34.5 protein;
wherein said oncolytic HSV does not encode functional ICP0 and does not contain a ribozyme sequence located in said 5′ untranslated region of VP5.

2. The method of claim 1, wherein the cancer is a solid tumor.

3. (canceled)

4. (canceled)

5. The method of claim 1, wherein the subject is diagnosed or has been diagnosed as having cancer is selected from the list consisting of: a carcinoma, a melanoma, a sarcoma, a germ cell tumor, and a blastoma.

6. The method of claim 1, wherein the subject is diagnosed or has been diagnosed as having a cancer selected from the group consisting of: non-small-cell lung cancer, breast cancer, brain cancer, colon cancer, prostate cancer, liver cancer, lung cancer, ovarian cancer, skin cancer, head and neck cancer, kidney cancer, and pancreatic cancer.

7. The method of claim 1, wherein the cancer is metastatic.

8. The method of claim 1, wherein the variant gene is a gK variant gene that encodes an amino acid substitution selected from the group consisting of: an Ala to Thr amino acid substitution corresponding to amino acid 40 of SEQ ID NO: 2; an Ala to “x” amino acid substitution corresponding to amino acid 40 of SEQ ID NO: 2, wherein “x” is any amino acid; an Asp to Asn amino acid substitution corresponding to amino acid 99 of SEQ ID NO: 2; a Leu to Pro amino acid substitution corresponding to amino acid 304 of SEQ ID NO: 2; and an Arg to Leu amino acid substitution corresponding to amino acid 310 of SEQ ID NO: 2.

9. The method of claim 1, wherein the tetracycline operator sequence comprises two Op2 repressor binding sites.

10. The method of claim 1, wherein the VP5 promoter is an HSV-1 or HSV-2 VP5 promoter.

11. The method of claim 1, wherein the immediate-early promoter is an HSV-1 or HSV-2 immediate-early promoter.

12. The method of claim 11, wherein the HSV immediate-early promoter is selected from the group consisting of: ICP0 promoter, ICP27 promoter and ICP4 promoter.

13. The method of claim 1, wherein the recombinant DNA is part of the HSV-1 genome or the HSV-2 genome.

14. (canceled)

15. The method of claim 1, further comprising administering an agent that regulates the tet operator-containing promoter.

16.-18. (canceled)

19. The method of claim 1, wherein at least one of:
the checkpoint inhibitor and the oncolytic virus are administered directly to the tumor,
the checkpoint inhibitor is administered systemically and the oncolytic virus are administered directly to the tumor,
the checkpoint inhibitor and the oncolytic virus are administered in the same composition,
the checkpoint inhibitor and the oncolytic virus are administered in different compositions,
the checkpoint inhibitor and the oncolytic virus are administered at substantially the same time, and
the checkpoint inhibitor and the oncolytic virus are administered at different times.

20.-24. (canceled)

25. The method of claim 1, wherein the checkpoint inhibitor is selected from the group consisting of: an anti-PD-1 antibody or antibody reagent, an anti-PD-L1 antibody or antibody reagent, an anti-OX40 antibody or antibody reagent, a CTLA-4 antibody or antibody reagent, a TIM-3 antibody or antibody reagent, and a TIGIT antibody or antibody reagent.

26. (canceled)

27. A method for treating cancer, the method comprising administering a subject in need thereof,
i) a checkpoint inhibitor; and
ii) an oncolytic Herpes Simplex Virus (HSV) comprising recombinant DNA, wherein the recombinant DNA does not encode functional ICP0; and encodes fusogenic activity.

28.-31. (canceled)

32. A composition comprising a
i) a checkpoint inhibitor; and
ii) an oncolytic Herpes Simplex Virus (HSV) comprising recombinant DNA, wherein the recombinant DNA comprises:
a) a gene comprising a 5′ untranslated region and a HSV-1, or HSV-2, VP5 gene that is operably linked to an VP5 promoter comprising a TATA element;
b) a tetracycline operator sequence positioned between 6 and 24 nucleotides 3′ to said TATA element, wherein the VP5 gene lies 3′ to said tetracycline operator sequence;
c) a gene sequence encoding tetracycline repressor operably linked to an HSV immediate-early promoter, wherein the gene sequence is located at the ICP0 locus;
d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; and
e) a gene sequence encoding a functional ICP34.5 protein;
wherein said oncolytic HSV does not encode functional ICP0 and does not contain a ribozyme sequence located in said 5′ untranslated region of VP5.

33. A composition comprising
i) a checkpoint inhibitor; and
ii) an oncolytic Herpes Simplex Virus (HSV) comprising recombinant DNA, wherein the recombinant DNA does not encode functional ICP0; and encodes fusogenic activity.

34. (canceled)

35. The composition of claim 32, further comprising an agent that regulates the tet operator-containing promoter.

36. The composition of claim 35, wherein the agent is doxycycline or tetracycline.

37. The composition of claim 32, wherein the checkpoint inhibitor is selected from the group consisting of: an anti-PD-1 antibody or antibody reagent, an anti-PD-L1 antibody or antibody reagent, an anti-OX40 antibody or antibody reagent, a CTLA-4 antibody or antibody reagent, a TIM-3 antibody or antibody reagent, and a TIGIT antibody or antibody reagent.
